(19) United States
(12) Patent Application Publication
(10) Pub. No.: US 2025/0115953 A1
(43) Pub. Date: Apr. 10, 2025

(54) Methylcytosine-Selective Deaminases and Uses Thereof

(71) Applicant: New England Biolabs, Inc., Ipswich, MA (US)

(72) Inventors: Laurence Ettwiller, Beverly, MA (US); Peter R. Weigele, Cambridge, MA (US); Yan-Jiun Lee, Salem, MA (US); Weiwei Yang, Peabody, MA (US); Amanda N. Deliberto, Quincy, MA (US); Rebekah M. B. Silva, Georgetown, MA (US); Sean R. Johnson, Beverly, MA (US); Colleen E. Yancey, Melrose, MA (US)

(73) Assignee: New England Biolabs, Inc., Ipswich, MA (US)

(21) Appl. No.: 18/908,725

(22) Filed: Oct. 7, 2024

Related U.S. Application Data
(60) Provisional application No. 63/588,121, filed on Oct. 5, 2023.

(51) Int. Cl. C12Q 1/6869; C12N 9/80; C12N 15/11; C12Q 1/34; C12Q 1/6806; C12Q 1/6855; C12Q 1/686

(52) U.S. Cl. CPC C12Q 1/6869; C12N 9/80; C12N 15/111; C12Q 1/34; C12Q 1/6806; C12Q 1/6855; C12Q 1/686; C07K 2319/80; C12Q 2600/154; C12Y 305/04001; G01N 2333/98

(57) ABSTRACT

Provided are methylcytosine-selective deaminases, compositions, kits, and methods employing them, including a sequencing method involving (a) contacting a DNA substrate comprising cytosine (C) and at least one methylcytosine nucleobase selected from 5-methylcytosine (5mC) and 5-hydroxymethylcytosine (5hmC) or comprising C and both 5mC and 5hmC, with a methylcytosine-selective deaminase to produce a deamination product, wherein the methylcytosine-selective deaminase (i) is capable of deaminating 5mC to thymidine (T) and/or 5hmC to hydroxymethyluridine (hmU) and (ii) preferentially deaminates the at least one methylcytosine nucleobase relative to cytosine (C); and (b) sequencing the deamination product, or amplifying the deamination product to produce an amplification product, and sequencing the amplification product, in each case, to produce sequence reads, wherein positions of Cs and position of the at least one methylcytosine nucleobase in the DNA substrate is determined based on the sequence reads.

### SEQUENCE LISTING STATEMENT

This disclosure includes a Sequence Listing submitted electronically in .xml format under the file name “NEB-482.xml” created on Oct. 7, 2024, and having a size of 61.7 KB. This Sequence Listing is incorporated herein in its entirety by this reference.

### BACKGROUND

Epigenetic modification of genomic DNA has a fundamental role in development and gene regulation of prokaryotes and eukaryotes. Methylation of the nucleobase cytosine (C) is the most studied type of epigenetic modification and is increasingly used as a diagnostic marker for human disease. In mammals, cytosine modification in regions of the mammalian genome that are rich in C and the nucleobase guanine (G) has a significant role in epigenetic regulation. The most important modification is conversion of cytosine to 5-methylcytosine (5mC), although oxidized forms of 5mC (such as 5-hydroxymethylcytosine) are recognized as important components of epigenomic shaping. Many DNA sequencing approaches exist for distinguishing various forms of methylcytosine (mC) from C. These approaches rely on conversion of C and/or mC to another nucleobase by deamination followed by bioinformatics analysis to identify which nucleobases were converted.

One of the earliest established approaches involves deaminating DNA samples using bisulfite, which converts C to the nucleobase uracil (U), whereas mC is not converted. Because DNA is double-stranded and C-G is the normal pairing for C, the conversion of C to U creates a mismatch in the double-stranded DNA sequence. This mismatch of converted C to U (which will be read as a T by a sequencing instrument) is used to distinguish C from 5mC (which ultimately will be read as a C by a sequencing instrument). To overcome technical disadvantages of using bisulfite, such as chemical degradation of DNA samples, enzymatic deamination was developed. This approach employs deaminase enzymes to gently convert Cs to Us. To protect mC from being converted, DNA samples are pretreated with oxidase enzymes such as TET and/or glucosidases such as BGT in various combinations to generate products that are resistant to enzymatic deamination. As with bisulfite sequencing, Cs are read as Ts by the sequencing instrument (as are unprotected mCs) and protected mCs are read as Cs. Such pretreatments add steps to the standard DNA sequencing workflow, which in turn requires higher input amounts of DNA, consumes processing time, and inherently has more failure points.

An epigenetic sequencing workflow that employs a methylcytosine-selective deaminase to selectively convert mCs, such as 5mC, to T and leave Cs substantially unaltered would be a more direct and efficient approach. Further, a methylation-selective deaminase would be useful in gene editing applications in which mC to T conversion is desirable, as well as in a variety of other applications.

### SUMMARY

Provided herein is a sequencing method, involving (a) contacting a DNA substrate comprising cytosine (C) and at least one methylcytosine nucleobase selected from 5-methylcytosine (5mC) and 5-hydroxymethylcytosine (5hmC) or comprising C and both 5mC and 5hmC, with a methylcytosine-selective deaminase to produce a deamination product, wherein the methylcytosine-selective deaminase (i) is capable of deaminating 5mC to thymidine (T) and/or 5hmC to hydroxymethyluridine (hmU) and (ii) preferentially deaminates the at least one methylcytosine nucleobase relative to cytosine (C); and (b) sequencing the deamination product, or amplifying the deamination product to produce an amplification product, and sequencing the amplification product, in each case, to produce sequence reads, wherein positions of Cs and position of the at least one methylcytosine nucleobase in the DNA substrate is determined based on the sequence reads.

In an embodiment, DNA substrate does not comprise DNA that has been pretreated to protect any nucleobase from deamination. In an embodiment, DNA substrate does not comprise DNA that has been pretreated to protect 5mC from deamination. In an embodiment, DNA substrate does not comprise DNA that has been pretreated with a TET methylcytosine dioxygenase.

In an embodiment, DNA substrate does not comprise DNA that has been pretreated to protect C from deamination. In an embodiment, the methylcytosine-selective deaminase preferentially deaminates 5mC relative to 5hmC and C, and the position of at least one 5mC in the DNA substrate is determined. In an embodiment, the DNA substrate comprises DNA that has been pre-treated to protect one or more methylcytosine nucleobases from deamination. Such pretreatment may comprise incubation with an enzyme. In an embodiment, the enzyme protects 5hmC from deamination. In this embodiment, at least one 5mC, at least one 5hmC, and C may be determined. In an embodiment, enzyme is selected from beta-glucosyltransferase (BGT), carbamoyl transferase, glycotransferase selective for 5hmC, and a combination thereof. In a specific embodiment, the enzyme is BGT and the methylcytosine-selective deaminase does not deaminate glucosylated hmC. In an embodiment, the enzyme protects C from unwanted background deamination by the methylcytosine-selective deaminase. In an embodiment, such an enzyme is selected from 4mC methylase and N4 methyltransferase. In an embodiment, the enzyme protects 5mC from deamination, and in specific embodiment, the enzyme is TET methylcytosine dioxygenase and the methylcytosine-selective deaminase does not deaminate 5-formylC (5fC) or 5-carboxyC (5caC). In an embodiment, the pretreatment comprises incubation with a chemical reagent, and in a specific embodiment the chemical reagent is pyridine borate.

In an embodiment, the methylcytosine-selective deaminase of (a) is a mixture of two or more methylcytosine-selective deaminases.

In an embodiment, the methylcytosine-selective deaminase of (a) does not deaminate C above background level. In an embodiment, the methylcytosine-selective deaminase is selective for a methylcytosine nucleobase present within a defined sequence context, wherein the methylcytosine-selective deaminase does not deaminate the methylcytosine nucleobase lacking the defined sequence context above background level.

In an embodiment, the methylcytosine-selective deaminase comprises more than one methylcytosine-selective deaminase, each selective for 5mC and/or 5hmC present within different sequence contexts, wherein the methylcytosine selective deaminases do not deaminate the 5mC and/or 5hmC lacking the sequence context above background level.

In an embodiment, prior to (b) the DNA substrate is contacted with one or more deaminating agents having a different specificity from the methylcytosine-selective deaminase of (a). In an embodiment, the deaminating agent is selected from an enzyme, a chemical reagent, and both.

In an embodiment, the deamination product is not amplified. In this embodiment, the deamination product can be directly added to the sequencing machine or alternatively can be further processed prior to adding to the sequencing machine. In another embodiment, the deamination product is amplified to produce an amplification product. In an embodiment, the amplification involves using polymerase chain reaction and in an embodiment, the polymerase chain reaction is performed using a polymerase having a uracil-binding pocketQ5U polymerase. In a specific embodiment, the polymerase having a uracil-binding pocketQ5U polymerase is Q5.

In an embodiment, before (b), the deamination product is contacted with a substance that generates a single nucleotide gap at the location of a uracil (U). In an embodiment the substance is selected from USER enzyme.

In another embodiment before (b), the deamination product is contacted with a substance that generates an abasic site at the location of a U and/or an 5hmU. In an embodiment the substance is selected from uracil DNA glycosylase, single-strand-selective monofunctional uracil DNA glycosylase 1, thymine-DNA glycosylase and methyl-CpG binding domain protein 4.

In an embodiment, DNA substrate further comprises an adaptor. The adaptor can further comprises a sequencing primer binding site. In an embodiment, the adaptor can also comprise a barcode and/or unique molecular identifier (UMI).

Any DNA substrate suspected of containing C and 5mC and/or 5hmC can be used in the described sequencing method. In various embodiments, the DNA substrate comprises DNA selected from genomic DNA, ancient DNA, forensic DNA, environmental DNA, human DNA, cell free DNA, synthetic DNA, plant DNA, and tumor DNA. In an embodiment, the DNA substrate further comprises RNA. The DNA substrate can contain methylcytosine nucleobases (e.g., 5mC and/or 5hmC) at low allelic fractions.

The sequencing methods involve using a methylcytosine-selective deaminase, which, in an embodiment is a viral deaminase or non-natural modification thereof. In an embodiment, the viral deaminase is a bacteriophage-encoded deaminase or modification thereof.

In an embodiment, the methylcytosine-selective deaminase comprises a B5 family member signature sequence. In various embodiments,
the methylcytosine-selective deaminase comprises an amino acid sequence that is at least 90% identical to any of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55. In specific embodiments, the methylcytosine-selective deaminase comprises an amino acid sequence selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54, and 55., such as a methylcytosine-selective deaminase comprises an amino acid selected from SEQ ID NOS:1, 5 and 14.

In an embodiment for generating a DNA substrate comprising epigenetic and genomic sequence, the method can involve comprising preparing the DNA substrate prior to (a) in a method comprising: (a) ligating a hairpin adaptor to a double-stranded genomic DNA sample to produce a ligation product; (b) enzymatically generating a free 3′ end in a double-stranded region of the hairpin adaptor in the ligation product; (c) extending the free 3′ end in a reaction mix that comprises a strand-displacing or nick-translating polymerase, dGTP, dATP, dTTP and modified dCTP, wherein the modified dCTP is deamination resistant to produce and extended product; to thereby produce a single-stranded DNA substrate.

Provided herein is a method for selective deamination of a DNA substrate. The resultant deamination product can be used in a variety of applications, including detection of target C, 5mC and/or 5hmC, detection of bulk methylation status, and variations. In an embodiment, the method involves contacting a DNA substrate comprising cytosine (C) and at least one methylcytosine nucleobase selected from 5-methylcytosine and 5-hydroxymethylcytosine, with a methylcytosine-selective deaminase, to produce a deamination product, wherein the methylcytosine-selective deaminase (i) is capable of deaminating 5mC to thymidine (T) and/or 5hmC to hydroxymethyluridine (hmU), (ii) preferentially deaminates 5mC and/or 5hmC relative to cytosine (C), and (iii) and is characterized by having at least one of the features selected from: (a) the methylcytosine-selective deaminase comprises a B5 family signature sequence; and (b) the methylcytosine-selective deaminase is encoded by a viral gene located in a viral genome in proximity to a thymidylate synthase gene, and (c) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55.

Also provided is a method for nucleobase editing, using the methylcytosine-selective deaminase described herein. The method involves contacting a fusion protein comprising a methylation-selective deaminase capable of deaminating 5mC to thymidine (T) fused to a DNA binding domain, with a target sequence comprising cytosine (C) and one or more 5-methylcytosine (5mC), to produce an edited target sequence comprising at least one T generated by deaminating 5mC, wherein the methylation-selective deaminase preferentially deaminates 5mC relative to C, and is characterized by having at least one of the features selected from: (i) the methylcytosine-selective deaminase comprises a B5 family deaminase signature sequence and (ii) the methylcytosine-selective deaminase is encoded by a viral gene located in a viral genome in proximity to a thymidylate synthase gene, and (iii) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55. In an embodiment, methylcytosine-selective deaminase is selective for 5mC present within a sequence context, wherein the methylcytosine-selective deaminase does not deaminate 5mC lacking the sequence context above background level. In an embodiment, the DNA binding domain is selected from a Cas9 domain, a Cas12 domain, a transcription activator-like effector nuclease (TALEN domain), a zinc finger (ZF) domain, a transcription activator-like effector (TALE) domain, and a methyl binding domain (MBD) domain.

Also provided herein is a kit for methylation sequencing. The kit includes at least (a) a methylcytosine-selective deaminase that (i) is capable of deaminating 5-methyl cytosine (5mC) to thymidine (T) and/or 5-hydroxylmethyl cytosine (5hmC) to hydroxymethyluridine (hmU) and (ii) preferentially deaminates 5mC and/or 5hmC relative to cytosine (C); and
(b) one or more items selected from a primer; adaptor, polymerase; reverse transcriptase; ligase; protecting agent for selected nucleobase and/or modified nucleobase; another deaminase and/or deaminating treatment; reference DNA, methylated reference DNA; reference sequence; reaction buffer.

In an embodiment, the methylcytosine-selective deaminase is characterized by having at least one of the features selected from (i) the methylcytosine-selective deaminase comprises a B5 family member signature sequence; and (ii) the methylcytosine-selective deaminase is encoded by a viral gene located in a viral genome in proximity to a thymidylate synthase gene, and (iii) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 53, 54 and 55.

Also provided is a methylcytosine-selective deaminase. The deaminase comprises a polypeptide that is capable of deaminating 5-methyl cytosine (5mC) to thymidine (T) and/or 5-hydroxylmethyl cytosine (5hmC) to hydroxymethyluridine (hmU); and preferentially deaminates 5mC and/or 5hmC relative to cytosine (C); wherein the polypeptide is selected from: a non-natural fusion protein comprising a B5 deaminase family signature sequence; a non-natural fusion protein comprising a methylcytosine-selective deaminase encoded by a viral gene located in a viral genome in proximity to a thymidylate synthase gene; a non-natural fusion protein comprising an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 53, 54 and 55; and a polypeptide comprising an amino acid sequence selected from SEQ ID NOS: 37, 38, 39, and 40.

In an embodiment, the polypeptide is a non-natural fusion protein comprising a B5 family member signature sequence, and the methylcytosine-selective deaminase portion of the fusion protein comprises is encoded by a viral gene.

Further provided is a composition comprising such a methylcytosine deaminase together with a component selected from a solid support, a buffer, and a reaction mixture.

Such a methylcytosine deaminase can also be provided in a kit for deaminating a DNA substrate, which also comprises one or more items selected from a reaction buffer, primer; adaptor, polymerase; reverse transcriptase; ligase; protecting agent for selected nucleobase and/or modified nucleobase; another deaminase and/or deaminating treatment; reference DNA, methylated reference DNA; reference sequence; reaction buffer.

### BRIEF DESCRIPTION OF DRAWINGS

FIG. 1A-1B shows chromatograms of HPLC separated nucleosides obtained from E. coli strains expressing methylcytosine-selective deaminases C4, F4, B5, D5, E8, E10, and H10 together with a functional hydroxymethyl-dC (5hmdC) biosynthetic pathway. FIG. 1A shows labeled peaks indicating levels of dC, dG, dT, dA, 5-hmdC, and 5hmdU. Presence of 5-hmdU indicates presence of 5-hmdC deaminating activity. FIG. 1B shows labeled peaks indicating levels of dC, 5mdC, dG, dT, and dA. Presence of 5mdC indicates presence of 5mdC deaminating activity.

FIG. 2 shows activities of purified B5 deaminase, and control APOBEC, on bacteriophage T4gt genomic DNA polymers in which all Cs are substituted with 5hmC.

FIG. 3 shows activity of B5 deaminase and control APOBEC on a mixture of DNA polymer substrates containing 5mC or 5hmC.

FIG. 4A-4B shows activity of B5 deaminase and control ABOBEC on oligonucleotides containing C and 5mC (FIG. 4A) or containing C and 5hmC (FIG. 4B).

FIG. 5A-5E shows activity of B5 deaminase on nucleotide triphosphates of C, 5mC, and 5hmC.

FIG. 6 shows activity of B5 deaminase on ribonucleotides.

FIG. 7A-7G shows a schematic representation of an assay for detecting whether 5mC contained in an oligonucleotide is selectively deaminated (FIG. 7A) and capillary electrophoresis mobility shifts of oligonucleotides cleaved by EcoRI after converting 5mCs to Ts using four exemplary methylcytosine-selective deaminases described herein, including B5 deaminase (FIG. 7B); E10 deaminase (FIGS. 7C and 7D); PCBV1 deaminase (FIG. 7E); and C73 deaminase (FIG. 7F)) and B5 deaminase (FIG. 7G).

FIG. 8A-8B shows results of sequencing DNA purified from E. coli K12 (dcm+) after deamination using B5 deaminase. FIG. 8A shows C to T transition as darker color blocks and indicates percentage of C to T conversions for two sequence contexts (CCAGG and CCTGG) when the deamination reaction was carried out at 42 (upper panel), 37 (middle panel) and 30 (bottom panel) degrees C.

FIG. 8B shows overall C to T transition rates across the genome, where the X axis represents the possible contexts (X_BASE_XXX, with X=ATCG and BASE=C). As shown, most of the C to T transition are observed in the CCAGG (1) or CCTGG (2) context.

FIG. 9 shows for each cytosine position in the genome of Neisseria meningitidis (left panel), Lambda phage (center left panel), T4gt phage (center right panel), and XP12 phage (right panel), the total number of reads mapping at a position (x-axis) compared to the log 10 of the C to T frequency in the reads (the frequency is calculated as the number of reads showing a C to T variants of high base quality score (Q>30) divided by the total number of read mapping to the position). The cutoff of 0.25% of C to T is calculated to maximize and minimize the number of positions above the cutoff for XP12 and Lambda respectively. Any site below this cutoff is considered unmethylated whereas above the cutoff is considered methylated. The percentage of C sites considered methylated is 2.78%, 0.11%, 99.3% and 99.73% for Neisseria meningitidis, Lambda phage, T4gt phage and XP12 phage respectively.

FIG. 10A-10C shows allelic frequency of C to T transitions in Neisseria meningitidis relative to read coverage at TCTGG and TTCCGG sites (with C being the interrogated base). FIG. 10A shows that for the C in TCTGG context in the Neisseria meningitidis genome, the total number of reads mapping at this position (x-axis) is compared to the log 10 of the C to T frequency in the reads (the frequency is calculated as the number of reads showing a C to T variants of high base quality score (Q>30) divided by the total number of reads mapping to the position). The dashed line corresponds to 0.25% of C to T. At this cutoff, 100% and 94.65% of C in TCTGG and TTCCGG contexts are methylated respectively. FIG. 10B shows an IGV plot representation of the reads (grey) mapping to a specific TCTGG site in the Neisseria meningitidis genome. Black lines represent reads with C to T variants. FIG. 10C Shows an IGV plot representation of the reads (grey) mapping to a specific TTCCGG site in the Neisseria meningitidis genome. Black lines represent reads with C to T variants.

FIG. 11A-11C shows for every C in the T4 (5hmC, left panels), XP12 (5mC, middle panels) and Lambda (C, right panels) genomes, the read coverage at that position (x-axis) is a function of the fraction of reads showing C to T transition (y-axis) for the mixed sample treated with BGT (upper panels) or no treatment (lower panels). In this experiment, a C to T fraction >0.6% is considered mC.

FIG. 12 shows the amount of 5mC (x-axis) relative to deamination of 5mC by B5 deaminase as reflected by the C to T ratio (Y axis) in substrate oligonucleotide mixtures containing 0%, 0.5%, 1%, 2%, 5%, 10%, 20%, 40%, 80% or 100% 5mC at a single position.

FIG. 13 shows capillary electrophoresis mobility shifts of oligonucleotides cleaved by EcoRI after incubation with B5 deaminase immobilized on Ni-NTA magnetic beads. Peaks labelled −9999 correspond to fully cleaved oligonucleotides and peaks labelled 20.6 (retention time) correspond to uncleaved oligonucleotides.

FIG. 14A-14B shows time courses of B5 deamination of 5mC in a single stranded DNA substrate containing both 5mC and 5hmC using different enzyme to substrate ratios; FIG. 14A shows conversion of 5mC to dT; FIG. 14B shows conversion of C to dU.

FIG. 15 shows a schematic representation of an embodiment of a library preparation method employing a methylcytosine-selective deaminase.

FIG. 16A-16E shows deamination rates of B5 deaminase under various conditions including denaturation conditions (FIG. 16A), addition of zinc (FIG. 16B), buffer conditions (FIG. 16C), temperature (FIG. 16D), and enzyme substrate ratios (FIG. 16E).

FIG. 17A-17B shows B5 deamination rates of genomic DNA from XP12, T4gt and Lambda phage mixture (FIG. 17A) when using Q5 polymerase, Q5U polymerase or Q5 plus USER. Deamination was even across the sequencing reads for mC (FIG. 17B, top), 5hmC (FIG. 17, middle), and C (FIG. 17B, bottom).

FIG. 18 shows B5 selective deamination of 5mC using Q5, Q5 plus USER and Q5U polymerase conditions with various substrates: DHB4 genomic DNA, XP12 genomic DNA) and T4gt genomic DNA.

FIG. 19A-19B shows fragment size distribution of the converted library using free circulating DNA (FIG. 19A) and the average methylation levels function of the insert size (FIG. 19B), where the peak methylation level can be found for fragments of about 187 bp long.

FIG. 20 shows a molecular model of an embodiment of a methylcytosine-selective deaminase, which highlights residues in a methylcytosine-selective deaminase signature sequence S-S-Y-G-N. Amino acid numbering is with respect to SEQ ID NO:1 with a methionine added to the N-terminal.

### DETAILED DESCRIPTION

The present disclosure provides methods for using methylcytosine-selective deaminases (e.g., methylcytosine-selective deaminases that react on methylated cytosine (mC), such as 5-methylcytosine (5mC) and 5-hydroxymethylcytosine (5hmC), relative to cytosine (C) for a variety of applications, such as epigenetic sequencing, detecting methylcytosine using molecular assays, and genome editing. Also provided are methylcytosine-selective deaminases, and compositions and kits containing them.

The inventors have developed, among other aspects, a new epigenetic sequencing technique having surprising simplicity due to their recognition that methylcytosine-selective deaminase can significantly reduce assay complexity relative to existing sequencing workflows. Although a methylcytosine-selective mutant of activation induced deaminase (AID) was previously reported in 2017, the potential usefulness of such an enzyme that deaminates methylcytosine preferentially over cytosine in DNA remained unrecognized until now. While the sequencing methods described herein do not appear to have been previously been described using any enzyme, the methods can be carried out using any methylcytosine-selective deaminase having properties as described herein.

Further, the inventors have recognized the unexpected ability of certain viral mononucleotide deaminases (described herein below) to deaminate DNA polymers, and the application of these enzymes in various methods disclosed herein.

Thus, also provided are methylcytosine-specific deaminases, and unnatural variants thereof, useful for a variety of methods employing the capability of these deaminases to convert 5mC to T and/or 5hmc to 5-hydroxyuracil (5hmU), in nucleic acid polymers.

As background, a previously discovered methyl cytidine deaminase from phage XP12 has been shown to deaminate 5mC at the mononucleotide level (Wang R Y, Ehrlich M. 5-methyl-dCTP deaminase induced by bacteriophage XP12. J Virol. 1982 April; 42(1): 42-8. doi:10.1128/JVI.42.1.42-48.1982. PMID: 7086962; PMCID: PMC256042) and was not shown to deaminate 5mC in nucleic acid polymers. It was unexpected that a member of this class of enzymes would deaminate 5mC in a nucleic acid polymer, for example, because the XP12 genome contains 5mC instead of C in its genome, and deamination of 5mC would therefore disrupt its genome.

Epigenetic sequencing methods in which Cs are converted to Us result in products that are not amplified by most polymerases, necessitating the use of dU tolerant polymerases for amplification. In contrast, for the methods described herein, the 5mC to T deamination product can be amplified using a variety of polymerases that need not be dU tolerant.

The methylcytosine-selective deaminases described herein can be used in a variety of methods, particularly methods for identifying the position and/or identity of one or more modified cytosines; determining the methylation status of a cytosine; converting a methylcytosine to another nucleobase (e.g., a 5mC to T conversion or 5hmC to 5hmU conversion). Examples include obtaining sequence for genetic and epigenetic bases using a variety of published sequencing workflows and/or commercially available sequencing kits, by adding a deamination step employing a methylcytosine-selective deaminase, including long read sequencing; identifying low allelic fractions of methylation (e.g., detecting mC is a sample containing many cells or nucleic acid molecules, where a small fraction is expected to contain target nucleic acids, such as samples for detecting residual cancer cells in blood or tissue, samples of cell free DNA, amniotic fluid); assays in which primers complementary to the deaminated version of the nucleic acid are used to detect mC (e.g., PCR and LAMP); mutagenesis in vitro or in vivo to convert 5mC to T, e.g., to insert or remove a start or stop codon; partial deamination reactions to obtain a pool of unique identifiers from a DNA molecule containing multiple 5mC sites; base editing (e.g., using fusion proteins of the methylcytosine-selective deaminase to generate site-specific base changes) and other genome engineering approaches.

Deaminases such as APOBEC3 (Ito F., Fu Y., Kao S. A., Yang H., Chen X. S. Family-wide comparative analysis of cytidine and methylcytidine deamination by eleven human APOBEC proteins. Journal of Molecular Biology. 2017; 429:1787-1799; Schutsky E K, DeNizio J E, Hu P, Liu M Y, Nabel C S, Fabyanic E B, et al. Nondestructive, base-resolution sequencing of 5-hydroxymethylcytosine using a DNA deaminase. Nat Biotechnol. 2018) have been previously used for deaminating C and modified C for epigenetic sequencing workflows. Because APOBEC enzymes deaminate both mCs and Cs, sequencing workflows using these enzymes require protecting mCs from APOBEC activity and allowing APOBEC to convert Cs to Us.

In contrast, use of the methylcytosine-selective deaminases described herein allows selective deamination of mCs, e.g., conversion of 5mC to T, without fully deaminating Cs, which has not been described previously. Unwanted deamination of Cs by a methylcytosine-selective deaminase can be addressed, if desired, by cleaving at Us in the methylcytosine-selective deamination product (for example, using USER enzyme (Uracil-Specific Excision Reagent, containing uracil DNA glycosylase (UDG) and endonuclease VIII)) (New England Biolabs, Inc.) or UDG). Another approach is to protect Cs using. In the context of sequencing, this reduces background error rate. Lower background results in higher sensitivity, which is useful, for example, when sequencing samples with low allelic methylation levels and cell free DNA samples, such as for disease detection. Example 32 describes use of an embodiment of the sequencing methods described herein on cell free DNA substrates having low allelic fractions of methylcytosine nucleobases (e.g., 5mC and 5hmC). In the context of other methods (e.g., base editing) this treatment can be used to remove nucleic acid containing unwanted conversions of unmodified C to T.

Activation-induced cytidine deaminase (AID) is a deaminase that plays a critical role in the immune system by deaminating cytosine residues in DNA, which leads to mutations that change antibody affinities and classes. AID has been used in genome editing, particularly with CRISPR-Cas systems to introduce targeted point mutations in DNA (Berrios, K N et al., Cooperativity between Cas9 and hyperactive AID establishes broad and diversifying mutational footprints in base editors, Nucleic Acids Research, Volume 52, Issue 4, 28 Feb. 2024, Pages 2078-2090, https://doi.org/10.1093/nar/gkae024). Budzko et al. have reported a point mutation in hAID (N51) that decreases its ability to deaminate C to below detection level. N51 remained active on 5mC, whereas 5hmC is not deaminated by hAID or the N51 mutant. Budzko, L., Jackowiak, P., Kamel, K. et al. Mutations in human AID differentially affect its ability to deaminate cytidine and 5-methylcytidine in ssDNA substrates in vitro. Sci Rep 7, 3873 (2017)).

As used herein and in the appended claims, the singular forms “a”, “an”, and “the” include plural referents unless the context clearly dictates otherwise. For example, the term “a protein” refers to one or more proteins, i. e., a single protein and multiple proteins. Optional elements can be expressly excluded where exclusive terminology is used, such as “solely,” “only”, in connection with the recitation of the optional elements or when a negative limitation is specified.

Numeric ranges are inclusive of the numbers defining the range. All numbers should be understood to encompass the midpoint of the integer above and below the integer i. e., the number 2 encompasses 1.5-2.5. The number 2.5 encompasses 2.45-2.55 etc. When sample numerical values are provided, each alone can represent an intermediate value in a range of values and together can represent the extremes of a range unless specified.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that can be used in disclosed compositions, kits, and methods include HEPES, MES, MOPS, TAPS, tricine, and Tris. Additional examples of suitable buffering agents that can be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

In the context of the present disclosure, “methylcytosine-selective deaminase” means a hydrolase that deaminates modified cytosines, e.g., 5mC and/or 5hmC, each of which is also referenced herein as a type of methylcytosine nucleobase, preferentially relative to cytosines (also referred to as “unmodified cytosine” and “canonical cytosine”). A methylcytosine-selective deaminase can preferentially deaminate 5mC and 5hmC over C. In some cases, a methylcytosine-selective deaminase may preferentially deaminate 5mC over 5hmC and C or preferentially deaminate 5hmC over 5mC and C. A methylcytosine-selective deaminase can preferentially deaminate 5hmC over protected 5hmC, such as glucosylated 5hmC, 5caC, 5fC. In the context of the present disclosure, a methylcytosine-selective deaminase that preferentially deaminates 5mC and/or 5hmC relative to C in a DNA substrate has “methylcytosine-selective deaminase activity”.

As is described in Example 29, the amino acid sequences of methylcytosine-selective deaminases described herein are distributed among multiple clades of a phylogenetic tree, indicating that these enzymes have diverse amino acid sequences. Therefore, methylcytosine-selective deaminases useful in the deamination methods described herein do not necessarily have substantial sequence homology to each other.

As is described in Example 35, a species methylcytosine-selective deaminase can be characterized in that it contains a B5 family deaminase signature sequence. One signature sequence contains the following amino acids (positions are stated relative to SEQ ID NO:1 with a methionine (M) added to the N-terminus of SEQ ID NO:1): S at position 28; S at position 48; Y at position 51; G at position 53; and N at position 81, (SEQ ID NO:57) which can also be represented as S-[X]₂₀-S-xx-Y-x-G-[X]₂₈-N where x denotes any amino acid and where the number of amino acids between the defined amino acids may vary so long as the deaminase has methylcytosine-selective deaminase activity and retains at least three of the defined amino acids, at least four of the defined amino acids or the five defined amino acids.

Another methylcytosine-selective deaminase signature sequence contains (positions relative to SEQ ID NO:1 with a methionine (M) added to the N-terminus of SEQ ID NO:1), S at position 28; S at position 48; F at position 51; G at position 53; and N at position 81, (SEQ ID NO:56) which can also be represented as S-[X]₂₀-S-xx-F-x-G-[X]₂₈-N where x denotes any amino acid and where the number of amino acids between the defined amino acids may vary so long as the deaminase has methylcytosine-selective deaminase activity and retains at least three of the defined amino acids, at least four of the defined amino acids or the five defined amino acids.

As is described in Example 34, B5 deaminase family members are more broadly characterized in that the number of amino acids between S1 and S2 (in reference to the following simplified representation of the B5 deaminase family signature sequence: S1-X-S2-X-[Y or F]-X-G-X-N)) is up to 33 amino acids, and the number of amino acids between G and N is up to 66 (SEQ ID NOS: 58 and 59, for Y and F embodiments, respectively). Thus, a B5 deaminase signature sequence can have between 20-33 amino acids between S1 and S2, and between 28-66 amino acids between G and N, which could be represented as S-[X]₂₀₋₃₃-S-xx-F-x-G-[X]₂₈₋₆₆-N. It is expected that roughly 10% more or fewer amino acids in these variable regions is tolerated such that a B5 deaminase signature sequence could have between 20-33 amino acids between S1 and S2, and between 28-66 amino acids between G and N, which can be represented as S-[X]₁₇₋₃₆-S-xx-F-x-G-[X]₂₄₋₇₀-N.

Also encompassed are conservative substitutions of each methylcytosine-selective deaminase identified by SEQ ID or containing, where the substituted methylcytosine-selective deaminase has methylcytosine-selective deaminase activity. A methylcytosine-selective deaminase can be characterized in that its gene is located within a phage genome in proximity to thymidylate synthase or homolog thereof. As is described in Example 25, genomic DNA from a phage can be computationally screened for co-association within a neighborhood with a thymidylate synthase or homolog thereof. The phage genome can be from a phage suspected or known to have modified C.

A methylcytosine-selective deaminase can deaminate target mC with greater efficiency within particular sequence contexts. Table 10 of Example 34 shows sequence context preferences for several methylcytosine-selective deaminases. Thus, a methylcytosine-selective deaminase can be selected for targeting a particular context and two or more methylcytosine-selective deaminases can be combined to obtain desired deamination coverage for a particular experiment or workflow.

A methylcytosine-selective deaminase can be substantially inactive toward some forms of modified C. For example, Example 26 shows that B5 deaminase does not deaminate 5-formylC (5fC), 5-carboxyC (5caC) or glucosylated 5hmC (5ghmC).

A methylcytosine-selective deaminase can have one or more properties in addition to its deaminating activity that are useful in particular working contexts, such as stability in a desired temperature range and/or solubility, for example, in purified form. The methylcytosine-selective deaminases disclosed herein include methylcytosine-selective deaminases identified as SEQ ID NOS:1-40. Example 1 shows 5hmC and 5mC deamination activity of selected deaminases in vitro. Examples 2-6 show in vitro deamination activity of the B5, E10, C73 and PCBV1 deaminases.

The term “conservative substitution” means replacement of an amino acid in a polypeptide by one with similar characteristics; such substitutions are not likely to change the shape of the polypeptide chain, e.g., substituting one hydrophobic amino acid for another. For example, a non-polar amino acid (e.g., A, V, L, I, M, W, and F (and optionally C, G, and P)) can substitute for another non-polar amino acid, a polar amino acid (e.g., N, Q, S, T, and Y) can substitute for another polar amino acid (e.g., C, D, E, H, K, N, P. Q, R, S, and T), a positively charged amino acid (H, K, and R) can substitute for another positively charged amino acid, and a negatively charged amino acid (e.g., D and E) can substitute for another negatively charged amino acid. A substitute amino acid can be a natural amino acid (e.g., replacing another natural amino acid or a non-natural amino acid). A substitute amino acid can be a non-natural amino acid (e.g., replacing a natural amino acid or another non-natural amino acid).

In the context of the present disclosure, “DNA substrate” refers to a polynucleotide molecule suspected of containing, or known to contain, methylcytosine nucleobases (specifically 5mC and/or 5hmC) that optionally can be exclusively double-stranded, partially double-stranded and partially single-stranded, or exclusively single-stranded. The methylcytosine-selective deaminases described herein can deaminate methylcytosine (and to a lesser extent, cytosine) in the context of single stranded DNA substrate or trinucleotides. When using a DNA substrate is that is double-stranded, methods described herein involve treating the DNA substrate to partially or fully separate strands of the double-stranded DNA substrate to generate a DNA substrate comprising single stranded DNA.

In the context of the present disclosure, “double stranded” refers to any conformation of a polynucleotide in which two polynucleotide strands (e.g., separate molecules or spatially separated portions of a single molecule) are arranged anti parallel to one another in a helix with complementary bases of each strand paired with one another (e.g., in Watson-Crick base pairs). Duplex stability, in part, can be related to the ratio of complementary bases to mismatches (if any) in the two strands, ratio of pairs with three hydrogen bonds (e.g., G:C) to pairs with two hydrogen bonds (e.g., A:T, A:U) in the duplex, and the length of the strands with higher ratios and longer strands generally associated with higher stability. Duplex stability, in part, can be related to ambient conditions including, for example, temperature, pH, salinity, and/or the presence, concentration and identity of any buffer(s), denaturant(s) (e.g., formamide), crowding agent(s) (e.g., PEG), detergent(s) (e.g., SDS), surfactant(s), polysaccharide(s) (e.g., dextran sulfate), chelator(s) (e.g., EDTA), and nucleic acid(s) (e.g., salmon sperm DNA). Typical methods for strand separation include treatment with heat, salt, and/or chemical conditions. Examples include adding formamide or sodium hydroxide to a final concentration of about 20%, mixing, and incubating at 85 degrees C. for about 10 minutes for formamide or 85 degrees C. for about 10 minutes for sodium hydroxide, then placing the sample on ice. Therefore, a double-stranded DNA can be rendered single stranded or partially single stranded using a variety of well-known methods to produce a DNA substrate for deamination. A duplex polynucleotide can comprise one or more unpaired bases including, for example, a mismatched base, a hairpin loop, a single-stranded (5′ and/or 3′) end. Duplex polynucleotides (e.g., double-stranded DNA substrates) can have any desired length.

A DNA substrate can have a length of ≤50 nucleotides, 10-200 nucleotides, 80-400 nucleotides, 50-500 nucleotides, ≤500 nucleotides, ≤1 kb, ≤2 kb, ≤5 kb or ≤10 kb. Duplex polynucleotides can contain any desired number of mismatched or unpaired nucleotides, for example, ≤1 per 100 nucleotides, ≤2 per 100 nucleotides, ≤3 per 100 nucleotides, ≤5 per 100 nucleotides, or ≤10 per 100 nucleotides.

A DNA substrate can contain cytosines and at least one modified cytosine, e.g., 5mC, 5hmC, 5fC, 5CaC. Thus, a DNA substrate can be eukaryotic DNA (e.g., plant or animal) or bacterial DNA. A DNA substrate can be mammalian, e.g., from a human, such as cfDNA or DNA substrate from a biological sample. A DNA substrate can have a length suitable for the particular application (e.g., sequencing on different systems that accommodate certain lengths of nucleic acid; or base editing using different approaches). A sample comprising a DNA substrate used in a method described herein can also contain RNA. Whereas the methylcytosine-selective deaminases described herein have not shown selectivity for RNA or ribonucleotides, the RNA present in the sample will not be deaminated, facilitating some applications for the methylcytosine-selective deaminases.

A DNA substrate, or a deaminated product thereof, can comprise one or more adaptors, e.g., for ease of executing a workflow, such as for purification, for sample identification (e.g., barcodes and/or unique molecular identifiers), including unique molecular identifiers, for priming for sequencing and/or amplification, and for creating recognition sequences (e.g., for tagmentation, base editing), or any other purpose. As described in Example 10, such adaptors can contain modified nucleotides that are not deaminated during the methylcytosine deamination step. Additionally, adaptors containing modified nucleotides are not required, for example, when the adaptors are attached after the deamination step. A methylcytosine-selective deaminase described herein can be a fusion protein.

Methylcytosine (5mC and/or 5hmC) adjacent to guanines (CG context) in the DNA substrate can be deaminated by the methylcytosine-selective deaminases as well as, not as well as, or better than mCs in other sequence contexts (“CH” context, where H=A, C, T). Example 34 provides exemplary preferred and non-preferred sequence contexts for five methylcytosine-selective deaminases provided herein (G12, B5, E10, F7, H3) determined by sequencing of deaminated XP12 genomic DNA.

In the context of the present disclosure, “fusion protein” refers to a protein composed of two or more polypeptide components that are un-joined in their native state. Fusion proteins can be a combination of two, three or four or more different proteins. For example, a fusion protein can comprise two naturally occurring polypeptides that are not joined in their respective native states. A fusion protein can comprise two polypeptides, one of which is naturally occurring and the other of which is non-naturally occurring. A fusion protein can have one or more heterologous domains added to the N-terminus, C-terminus, and or the middle portion of the protein.

If two parts of a fusion protein are “heterologous”, they are not part of the same protein in its natural state. Examples of fusion proteins include methylcytosine-selective deaminases fused to a protein such as a DNA binding domain (e.g., the DNA binding domain of a transcription factor, a non-specific DNA-binding domain (e.g., Sso7d), or a specific DNA binding domain (e.g., BDO9; see, for example, U.S. Pat. No. 9,963,687); and enzyme (e.g., an endonuclease), an antibody, a binding domain suitable for immobilization such as maltose binding domain (MBP), a histidine tag (“His-tag”), a chitin binding domain, an alpha mating factor or a SNAP-Tag® (New England Biolabs, Ipswich, MA (see for example U.S. Pat. Nos. 7,939,284 and 7,888,090)), a methyl binding domain (MBD), with the deaminase optionally positioned closer to the N-terminus or closer to the C-terminus than the other component(s). A binding peptide can be used to improve solubility or yield of the deaminase during the production of the protein reagent. Other examples of fusion proteins include fusions of a deaminase and a heterologous targeting sequence, a linker, an epitope tag, a detectable fusion partner, such as a fluorescent protein, β-galactosidase, luciferase and/or functionally similar peptides. Components of a fusion protein can be joined by one or more peptide bonds, disulfide linkages, and/or other covalent bonds.

In some embodiments, the DNA substrate can be genomic DNA, organelle DNA, cDNA, or other DNAs of interest and can be or arise from any desired source (e.g., human, non-human mammal, plants, insects, microbial, viral, or synthetic DNA), or a fragment thereof. A DNA substrate can be prepared, in some embodiments by extracting (e.g., genomic DNA) from a biological sample and, optionally, fragmenting it. Fragmenting DNA can comprise mechanically fragmenting the DNA (e.g., by sonication, nebulization, or shearing) or enzymatically fragmenting the DNA. Examples of enzymes for fragmentation include NEBNext® Fragmentase®, Ultrashear, and FS systems (New England Biolabs, Ipswich MA)), among others. In some embodiments, DNA for deamination can already be fragmented (e.g., as is generally the case for FFPE samples and circulating cell-free DNA (cfDNA)).

Methylcytosine nucleobases can be present in a variety of types of DNA substrate samples. For example, the presence, absence, or amount of modified 5mC and/or 5hmC can be analyzed in forensic casework to help identify individuals, determine the age of biological samples, and even predict certain phenotypic traits. For example, DNA methylation patterns can be used to distinguish between different types of body fluids at a crime scene. In environmental samples, modified cytosines can be used to monitor the effects of pollutants and other environmental factors on organisms. These modifications can serve as biomarkers for exposure to certain chemicals or environmental stressors. Ancient DNA often contains deaminated cytosines, which can be used to infer methylation patterns. Methylated cytosines deaminate to thymine, while unmethylated cytosines deaminate to uracil. In plant and animal samples, detecting modified cytosines can be used to understand gene expression in response to various environmental conditions (e.g., stress responses and disease states) and development stages. DNA methylation patterns are associated with various diseases, including cancers, neurological disorders, and cardiovascular diseases. Detecting these patterns can help in early diagnosis, monitoring disease progression and predicting outcomes. Thus, in particular embodiments, the DNA substrate comprises DNA selected from genomic DNA, ancient DNA, forensic DNA, environmental DNA, human DNA, cell free DNA, synthetic DNA, and plant DNA. In an embodiment, the DNA substrate can comprise tumor DNA.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition can differ from naturally occurring polynucleotides polypeptides, carbohydrates, lipids, or compositions in one or more respects. For example, a polymer (e.g., a polynucleotide, polypeptide, or carbohydrate) can differ in the kind and arrangement of the component building blocks (e.g., nucleotide sequence, amino acid sequence, or sugar molecules). A polymer can differ from a naturally occurring polymer with respect to the molecule(s) to which it is linked. For example, a “non-naturally occurring” protein can differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid can contain one or more other modifications (e.g., an added label or other moiety) to the 5′- end, the 3′ end, and/or between the 5′- and 3′-ends (e.g., methylation) of the nucleic acid. A “non-naturally occurring” composition can differ from naturally occurring compositions in one or more of the following respects: (a) having components that are not combined in nature; (b) having components in concentrations not found in nature; (c) omitting one or components otherwise found in naturally occurring compositions; (d) having a form not found in nature, e.g., dried, freeze dried, crystalline, aqueous; and (e) having one or more additional components beyond those found in nature (e.g., buffering agents, a detergent, a dye, a solvent or a preservative).

With reference to an amino acid, “position” refers to the place such amino acid occupies in the primary sequence of a peptide or polypeptide numbered from its amino terminus to its carboxy terminus. A position in one primary sequence can correspond to a position in a second primary sequence, for example, where the two positions are opposite one another when the two primary sequences are aligned using an alignment algorithm (e.g., BLAST (Journal of Molecular Biology. 215 (3): 403-410) using default parameters (e.g., expect threshold 0.05, word size 3, max matches in a query range 0, matrix BLOSUM62, Gap existence 11 extension 1, and conditional compositional score matrix adjustment) or custom parameters). An amino acid position in one sequence can correspond to a position within a functionally equivalent motif or structural motif that can be identified within one or more other sequence(s) in a database by alignment of the motifs. Analogously, with reference to a nucleotide, “position” refers to the place such nucleotide occupies in the nucleotide sequence of an oligonucleotide or polynucleotide numbered from its 5′ end to its 3′ end.

The present disclosure relates to naturally occurring and non-naturally occurring methylcytosine-selective deaminases. A non-naturally occurring methylcytosine-selective deaminase can be similar to, but differ from, a naturally occurring protein. Non-naturally methylcytosine-selective deaminases can be, for example, truncated versions of a naturally-occurring protein, in which cases, the non-naturally occurring methylcytosine-selective deaminase can have a high degree of identity to a portion of a naturally-occurring sequence, but lack, for example, structural and/or functional domains or sub-units of the corresponding naturally-occurring proteins. A non-naturally occurring methylcytosine-selective deaminase can have any number of insertions, deletions, or substitutions relative to a naturally occurring enzyme. For example, a non-naturally occurring methylcytosine-selective deaminase can have less than 100% identity, less than 99% identity, less than 98% identity, less than 90% identity, less than 85% identity, less than 80% identity, less than 70% identity, less than 60% identity, less than 50% identity, less than 40% identity, less than 30% identity, or less than 20% identity to a naturally occurring enzyme. Non-naturally occurring methylcytosine-selective deaminases can include expression and/or purification tags. Non-naturally occurring methylcytosine-selective deaminase disclosed herein can have an amino acid sequence that is at least 80% identical (e.g., at least 90% identical, at least 95% identical or at least 98% identical or at least 99% identical to) the C-terminal deaminase domain of a naturally-occurring protein, wherein the methylcytosine-selective deaminase possesses a DNA deaminase activity and does not comprise the N-terminus of the corresponding naturally-occurring protein (if any). In some embodiments, a non-naturally occurring methylcytosine-selective deaminase lacks at least 10, at least 20, at least 50 or at least 100 of the N-terminal amino acids of the corresponding naturally-occurring protein. In some embodiments, a methylcytosine-selective deaminase is no more than 128 amino acids in length, e.g., no more than 100 amino acids in length, no more than 110 amino acids in length, no more than 150 amino acids in length, or more than 200 amino acids in length. Non-naturally occurring methylcytosine-selective deaminases can be engineered using various approaches such as ancestral reconstruction, mutagenesis, rational design and directed evolution. Four engineered methylcytosine-selective deaminases described herein are SEQ ID NOs:48, 49, 50 and 51. Using the CE assay described herein below with a DNA substrate containing C and 5mC, each of these deaminases had activity equivalent to that of the B5 deaminase (SEQ ID NO:1).

According to some embodiments, a methylcytosine-selective deaminase can comprise an amino acid sequence having at least 80%, at least 85%, at least 88% identical, at least 90%, at least 92%, at least 93%, at least 95%, at least 96%, at least 97%, at least 98% or at least 99% identity to any of SEQ ID NOS:1-40, 54 and 55. In some embodiments, a methylcytosine-selective deaminase can be encoded by a nucleic acid sequence that, when transcribed, translated, and/or processed, results in an amino acid sequence having at least 80%, at least 85%, at least 90%, at least 93%, at least 96%, at least 97%, at least 98% or at least 99% identity to any of SEQ ID NOS:1-40, 54 and 55. A methylcytosine-selective deaminase can have an amino acid sequence at least 90% (e.g., at least 95%, at least 98%, at least 99%) identical to any of SEQ ID NOS: 1-40, 54 and 55. In some embodiments, a methylcytosine-selective deaminase can have an amino acid sequence identical to any of SEQ ID NOS: 1-40, 54 and 55. In some embodiments, a non-naturally occurring methylcytosine-selective deaminase lacks a segment of its corresponding naturally-occurring protein, for example, at least 10, at least 20, at least 50 or at least 100 of the N-terminal or C-terminal amino acids. Variants can be designed using sequence alignments and structural information. In some embodiments, a methylcytosine-selective deaminase can contain a fragment of a wild type protein, where the fragment contains a deaminase domain, but lacks other domains of the wild type protein that can be C-terminal and/or N-terminal to the deaminase domain.

In an embodiment, a methylcytosine-selective deaminase provided herein comprises a polypeptide that is capable of deaminating 5-methyl cytosine (5mC) to thymidine (T) and/or 5-hydroxylmethyl cytosine (5hmC) to hydroxymethyluridine (hmU); and preferentially deaminates 5mC and/or 5hmC relative to cytosine (C); wherein the polypeptide is selected from: a non-natural fusion protein comprising a B5 deaminase family signature sequence; a non-natural fusion protein comprising a methylcytosine-selective deaminase encoded by a viral gene located in a viral genome in proximity to a thymidylate synthase gene; a non-natural fusion protein comprising an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 53, 54 and 55; and a polypeptide comprising an amino acid sequence selected from SEQ ID NOS: 37, 38, 39, and 40.

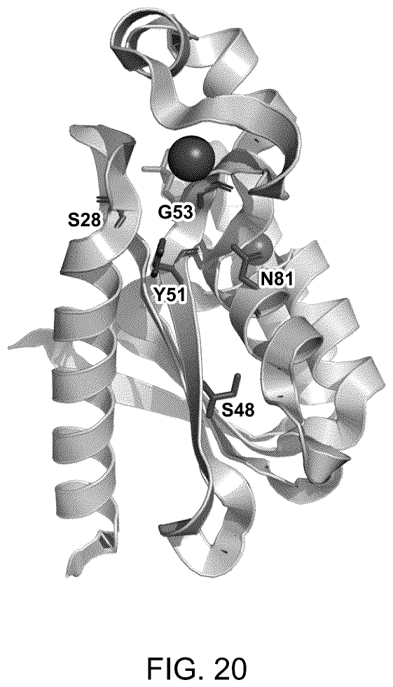

An exemplary methylcytosine-selective deaminase described herein as B5 deaminase (SEQ ID NO:1) is a single domain protein, consisting of 154 amino acids in length with a predicted molecular mass of 17.04 kDa. An AlphaFold2 predicted structure revealed a prototypical mononucleotide deoxycytidine deaminase fold. Its closest structural homolog in PDB found by Foldseek is a cytidine deaminase from the Chlorella virus PBCV-1. FIG. 20 shows the predicted B5 deaminase structure, highlighting a methylcytosine-selective deaminase signature sequence.

In some embodiments, a methylcytosine-selective deaminase can be a fusion protein. For example, a methylcytosine-selective deaminase can have a purification tag (e.g., a His-tag or the like) at either end. In some embodiments, a methylcytosine-selective deaminase can be fused to a DNA binding protein (e.g., the DNA binding domain of a transcription factor) or the protein component of a nucleic acid-guided endonuclease (e.g., a catalytically dead Cas9 (dCas9) or a Cas9 nickase (nCas9) or TALEN (transcription activator-like effector nucleases)) so that the fusion protein can affect site-specific C to T substitutions in a genome. Example methods of “base editing” are described in, for example, Komor et al), among other publications. An example of a B5 deaminase-His-tag fusion protein is provided as SEQ ID NO:9.

Provided herein are methods for sequencing DNA substrates to identify epigenetic modifications such as 5mC and 5hmC independently, and 5mC and 5hmC in a pool. In an embodiment, the methods involve contacting a DNA substrate comprising at least one modified cytosine (C) with a methylcytosine-selective deaminase to produce a deamination product, wherein the methylcytosine-selective deaminase is capable of converting 5-methylcytosine (5mC) to thymidine (T) and/or 5-hydroxymethylcytosine (5hmC) to 5-hydroxymethyl uridine (5hmU); sequencing the deamination product, or amplifying the deamination product to produce amplification products, and sequencing the amplification products, in each case, to produce sequence reads. As would be expected by those of skill in the art, it is understood that a portion of the deamination product or a portion of the amplification products can yield sufficient information. Therefore, sequencing the deamination product or the amplification product encompasses sequencing a portion of these materials.

Also provided herein is a sequencing method, involving (a) contacting a DNA substrate comprising cytosine (C) and at least one methylcytosine nucleobase selected from 5-methylcytosine (5mC) and 5-hydroxymethylcytosine (5hmC) or comprising C and both 5mC and 5hmC, with a methylcytosine-selective deaminase to produce a deamination product, wherein the methylcytosine-selective deaminase (i) is capable of deaminating 5mC to thymidine (T) and/or 5hmC to hydroxymethyluridine (hmU) and (ii) preferentially deaminates the at least one methylcytosine nucleobase relative to cytosine (C); and (b) sequencing the deamination product, or amplifying the deamination product to produce an amplification product, and sequencing the amplification product, in each case, to produce sequence reads, wherein positions of Cs and position of the at least one methylcytosine nucleobase in the DNA substrate is determined based on the sequence reads.

In embodiments described herein, the methods employ a methylcytosine-selective deaminase that preferentially deaminates methylcytosine (mC) relative to cytosine (C). Such a methylcytosine-selective deaminase can be a natural deaminase, such as a bacteriophage (also referred to as “phage”) deaminase or viral deaminase described herein, or non-natural modification thereof (e.g., a fusion protein, engineered polymer (e.g., generated using a variety of well-known methods such as in silico ancestral reconstruction (see, for example, Merkl R, Sterner R. Ancestral protein reconstruction: techniques and applications. Biol Chem. 2016 January; 397(1):1-21. doi: 10.1515/hsz-2015-0158. PMID: 26351909). Exemplary methylcytosine-selective deaminases have an amino acid sequence that is at least 90% identical to any of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55.

Provided herein is a sequencing method, involving (a) contacting a DNA substrate comprising cytosine (C) and at least one methylcytosine nucleobase selected from 5-methylcytosine (5mC) and 5-hydroxymethylcytosine (5hmC) or comprising C and both 5mC and 5hmC, with a methylcytosine-selective deaminase to produce a deamination product, wherein the methylcytosine-selective deaminase (i) is capable of deaminating 5mC to thymidine (T) and/or 5hmC to hydroxymethyluridine (hmU) and (ii) preferentially deaminates the at least one methylcytosine nucleobase relative to cytosine (C); and (b) sequencing the deamination product, or amplifying the deamination product to produce an amplification product, and sequencing the amplification product, in each case, to produce sequence reads, wherein positions of Cs and position of the at least one methylcytosine nucleobase in the DNA substrate is determined based on the sequence reads.

In embodiments of the methods described herein, the DNA substrate contains or is suspected of containing methylcytosine. In an embodiment, the DNA substrate comprises genomic DNA. The DNA substrate can also contain a copied strand, for example, as described herein in reference to multi-copy strand workflows described herein.

In embodiments of the methods described herein, the DNA substrate comprises DNA that has been pretreated to protect one or more nucleobases such as 5hmC. Such pre-treatment for 5hmC can therefore be performed with any of a variety enzymes such as DNA beta-glucotransferase and/or carbamoyl transferase, with a S-adenosyl-L-methionine analog paired with an engineered methyltransferase (Wang, T., Fowler, J. M., Liu, L. et al. Direct enzymatic sequencing of 5-methylcytosine at single-base resolution. Nat Chem Biol 19, 1004-1012 (2023). https://doi.org/10.1038/s41589-023-01318-1), a glycotransferase or a chemical treatment or combination. Pre-treatment of DNA substrate to protect C can employ an enzyme such as 4mC methylase and/or N4 methylase. As is described herein, methylcytosine-selective deaminases described herein can have the characteristic of insignificant deamination of C having modification at the 4 position. Therefore, pretreatment of DNA substrate with an enzyme that modifies the 4 position of C would prevent unwanted background deamination of C when using a methylcytosine-selective deaminase that has an undesired activity toward C.

In an embodiment, a DNA substrate does not contain DNA that has been pre-treated with a TET methylcytosine dioxygenase. In an embodiment, a DNA substrate contains DNA that has been pre-treated with a TET methylcytosine dioxygenase. Pre-treatment with a TET methylcytosine dioxygenase can be used, for example, when the selected methylcytosine-selective deaminase has a preference for deaminating 5hmC. By converting 5mC to 5hmC, such a deaminase is useful for detecting both 5mC and 5hmC. Pre-treatment can therefore comprise incubation with an enzyme. Pre-treatment can also comprise treatment with a chemical reagent that protects one or more nucleobase. For example, after enzymatic treatment with a TET, 5mC and 5hmC are converted to 5caC. pyridine borate can be used to reduce 5caC to dihydrouracil, which is subsequently converted to T during PCR amplification.

In an embodiment, prior to step (b), the DNA substrate is contacted with a second deaminase having a different specificity from the methylcytosine-selective deaminase of (a).

It can be desirable to cleave Us that are generated by background C-to-U deamination activity of a methylcytosine-selective deaminase described herein. Cleavage of Us can result in fragmentation of the U-containing DNA substrate so that intact C-containing DNA substrate can be the more dominant species undergoing analysis. Therefore, in some embodiments, the methods can involve, before sequencing, contacting the deamination product with a substance that generates a single nucleotide gap at the location of a uracil residue, such as USER enzyme or UDG.

In some embodiments, the DNA substrate is treated with more than one deaminating agent in addition to the methylcytosine-selective deaminases described herein. The deaminating agent can be another enzyme (e.g., well known enzymes such as APOBEC family deaminases, and those described in WO2023097226) or an abiotic method such as a chemical method (e.g., bisulfite). The deaminating agent is selected based on the desired detection scheme, e.g., sequential conversion of modified Cs with detection of nucleobases after each conversion, or bulk conversion of modified Cs, using a bioinformatics schema that reveals the position of Cs/modified Cs as needed for a particular application.

As is described herein, a DNA substrate can be prepared using a multi-copy strand process. Therefore, in an embodiment, a method can involve (a) ligating a hairpin adaptor to a double-stranded genomic DNA sample to produce a ligation product; (b) enzymatically generating a free 3′ end in a double-stranded region of the hairpin adaptor in the ligation product; (c) extending the free 3′ end in a reaction mix that comprises a strand-displacing or nick-translating polymerase, dGTP, dATP, dTTP and modified dCTP, wherein the modified dCTP is deamination resistant to produce and extended product; to produce a single-stranded DNA substrate. Modified dCTP forms that are deamination resistant include, for example, pyrrolo-CTP and N⁴-mCTP.

A method can include mapping sequencing reads to a reference sequence. The reference sequence can be obtained from a public database; can be obtained by sequencing the same sample, for example, when the DNA sample to be sequenced includes ‘genomic’ and ‘epigenomic’ copies of sequence; can be obtained by sequencing another sample, including a sample pool; or can be computationally determined.

A method can include preparing a DNA substrate or other DNA product by polishing DNA ends (e.g., the ends of fragmented DNA). For example, DNA ends can be contacted with (a) a proofreading polymerase to excise 3′ overhanging nucleotides, if any, (b) a proofreading and/or non-proofreading polymerase to fill in 5′ overhangs, if any, and/or (c) a polynucleotide kinase (PNK) to phosphorylate unphosphorylated 5′ ends, if any. In some embodiments, a method can comprise contacting DNA ends (e.g., blunt ends) with a non-proofreading polymerase to add an untemplated A-tail (e.g., a single base overhang comprising adenine) to the 3′ end. Methods can include, according to some embodiments, ligating one or more adaptors to DNA ends. Adaptors can comprise one or more sample tags, unique molecular identifiers (UMIs), modified nucleotides, primer sequences (e.g., for sequencing). In some embodiments, adaptors can comprise cytosines (or adenines) that are not substrates for the deaminase to be used. If desired, polishing products and/or ligation products can be cleaned up, for example, to separate polishing products or ligation products, as applicable, from enzymes, unreacted nucleotides and/or adaptors. In some embodiments, a method can include contacting a DNA substrate and an enzyme that protects a modified cytosine from deamination (e.g., carbamoyl transferase, glucosyltransferase (e.g., a BGT to protect 5hmC), or glycosyltransferase) to produce a modified deaminase substrate to be subsequently treated with a methylcytosine-selective deaminase described herein. This is useful for example, in the case of 5hmC as described in, for example, in Examples 8, 9, and 10.

In some embodiments, a method can further involve amplifying the deamination product to produce an amplification product, thereby copying any deaminated modified 5mCs in the original strand to Ts in the amplification product. The amplification can be performed using polymerase chain reaction or an isothermal method. When performing polymerase chain reaction, any of a variety of well-known polymerases can be used. Exemplary polymerases used in the Examples herein include 5Q and 5QU.

Deamination methods can further comprise ligating an adaptor to the DNA substrate prior to amplification or to the deaminated product, and amplifying the adaptor-ligated deaminated product using primers complementary to sequences in the adaptor. Any of a variety of adaptors can be used (e.g., adaptors designed for a function such priming and/or capture, and those designed for particular sequencing workflows, including asymmetric (or “Y”) adaptors, e.g., an Illumina P5/P7 adaptors).

In some embodiments, a method can further include a target enrichment step during preparation of the deamination product or amplification product. In some embodiments, a method can involve sequencing a deamination product or amplification product, in each case, to produce sequence reads. Deamination products and/or amplification products can be sequenced using any suitable system including systems of Illumina, PacBio, Oxford Nanopore, Ion Torrent, Roche, Element Biosciences, Complete Genomics, and Singular Genomics.

In some embodiments, a deaminated product can be sequenced directly, without amplification, if desired, for example, by Nanopore, PacBio sequencing or short read sequencing. A sequencing step can result in at least 10,000, at least 100,000, at least 500,000, at least 1M, at least 10M, at least 100M, at least 1B or at least 10B sequence reads per reaction. In some cases, the reads can be paired-end reads. A method can involve analyzing sequence reads to identify a 5mC in the DNA substrate, where a 5mC can be identified as a “T” because it is deaminated by a methylcytosine-selective deaminase described herein. A method can involve analyzing sequence reads to identify a 5mC or 5hmC in the DNA substrate, where a 5mC (5mC conversion to T) or 5hmC (5hmC conversion 5hmU) to can be identified as a “T” because it is deaminated by a methylcytosine-selective deaminase described herein.

In specific embodiments, the methods described herein can be used for detecting 5mC in a DNA substrate. In an embodiment, the method involves contacting a DNA substrate comprising at least one modified cytosine (C) with a methylcytosine-selective deaminase to produce a deamination product, wherein the methylcytosine-selective deaminase is capable of converting 5-methylcytosine (5mC) to thymidine (T) and 5-hydroxymethylcytosine (5hmC) to 5-hydroxymethyl uridine (5hmU) to produce a deamination product; contacting the deamination product with a substance that generates a single nucleotide gap at the location of a uracil residue, such as USER enzyme or UDG; sequencing the non-fragmented deamination product, or amplifying the non-fragmented deamination product to produce amplification products, and sequencing the amplification products, in each case, to produce sequence reads.

In other specific embodiments, the methods described herein can be used for detecting 5hmC in a DNA substrate. In an embodiment, the method involves (a) treating a DNA substrate comprising at least one modified cytosine (C) with a treatment that protects 5hmC to produce a protected DNA substrate; (b) contacting the protected DNA substrate with a methylcytosine-selective deaminase, wherein the methylcytosine-selective deaminase is capable of converting 5-methylcytosine (5mC) to thymidine (T) and 5-hydroxymethylcytosine (5hmC) to 5-hydroxymethyl uridine (5hmU), and a deaminase capable of converting cytosine (C) to uracil (U), and to produce a deamination product; sequencing the deamination product, or amplifying the deamination product to produce amplification products, and sequencing the amplification products, in each case, to produce sequence reads, wherein 5mC is detected as T, 5hmC is detected as C, and C is detected as U (T in the sequencing reads).

In an embodiment, the methods can be used for detecting 5mC and 5hmC. A method for detecting 5mC and 5hmC. In an embodiment, the method involves (a) contacting a DNA substrate comprising at least one modified cytosine (C) with a methylcytosine-selective deaminase to produce a deamination product, wherein the methylcytosine-selective deaminase is capable of converting 5-methylcytosine (5mC) to thymidine (T) and 5-hydroxymethylcytosine (5hmC) to 5-hydroxymethyl uridine (5hmU) to produce a first deamination product; (b) optionally contacting the first deamination product with a substance that generates a single nucleotide gap at the location of any uracil residue (e.g., USER enzyme or UDG); (c) ligating a hairpin adaptor to the first deamination product to produce a ligation product; (d) enzymatically generating a free 3′ end in a double-stranded region of the hairpin adaptor in the ligation product; (e) extending the free 3′ end in a reaction mix that comprises a strand-displacing or nick-translating polymerase, dGTP, dATP, dTTP and modified d5mCTP, to produce an extended product; (f) deaminating the extended product using a deamination reagent that is not selective for 5mC to produce a second deamination product; (g) sequencing the second deamination product, or amplifying the second deamination product to produce amplification products, and sequencing the amplification products, in each case, to produce sequence reads.

Exemplary deamination reagents that do not deaminate 5mC include, e.g., bisulfite, a deaminase, such as those having appropriate selectivity described in WO2023097226, which is incorporated herein by reference.

As described in Example 7 and other examples, a DNA substrate can be subjected to deamination prior to or after steps such as end repair/dA-tailing and/or adaptor ligation. A DNA substrate can be prepared by pre-treatment with a methylcytosine protecting reagent, e.g., DNA beta-glucosyltransferase, carbamoyl transferase, glycosyltransferase, or a chemical treatment to convert the 5hmC in the starting DNA to forms resistant to a methylcytosine-selective deaminase.

In some embodiments, the methylcytosine-selective deaminase can be used in a workflow for simultaneously obtaining sequence for one more genetic bases (e.g., G, A T, C) and epigenetic bases (e.g., methylated bases), for example to better distinguish between genetic C-to-T mutations and epigenetically modified cytosine. These workflows (e.g., Yan et al, Genome Res. 2022; gr. 277080.122, and Füllgrabe, et al., Nat Biotechnol (2023). https://doi.org/10.1038/s41587-022-01652-0) involve copying strands of genomic DNA fragments to generate two copies of the strand on a single DNA strand. The multi-copy strands are then deaminated, followed by sequencing. This results in obtaining two sequence reads of the same strand of the genomic DNA fragment (and if desired four reads because both strands of the genomic DNA fragment can be subjected to this process); bioinformatics tools are used to discern whether Cs arose from modified C or from a mutation in the genomic DNA, as well as to identify errors arising from sequencing or amplification steps. Such workflows involve linking together the two strands of the genomic DNA, e.g., using a hairpin; breaking that linkage to synthesize the copy, thereby creating the multi-copy strand. Then, in either order, deaminating the multi-copy strand, and adding sequencing primers to the multi-copy strands to obtain reads of the original and copied sequences. The sequences are determined using rules based on the selected deamination process. For example, in some embodiments, the DNA substrate can be made by ligating a hairpin adaptor to a double-stranded fragment of DNA to produce a ligation product, enzymatically generating a free 3′ end in a double-stranded region of the hairpin adaptor in the ligation product, and extending the free 3′ end in a reaction mix that comprises a strand-displacing or nick-translating polymerase, dGTP, dATP, dTTP and a modified deamination-resistant CTP. In this method, the modified deamination-resistant CTP is incorporated into the new strand. In another embodiment, the extending of the free 3′ end is performed in a reaction mix that comprises a strand-displacing or nick-translating polymerase, dGTP, dATP, dTTP and d5mCTP. The extended strand can then optionally be deaminated using a method not selective for 5mC as described in Example 11.

A methylcytosine-selective deaminase can be used for base editing. In one embodiment, the method involves contacting a fusion protein with a target sequence to produce an edited target sequence comprising at least one 5mC or 5hmC, wherein the fusion protein comprises a methylation-selective deaminase fused to a DNA binding domain. In an embodiment, the DNA binding domain is selected from a Cas9 domain, a Cas12 domain, a transcription activator-like effector nuclease (TALEN domain), a zinc finger (ZF) domain, a transcription activator-like effector (TALE) domain, and a methyl binding domain (MBD) domain. In an embodiment the fusion protein includes a guide RNA complementary to at least a portion of the targeted sequence. In an embodiment, the fusion protein comprises an enzyme that is at least 90% identical to any of SEQ ID NOS:1-51, such as an enzyme that is at least 90% identical to SEQ ID NO:1. In an embodiment the fusion protein comprises an enzyme that is identical to any of SEQ ID NOS:1-51, and in an embodiment the fusion protein comprises an enzyme that is identical to SEQ ID NO:1. Example methods of “base editing” are described in, for example, Komor et al (Nature 533: 420-424), among other publications.

In some embodiments, a DNA substrate can be treated with a DNA glycosylase such as hSMUG1 (human Single-strand-selective Monofunctional Uracil-DNA Glycosylase 1). Such a glycosylase enzyme will recognize and excise 5hmU bases. This process initiates base excision repair (BER), leading to the removal of 5hmU and the creation of abasic sites.

The methods for deaminating a DNA substrate using a methylcytosine-selective deaminase described herein can also be used for the temporally controlled introduction of dTTP into an in vitro reaction through the enzymatic conversion of a 5mdCTP pool to the thymidine triphosphate form. The methods for deaminating a DNA substrate using a methylcytosine-selective deaminase described herein can additionally be used for the temporally controlled formation of a poly dT tail by the incorporation of 5mdC via terminal transferase (TdT) and subsequent deamination to poly dT, to hybridize to a poly dA immobilized on magnetic beads.

The present disclosure relates, in some embodiments, to a kit comprising a methylcytosine-selective deaminase. A methylcytosine-selective deaminase kit can include, for example, one or more methylcytosine-selective deaminases and, optionally, any of the following: one or more enzymes that alter the deamination susceptibility of one or more modified cytosines (e.g., a TET methylcytosine dioxygenase and/or a DNA beta-glucosyltransferase); one or more other enzymes (e.g., another deaminase, a ligase, a polymerase, a proteinase); a buffer (which optionally can be in concentrated form, and which can include one or more additives (e.g., glycerol); one or more salts (e.g. KCl); one or more reducing agents; one or more chelating agents (e.g., EDTA); one or more detergents; one or more non-ionic surfactants; one or more ionic (e.g. anionic or zwitterionic) surfactants; one or more crowding agents; one or more antibodies (e.g., modification specific antibodies); one or more nucleic acids (e.g., primer, hairpin, adaptor). A kit can include dNTPs, such as one, two, three of all four of dATP, dTTP, dGTP, dCTP. A kit can include one or more modified nucleotides, such as a modified dCTP (e.g., 5hmdCTP, 5fdCTP, 5cadCTP, 5mdCTP, pyrrolo-dCTP and N4mdCTP).

The provided kits can be used for deaminating DNA substrates for a variety of purposes, such as sequencing and base editing. In an embodiment, a kit includes: (a) a methylcytosine-selective deaminase capable of converting 5-methylcytosine (5mC) to thymidine (T) and 5-hydroxymethylcytosine (5hmC) to 5-hydroxymethyl uridine (5hmU); and (b) one or more reagents selected from an enzyme (e.g., DNA polymerase, DNA beta-glucosyltransferase, a deaminase of different specificity from the deaminase of (a)) a primer, an adaptor.

One or more components of a kit can be included in one container, or components can be in different containers. The kit can be configured for one-step use or for parallel or sequential steps. For example, a kit can comprise two components in a single tube (e.g., a deaminase and a storage buffer) and all other components in separate, individual tubes, in each case, with the contents provided in any desired form (e.g., liquid, dried, lyophilized). One tube in a kit can contain a master mix, for example, for receiving and amplifying a DNA (e.g., a deaminated DNA). For example, a deaminase can be deposited in the cap of a tube while components for transcribing a template nucleic acid are deposited in the body of the tube.

In some embodiments, a kit can further include (a) a TET methylcytosine dioxygenase (e.g., TET2) and a DNA beta-glucosyltransferase or (b) a TET methylcytosine dioxygenase and no DNA beta-glucosyltransferase. In some embodiments, a kit does not contain either a TET methylcytosine dioxygenase or DNA beta-glucosyltransferase. In some embodiments, a kit further comprises a modified dCTP selected from 5hmdCTP, 5fdCTP, 5cadCTP, 5mdCTP, pyrrolo-dCTP and N4mdCTP. In some embodiments, a kit can additionally comprise a ligase, a polymerase, a proteinase K, which can be a thermolabile proteinase K.

A provided methylcytosine-selective deaminase can be lyophilized or in a storage solution; other components of a kit can also be lyophilized or in a storage solution. A methylcytosine-selective deaminase can be present on a solid support, such as a bead, plate, membrane, slide, tube, strip, cartridge, device, and the like, optionally together with one or more other substances. Example 24 describes use of a methylcytosine-selective deaminase linked to a solid support. Other components of a kit can be provided on such a solid support (or kit components can be on a separate solid support if desired for a particular application). A solid support can be constructed of any of a variety of materials, such as glass, plastic, natural or man-made fiber, ceramic, paper, nitrocellulose, metal, or a combination, which will generally depend on the application.

The present disclosure provides methylcytosine-selective deaminase compositions including, for example, reaction mixtures. According to some embodiments, methylcytosine-selective deaminase compositions can comprise a methylcytosine-selective deaminase and a DNA substrate. A methylcytosine-selective deaminase composition can comprise, for example, a deaminase variant (e.g., having an amino acid sequence at least 90% identical to one or more of SEQ ID NOS:1-51). A methylcytosine-selective deaminase composition can be free of one or more other catalytic activities. For example, such a composition can be free of nucleases that cleave DNA, free of polymerase activity, and/or free of protease activity, in each case, under desired test conditions (e.g., conditions of time, temperature, pH, salinity, model substrate and/or others), for example, conditions intended to replicate conditions of a specific use of methylcytosine-selective deaminase composition or intended to represent conditions for a range of uses. The reaction mixture can additionally contain one or more of a DNA beta-glucosyltransferase, glycosyltransferase, a carbamoyl transferase, TET methylcytosine dioxygenase (e.g., TET2), another deaminase (including another methylcytosine-selective deaminase) a ligase, a polymerase, a proteinase K, and/or a thermolabile proteinase K.

In some embodiments, methylcytosine-selective deaminases and compositions comprising one or more deaminases can have any desirable form including, for example, a liquid, a gel, a film, a powder, a cake, and/or any dried or lyophilized form. A methylcytosine-selective deaminase composition can further comprise a support, for example, a film, gel, fabric, porous material, matrix, or bead. Such support can be, for example, a magnetic material, agarose, a plastic (e.g., polystyrene, polyacrylamide) and/or chitin. Such a support can further contain a desired surface chemistry, a ligand, a tag (e.g., for purification or detection), or other substance useful for a particular application.

According to some embodiments, a methylcytosine-selective deaminase composition can include a methylcytosine-selective deaminase and, optionally, any of (including one or more of): a buffering agent (e.g., a storage buffer, a reaction buffer), a salt (e.g., NaCl, MgCl₂, CaCl₂), a protein (e.g., albumin; an enzyme, such as a DNA beta-glucosyltransferase), a stabilizer, a detergent (for example, ionic, non-ionic, and/or zwitterionic detergents (e.g., octoxinol, polysorbate 20)), a poloxamer, a polynucleotide (e.g., an oligonucleotide), a denaturant, an unwinding agent (e.g., a helicase), a cell (e.g., cultured cell or extract thereof), a biological sample (e.g., tissue, blood or a fraction thereof), an aptamer, a crowding agent, a sugar (e.g., a mono, di, tri, tetra, or higher saccharide), a starch, a cellulose, a glass-forming agent (e.g., for lyophilization), a lipid, an oil, aqueous media, a support (e.g., a bead, tube, strip, membrane) and/or (non-naturally occurring) combinations thereof. Combinations can include for example, two or more of the listed components (e.g., a buffering agent and a protein) or a plurality of a single listed component (e.g., two different buffering agents or two different proteins).

All publications, patents, and patent applications mentioned in this specification are herein incorporated by reference to the same extent as if each individual publication, patent, or patent application was specifically and individually indicated to be incorporated by reference. Reagents referenced in this disclosure can be made using available materials and techniques, obtained from the indicated source, and/or obtained from New England Biolabs, Inc. (Ipswich, MA).

### EMBODIMENTS

Embodiment 1. A method, comprising:
(a) contacting a DNA substrate comprising at least one modified cytosine (C) with a methyl cytosine-selective deaminase to produce a deamination product, wherein the methyl cytosine-selective deaminase is capable of converting 5-methylcytosine (5mC) to thymidine (T);
(b) sequencing the deamination product, or amplifying the deamination product to produce amplification products, and sequencing the amplification products, in each case, to produce sequence reads.

Embodiment 2. The method of Embodiment 1, wherein the DNA substrate comprises genomic DNA.

Embodiment 3. The method of Embodiment 1, wherein the DNA substrate comprises DNA that has been pre-treated to protect 5hmC.

Embodiment 4. The method of Embodiment 3, wherein the DNA substate comprises DNA that has been pre-treated with an enzyme selected from DNA beta-glucotransferase (BGT), carbamoyl transferase, and a combination thereof.

Embodiment 5. The method of Embodiment 1, wherein the DNA substrate does not contain DNA that has been pre-treated with a TET methylcytosine dioxygenase.

Embodiment 6. The method of Embodiment 1, wherein prior to step (b) the DNA substrate is contacted with one or more other deaminating agents having a different specificity from the methyl cytosine-selective deaminase of (a).

Embodiment 7. The method of Embodiment 6, wherein the deaminating agent is a deaminase enzyme.

Embodiment 8. The method of Embodiment 1, wherein the DNA substrate further comprises an adaptor.

Embodiment 9. The method of Embodiment 8, wherein the adaptor further comprises a primer.

Embodiment 10. The method of Embodiment 1, wherein the methyl cytosine-selective deaminase preferentially deaminates methyl cytosine (mC) relative to cytosine (C).

Embodiment 11. The method of Embodiment 10, wherein the methyl cytosine is 5-methyl cytosine (5mC).

Embodiment 12. The method of Embodiment 1, wherein the methyl cytosine-selective deaminase is a phage deaminase or non-natural modification thereof.

Embodiment 13. The method of Embodiment 1, wherein the methyl cytosine-selective deaminase has an amino acid sequence that is at least 90% identical to any of SEQ ID NO: 1, 2, 3, 4, 5, 6, 7, 8, 9, 10, and 11.

Embodiment 14. The method of Embodiment 1, wherein the methyl cytosine-selective deaminase is selected from any of SEQ ID NO: 1, 2, 3, 4, 5, 6, 7, 8, 9, 10, and 11.

Embodiment 15. The method of Embodiment 1, wherein the methyl cytosine-selective deaminase is at least 90% identical to SEQ ID NO:1.

Embodiment 16. The method of Embodiment 1, further comprising, before sequencing, contacting the deamination product with a substance that generates a single nucleotide gap at the location of a uracil residue.

Embodiment 17. The method of Embodiment 16, wherein the substance is USER enzyme.

Embodiment 18. The method of Embodiment 1, further comprising preparing the DNA substrate prior to (a) in a method comprising:
(a) ligating a hairpin adaptor to a double-stranded genomic DNA sample to produce a ligation product;
(c) enzymatically generating a free 3′ end in a double-stranded region of the hairpin adaptor in the ligation product;
(d) extending the free 3′ end in a reaction mix that comprises a strand-displacing or nick-translating polymerase, dGTP, dATP, dTTP and modified dCTP, wherein the modified dCTP is deamination resistant to produce and extended product;
(e) to produce a single-stranded DNA substrate.

Embodiment 19. A kit comprising:
(a) a methyl cytosine-selective deaminase capable of converting 5-methylcytosine (5mC) to thymidine (T);
(b) one or more items selected from primer; polymerase; protecting agent for selected nucleobase or modified nucleobase (e.g., beta-glucosyltransferase); additional deaminase or deaminating treatment; reference DNA, including methylation reference; reference sequence; reaction buffer.

Embodiment 20. A method for base editing comprising:
contacting a fusion protein with a target sequence to produce an edited target sequence comprising at least one deaminated modified cytosine, wherein the fusion protein comprises a methylation-selective deaminase fused to a DNA binding domain.

Embodiment 21. The method of Embodiment 20, wherein the DNA binding domain is selected from a Cas9 domain, a Cas12 domain, a transcription activator-like effector nuclease (TALEN domain), a zinc finger (ZF) domain, a transcription activator-like effector (TALE) domain, and a methyl binding domain (MBD) domain.

Embodiment 22. The method of Embodiment 21, wherein the fusion protein further comprises a guide RNA complementary to at least a portion of the targeted sequence.

Embodiment 23. The method of Embodiment 22 wherein the fusion protein comprises an enzyme at is at least 90% identical to any of SEQ ID NOS:1-11.

Embodiment 24. A method, comprising:
contacting a DNA substrate comprising at least one modified cytosine (C) with a methyl cytosine-selective deaminase to produce a deamination product, wherein the methyl cytosine-selective deaminase is capable of converting 5-methylcytosine (5mC) to thymidine (T).

Embodiment 25. A fusion protein comprising an enzyme at is at least 90% identical to any of SEQ ID NOS: 1-11.

Embodiment 25. A sequencing method, comprising:
(a) contacting a DNA substrate comprising cytosine (C) and at least one methylcytosine nucleobase selected from 5-methylcytosine (5mC) and 5-hydroxymethylcytosine (5hmC) or comprising C and both 5mC and 5hmC, with a methylcytosine-selective deaminase to produce a deamination product, wherein the methylcytosine-selective deaminase (i) is capable of deaminating 5mC to thymidine (T) and/or 5hmC to hydroxymethyluridine (hmU) and (ii) preferentially deaminates the at least one methylcytosine nucleobase relative to cytosine (C); and
(b) sequencing the deamination product, or amplifying the deamination product to produce an amplification product, and sequencing the amplification product, in each case, to produce sequence reads,
wherein positions of Cs and position of the at least one methylcytosine nucleobase in the DNA substrate is determined based on the sequence reads.

Embodiment 26. The method of Embodiment 25, wherein the DNA substrate does not comprise DNA that has been pretreated to protect any nucleobase from deamination.

Embodiment 27. The method of Embodiment 26, wherein the DNA substrate does not comprise DNA that has been pretreated to protect 5mC from deamination.

Embodiment 28. The method of any preceding Embodiment, wherein the DNA substrate does not comprise DNA that has been pretreated with a TET methylcytosine dioxygenase.

Embodiment 29. The method of Embodiment 25, wherein the DNA substrate does not comprise DNA that has been pretreated to protect C from deamination.

Embodiment 30. The method of Embodiment 25, wherein the methylcytosine-selective deaminase preferentially deaminates 5mC relative to 5hmC and C, and the position of at least one 5mC in the DNA substrate is determined.

Embodiment 31. The method of Embodiment 25, wherein the DNA substrate comprises DNA that has been pre-treated to protect one or more methylcytosine nucleobases from deamination.

Embodiment 32. The method of Embodiment 31, wherein the pretreatment comprises incubation with an enzyme.

Embodiment 33. The method of Embodiment 32, wherein the enzyme protects 5hmC from deamination.

Embodiment 34. The method of Embodiment 33, wherein the positions of at least one 5mC, at least one 5hmC, and C are determined.

Embodiment 35. The method of Embodiment 34, wherein the enzyme is selected from beta-glucosyltransferase (BGT), carbamoyl transferase, glycotransferase selective for 5hmC, and a combination thereof.

Embodiment 36. The method of Embodiment 35, wherein the enzyme is BGT and the methylcytosine-selective deaminase does not deaminate glucosylated hmC.

Embodiment 37. The method of Embodiment 32, wherein the enzyme protects C from unwanted background deamination by the methylcytosine-selective deaminase.

Embodiment 38. The method of Embodiment 37, wherein the enzyme is selected from 4mC methylase and N4 methyltransferase.

Embodiment 39. The method of Embodiment 32, wherein the enzyme protects 5mC from deamination.

Embodiment 40. The method of Embodiment 39, wherein the enzyme is TET methylcytosine dioxygenase and the methylcytosine-selective deaminase does not deaminate 5-formylC (5fC) or 5-carboxyC (5caC).

Embodiment 41. The method of Embodiment 31, wherein the pretreatment comprises incubation with a chemical reagent.

Embodiment 42. The method of Embodiment 41, wherein the chemical reagent is pyridine borate.

Embodiment 43. The method of any preceding Embodiment, wherein the methylcytosine-selective deaminase of (a) is a mixture of two or more methylcytosine-selective deaminases.

Embodiment 44. The method of any preceding Embodiment, wherein the methylcytosine-selective deaminase of (a) does not deaminate C above background level.

Embodiment 45. The method of any preceding Embodiment, wherein the methylcytosine-selective deaminase is selective for a methylcytosine nucleobase present within a defined sequence context, wherein the methylcytosine-selective deaminase does not deaminate the methylcytosine nucleobase lacking the defined sequence context above background level.

Embodiment 46. The method of any preceding Embodiment, wherein the methylcytosine-selective deaminase comprises more than one methylcytosine-selective deaminase, each selective for 5mC and/or 5hmC present within different sequence contexts, wherein the methylcytosine selective deaminases do not deaminate the 5mC and/or 5hmC lacking the sequence context above background level.

Embodiment 47. The method of Embodiment 25, wherein prior to (b) the DNA substrate is contacted with one or more deaminating agents having a different specificity from the methylcytosine-selective deaminase of (a).

Embodiment 48. The method of Embodiment 47, wherein the deaminating agent is selected from an enzyme, a chemical reagent, and both.

Embodiment 49. The method any preceding Embodiment, wherein the deamination product is not amplified.

Embodiment 50. The method of Embodiment 49, wherein the deamination product is added directly to a sequencing machine.

Embodiment 51. The method of any preceding Embodiment, wherein the deamination product is amplified to produce an amplification product.

Embodiment 52. The method of Embodiment 51, wherein amplification comprises polymerase chain reaction, and a polymerase is optionally selected from Q5U polymerase, and a polymerase having a uracil-binding pocket.

Embodiment 53. The method of Embodiment 52, wherein the polymerase having a uracil-binding pocket is Q5.

Embodiment 54. method of Embodiment 25, wherein before (b), the deamination product is contacted with a substance that generates a single nucleotide gap at the location of a uracil (U).

Embodiment 55. The method of Embodiment 54, wherein the substance is selected from USER enzyme.

Embodiment 56. The method of Embodiment 25, wherein before (b), the deamination product is contacted with a substance that generates an abasic site at the location of a U and/or an 5hmU.

Embodiment 57. The method of Embodiment 56, wherein the substance is selected from uracil DNA glycosylase, single-strand-selective monofunctional uracil DNA glycosylase 1, thymine-DNA glycosylase and methyl-CpG binding domain protein 4.

Embodiment 58. The method of any preceding Embodiment, wherein the DNA substrate further comprises an adaptor.

Embodiment 59. The method of Embodiment 58 wherein the adaptor further comprises a sequencing primer binding site.

Embodiment 60. The method of Embodiment 58 or 59, wherein the adaptor further comprises a barcode and/or unique molecular identifier (UMI).

Embodiment 61. The method of any preceding Embodiment, wherein the DNA substrate comprises DNA selected from genomic DNA, ancient DNA, forensic DNA, environmental DNA, human DNA, free circulating DNA, synthetic DNA, plant DNA, and tumor DNA.

Embodiment 62. The method of any preceding Embodiment, wherein the DNA substrate further comprises RNA.

Embodiment 63. The method of any preceding Embodiment, wherein the methylcytosine-selective deaminase is a viral deaminase or non-natural modification thereof.

Embodiment 64. The method of Embodiment 63, wherein the viral deaminase is a bacteriophage-encoded deaminase or modification thereof.

Embodiment 65. The method of any preceding Embodiment, wherein the methylcytosine-selective deaminase comprises a B5 deaminase signature sequence.

Embodiment 66. The method of Embodiment 25, wherein the methylcytosine-selective deaminase comprises an amino acid sequence that is at least 90% identical to any of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55.

Embodiment 67. The method of Embodiment 66, wherein the methylcytosine-selective deaminase comprises an amino acid sequence selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54, and 55.

Embodiment 68. The method of Embodiment, wherein the methylcytosine-selective deaminase comprises an amino acid sequence that is at least 90% identical to any of SEQ ID NOS:1, 5 and 14.

Embodiment 69. The method of Embodiment 25, further comprising preparing the DNA substrate prior to (a) in a method comprising:
(a) ligating a hairpin adaptor to a double-stranded genomic DNA sample to produce a ligation product;
(b) enzymatically generating a free 3′ end in a double-stranded region of the hairpin adaptor in the ligation product;
(c) extending the free 3′ end in a reaction mix that comprises a strand-displacing or nick-translating polymerase, dGTP, dATP, dTTP and modified dCTP, wherein the modified dCTP is deamination resistant to produce and extended product;
to thereby produce a single-stranded DNA substrate.

Embodiment 70. A method for selective deamination of a DNA substrate, comprising:
contacting a DNA substrate comprising cytosine (C) and at least one methylcytosine nucleobase selected from 5-methylcytosine and 5-hydroxymethylcytosine, with a methylcytosine-selective deaminase, to produce a deamination product, wherein the methylcytosine-selective deaminase (i) is capable of deaminating 5mC to thymidine (T) and/or 5hmC to hydroxymethyluridine (hmU), (ii) preferentially deaminates 5mC and/or 5hmC relative to cytosine (C), and (iii) and is characterized by having at least one feature selected from: (a) the methylcytosine-selective deaminase comprises a B5 deaminase signature sequence; and (b) the methylcytosine-selective deaminase is encoded by a viral gene located in a viral genome in proximity to a thymidylate synthase gene, and (c) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55.

Embodiment 71. A method for nucleobase editing, comprising: contacting a fusion protein comprising a methylation-selective deaminase capable of deaminating 5mC to thymidine (T) fused to a DNA binding domain, with a target sequence comprising cytosine (C) and one or more 5-methylcytosine (5mC), to produce an edited target sequence comprising at least one T generated by deaminating 5mC, wherein the methylation-selective deaminase preferentially deaminates 5mC relative to C, and is characterized by having at least one of the features selected from: (i) the methylcytosine-selective deaminase comprises B5 deaminase signature sequence and (ii) the methylcytosine-selective deaminase is encoded by a viral gene located in a viral genome in proximity to a thymidylate synthase gene, and (iii) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55.

Embodiment 72. The method of Embodiment 71, wherein the methylcytosine-selective deaminase is selective for 5mC present within a sequence context, wherein the methylcytosine-selective deaminase does not deaminate 5mC lacking the sequence context above background level.

Embodiment 73. The method of Embodiment 71 or 72 wherein the DNA binding domain is selected from a Cas9 domain, a Cas12 domain, a transcription activator-like effector nuclease (TALEN domain), a zinc finger (ZF) domain, a transcription activator-like effector (TALE) domain, and a methyl binding domain (MBD) domain.

Embodiment 74. A kit for methylation sequencing, comprising:
(a) a methylcytosine-selective deaminase that (i) is capable of deaminating 5-methyl cytosine (5mC) to thymidine (T) and/or 5-hydroxylmethyl cytosine (5hmC) to hydroxymethyluridine (hmU) and (ii) preferentially deaminates 5mC and/or 5hmC relative to cytosine (C); and
(b) one or more items selected from a primer; adaptor, polymerase; reverse transcriptase; ligase; protecting agent for selected nucleobase and/or modified nucleobase; another deaminase and/or deaminating treatment; reference DNA, methylated reference DNA; reference sequence; reaction buffer.

Embodiment 75. The kit of Embodiment 74, wherein the methylcytosine-selective deaminase is characterized by having at least one of the features selected from (i) the methylcytosine-selective deaminase comprises a B5 deaminase signature sequence; and (ii) the methylcytosine-selective deaminase is encoded by a viral gene located in a viral genome in proximity to a thymidylate synthase gene, and (iii) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 53, 54 and 55.

Embodiment 76. A methylcytosine-selective deaminase, comprising:
a polypeptide that is capable of deaminating 5-methyl cytosine (5mC) to thymidine (T) and/or 5-hydroxylmethyl cytosine (5hmC) to hydroxymethyluridine (hmU); and preferentially deaminates 5mC and/or 5hmC relative to cytosine (C);
wherein the polypeptide is selected from:
   (i) a non-natural fusion protein comprising a methylcytosine-deaminase domain comprising a B5 deaminase signature sequence;
   (ii) a non-natural fusion protein comprising a methylcytosine-selective deaminase domain encoded by a viral gene, wherein the viral gene is located in a viral genome in proximity to a thymidylate synthase gene;
   (iii) a non-natural fusion protein comprising an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 53 and 54; and
   (iv) a polypeptide comprising an amino acid sequence selected from SEQ ID NOS: 37, 38, 39, and 40.

Embodiment 77. The deaminase of Embodiment 76, wherein the polypeptide is a non-natural fusion protein comprising a methylcytosine-selective signature sequence, and the methylcytosine-selective deaminase portion of the fusion protein comprises is encoded by a viral gene.

Embodiment 78. A composition, comprising a methylcytosine deaminase of Embodiment 76 or 77 and a component selected from a solid support, a buffer, and a reaction mixture.

Embodiment 79. A kit for deaminating a DNA substrate, comprising a deaminase of any prior Embodiment and one or more items selected from a reaction buffer, primer; adaptor, polymerase; reverse transcriptase; ligase; protecting agent for selected nucleobase and/or modified nucleobase; another deaminase and/or deaminating treatment; reference DNA, methylated reference DNA; reference sequence; reaction buffer.

### EXAMPLES

### Example 1. Identification of Phage Deaminases that Deaminate 5hmC in an E. coli Expression System

This example describes hydroxymethylcytosine specific deaminating activity of phage deaminases using an in vivo system.

Methylcytosine-selective deaminases B5, C4, D5, E8, E10, F4, and H10 were recombinantly expressed from pET28 ((Novagen, Darmstadt, Germany) in E. coli C2566 simultaneously carrying a pETDuet expression plasmid (Novagen) encoding both a dCMP hydroxymethyltransferase from bacteriophage T4 (gp42) and a promiscuous dNMP kinase (gp1). Expression of T4 gp42 and gp1 together leads to the production of 5-hydroxymethyl-2′-deoxycytosine triphosphate in vivo, where this non-canonical nucleotide is incorporated into genomic and plasmid DNA by E. coli DNA polymerase. The non-canonical nucleoside hm5-dC (also referenced as 5hmdC) was chosen because its deamination product hm5-dU (also referenced as 5hmdU) is also a non-canonical nucleoside allowing this product to be distinguished from the other endogenous bases in DNA. Co-expression of the methylcytosine-selective deaminases described herein with T4 gp42 and gp1 results in conversion of hm5C to hm5U in genomic and plasmid DNA.

For these experiments, each methylcytosine-selective deaminase was expressed simultaneously with hydroxymethyltransferase and kinase for 12 hours; plasmid DNA was then recovered from the cultures that had expressed. The nucleotide composition of this plasmid DNA was analyzed as previously described (Lee Y J, Weigele P R. Detection of Modified Bases in Bacteriophage Genomic DNA. Methods Mol Biol. 2021; 2198:53-66. doi: 10.1007/978-1-0716-0876-0_5. PMID: 32822022.). Briefly plasmid DNA was enzymatically digested to free nucleosides using NEB Nucleoside digestion mix (NEB #M0649, New England Biolabs, Ipswich, MA) and resolved via C18 reverse phase UPLC eluted by buffered gradient of methanol. Nucleosides were detected as they eluted from the reverse-phase column by UV/Absorbance producing a chromatogram of absorbance peaks eluting at various time points during the method.

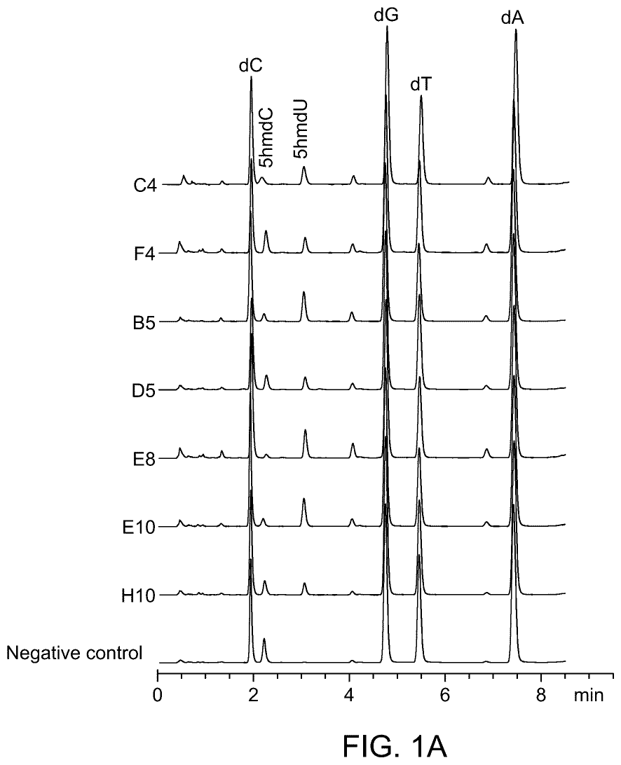

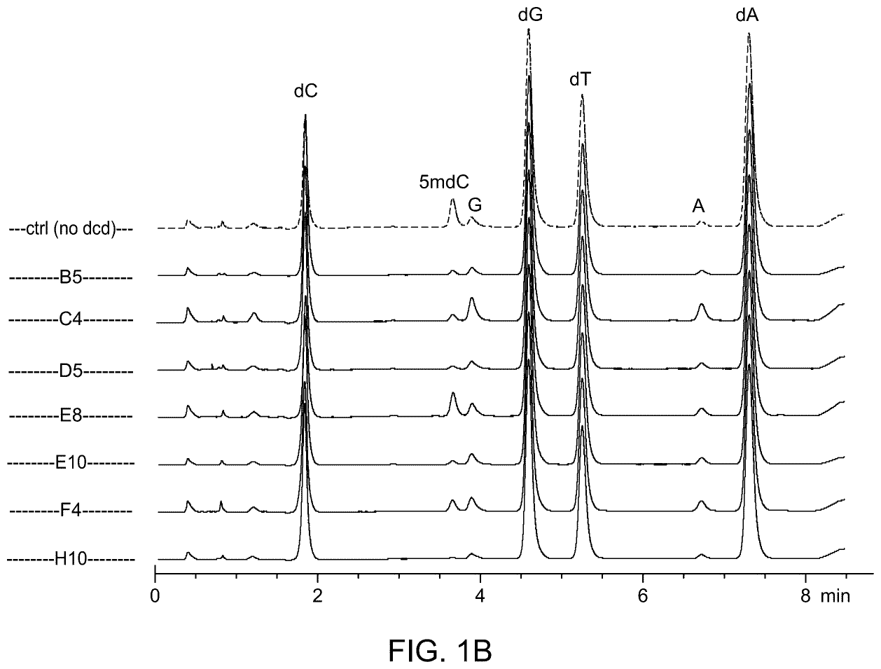

As seen in FIG. 1A, DNA recovered from E. coli cells expressing T4 gp42 and gp1 alone contains a mixture of the nucleosides dA, dG, dC and dT, as well as the non-nucleoside 5-hydroxymethyl-2′deoxycytosine (5hmdC). Cells expressing methylcytosine-selective deaminases (B5, C4, D5, E8, E10, F4, and H10) were found to also contain 5-hmdU (also referenced as 5hmdU), but not dU, indicating a preference for deaminating cytosine with a hydroxymethyl substituent at C5 as opposed to unmodified cytosine. As seen in FIG. 1B, the 5mC peaks are smaller compared to the control experiment indicating that the 5mdC is deaminated. Thus, FIG. 1A-1B shows that methylcytosine-selective deaminases B5, C4, D5, E8, E10, F4, and H10 are capable of deaminating 5hmC and 5mC in genomic and plasmid DNA, and preferentially deaminate 5hmC and 5mC over C.

### Example 2. Deamination Activity Toward Modified Cytosine in Oligonucleotide Substrates

This example describes methylcytosine deaminating activity on DNA oligonucleotides containing 5hmC by B5 deaminase using an in vitro system.

The B5 deaminase sequence was synthesized by assembly of oligo pools and cloned into pET28 expression vector encoding an N-terminal hexa-histidine tag to facilitate purification by immobilized metal affinity chromatography. The deaminase was expressed in E. coli C2566 and purified from cellular lysates using a nickel column (GE Life Sciences) on an FPLC system.

The B5 deaminase was assayed using DNA substrates containing either modified and/or unmodified cytosines. Substrates used were bacteriophage genomic DNAs extracted from E. coli phage T4gt (a mutant phage T4 strain lacking DNA glucosyltransferase activity) which contains hm5-dC fully substituting for dC and Escherichia phage lambda (I), which contains canonical nucleobases only.

Conditions for activity against DNA containing C fully substituted with 5hmC: 40 μL reactions containing 500 ng of heat-denatured, genomic DNA substrate (^{˜}4.5-10 fmol total cytosines) containing deaminase or an APOBEC control at 6 μM in 1× Dpnll buffer were incubated at 30° C. for 12 hours, and then enzymatically hydrolyzed to free nucleosides, resolved by HPLC, and visualized by UV-absorbance as described above. Concentrated (i. e. 10×) Dpnll buffer contains 50 mM Bis-Tris-HCl, 100 mM NaCl, 10 mM MgCl2, and 0.1 mM DTT at pH 6.

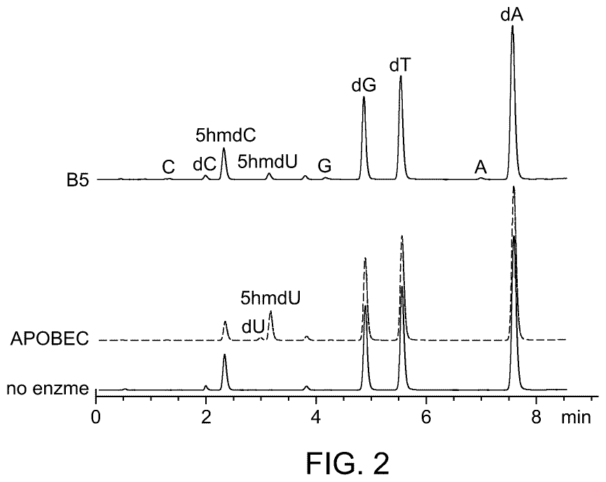

As seen in FIG. 2, deamination of T4gt DNA 5hmC produces 5hmU. Under these experimental conditions, the B5 deaminase was able to deaminate a portion of the 5hmC present in the reaction.

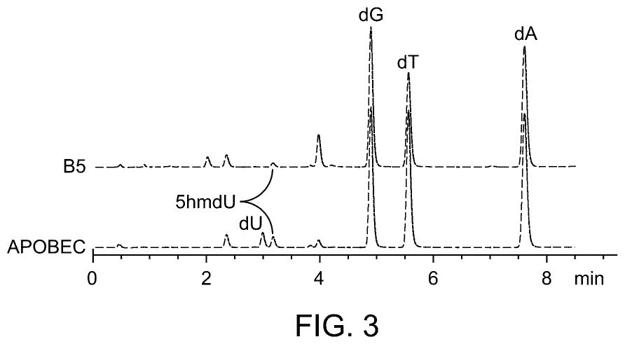

B5 deaminase was tested for deamination of C, 5mC and 5hmC using DNA polymer substrates containing C, 5mC, and 5hmC (lambda, XP12, and T4gt), with each of these substrates present in the same reaction (again, 500 ng total). As seen in FIG. 3, B5 deaminase in BIS-TRIS pH 6.5 was able to convert 5hmC to 5hmU (seen as a new peak) and 5mC to T (seen as a decrease in the 5mC peak relative to the no-enzyme control (no-enzyme control not shown in FIG. 3)). Notably, and in contrast to reactions containing APOBEC, no dU was formed by B5 deaminase in this experiment, indicating a greater activity of the B5 deaminase towards modified cytosines versus C.

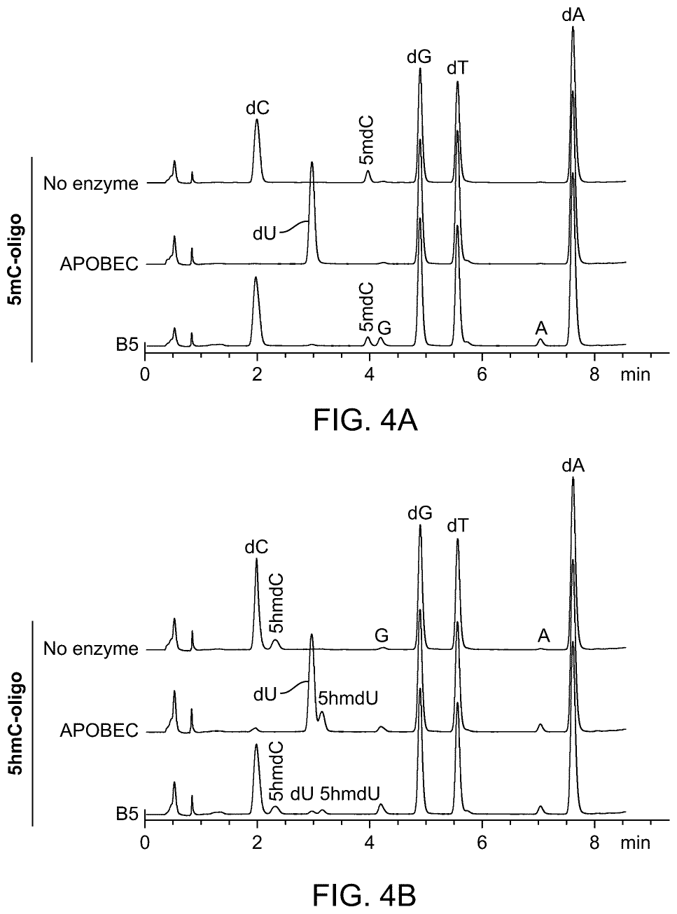

B5 deaminase was also tested using ssDNA 20-mer oligonucleotide substrates containing either a single 5mC or a single 5hmC together with dC using an LC/MS assay. The oligo sequences were 5′-TGTCCGATAGACT(5mC)TACGCA-3′ (SEQ ID NO:41) (“5mC-oligo” in FIG. 4A; Oligo 2 in Table 1)) and 5′-TGTCCGATAGACT(5hmC)TACGCA-3′ (SEQ ID NO:42:) (“5hmC-oligo” in FIG. 4B; Oligo 1 in Table 1).

Reactions of 40 μL total volume and containing 6 μM B5 deaminase, ^{˜}3.2 uM oligo substrate in a 1×Dpnll buffer were incubated at 30° C. for 12 hours. Following incubation, the reactions were enzymatically hydrolyzed to free nucleosides and resolved/visualized as described above. As seen in FIG. 4A-4B, only a small amount of C was converted to dU (as compared to APOBEC), but relatively more 5mC and 5hmC were converted to T and 5hmdU, respectively.

TABLE 1
Digestion of Oligonucleotide Substrates

| | | | | | | | % hmC-> | % mC-> |
|---|---|---|---|---|---|---|---|---|
| Substrate/Enzyme | dC | dU | d5mC | D5hmC | D5hmU | % C->U | hmU | T |
| Oligo 1 only | 1.02 | 0 | 0.23 | | 1.05 | 0% | 0% | |
| Oligo 1 + B5 | 1.06 | 0.01 | 0.14 | | 1.17 | 0.90% | 35.00% | |
| Oligo 1 + APOBEC | 0 | 0.97 | 0 | | 1.29 | 100.00% | 100% | |
| Oligo 2 only | 1.01 | 0 | | 0.17 | 0 | 0% | | 0% |
| Oligo 2 + B5 | 1.04 | 0.03 | | 0.15 | 0.04 | 2.80% | | 21.10% |
| Oligo 2 + APOBEC | 0.06 | 0.95 | | 0 | 0.21 | 94.10% | | 100% |

### Example 3. Selective Deamination of Modified Cytosine Nucleotides by B5 Deaminase

This example describes activity of B5 deaminase on 2′-deoxynucleotidetriphosphate dCTP as well as non-canonical nucleotides 5hmdCTP and 5mdCTP. Activity of B5 deaminase was tested in 20 μL reactions containing 0.25 mM nucleotides, about 8 μM enzyme, 0.25 mM dGTP (as an internal concentration standard) and 50 mM BIS-TRIS pH 6.5 at 42° C. for 30 minutes. The reactions were subsequently treated with recombinant calf intestinal phosphatase (NEB #M0525) for one hour to convert the nucleotides followed by heat-inactivation of enzymes at 80° C. for 10 minutes. Free nucleoside mixtures containing substrates, products, and the internal concentration standard were resolved by reverse-phase liquid chromatography and detected by UV absorption as described above.

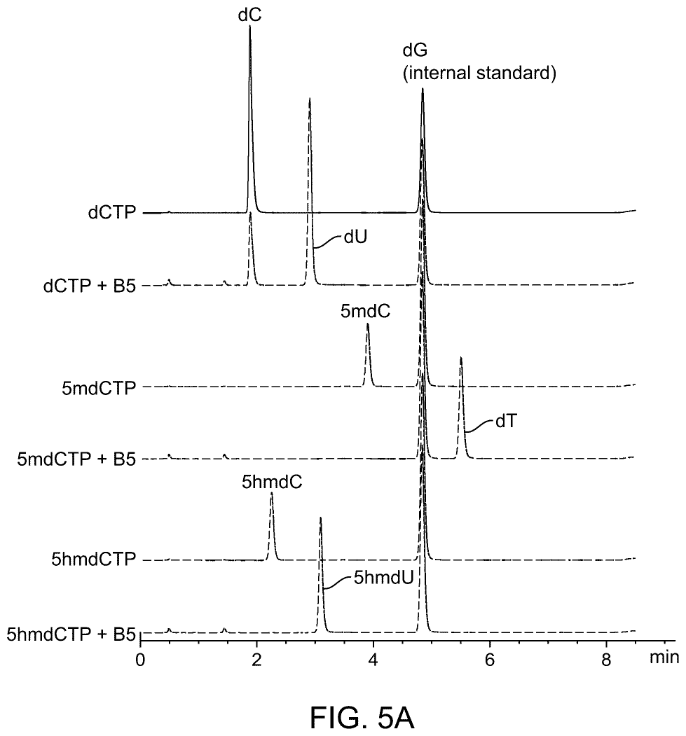

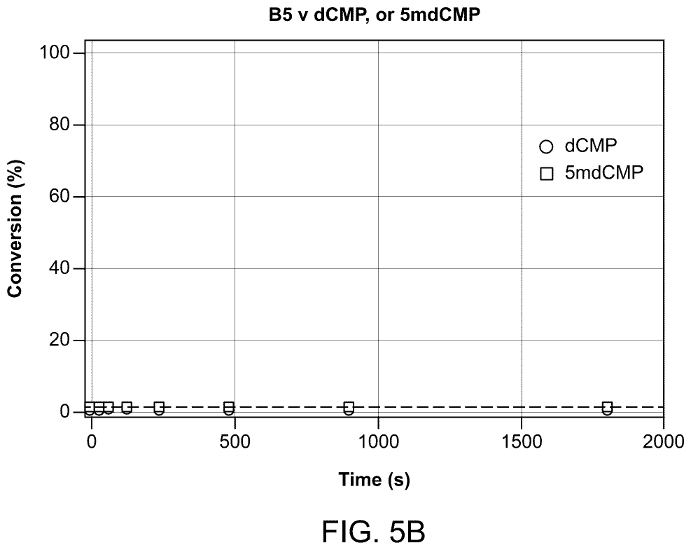

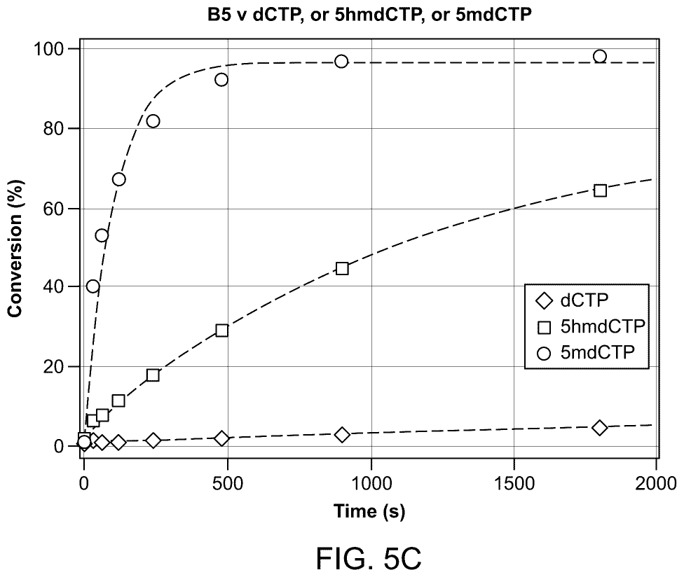

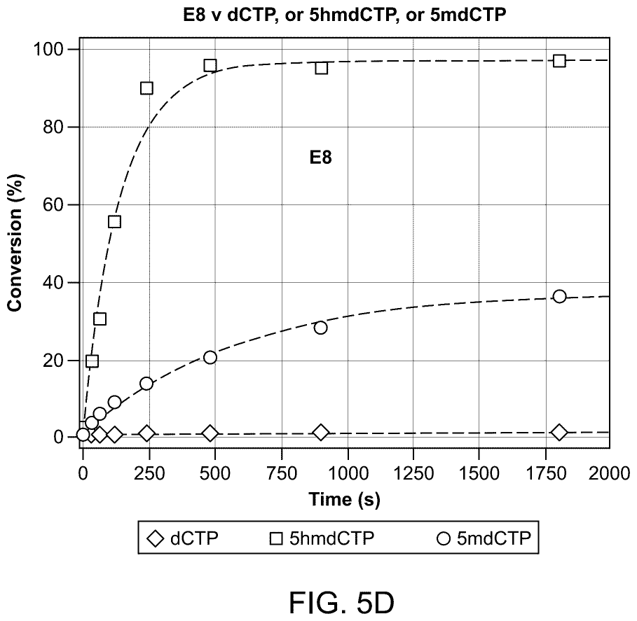

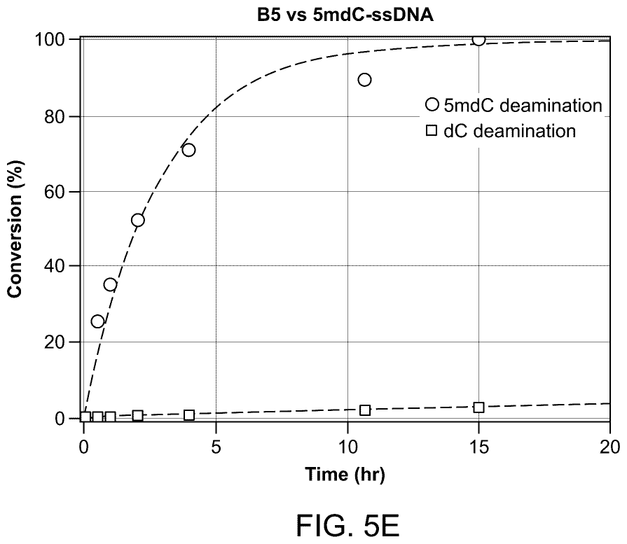

As seen in FIG. 5A, B5 deaminase completely converted 5mdCTP and 5hmdCTP to their deaminated forms, whereas deamination of dCTP was incomplete, indicating a preference of this deaminase for the modified nucleobases. In these experiments, substrate is in 30-fold excess over enzyme. Furthermore, it should be noted that the activity of B5 deaminase towards triphosphate nucleotides is unusual compared to the larger family of mononucleotide deaminases to which it belongs (aka dCMP deaminase). B5 also efficiently deaminated 5hmdCTP, albeit at a slower rate than 5mdCTP (FIG. 5C). Additionally, the enzyme showed preference for nucleotide triphosphate versus monophosphates. Very little or no activity was observed on either dCMP or 5mdCMP. The ability to deaminate modified nucleotides in the context of a DNA polymer may be related to this enzyme's ability to deaminate triphosphate forms of 5mdC and 5hmdC. FIG. 5B shows a time course of B5 deamination of dCMP and 5mdCMP; FIG. 5C shows a time course of B5 deamination of dCTP, 5hmdCTP and 5mdCTP. FIG. 5D shows a time course of E8 deamination of dCTP, 5hmdCTP and 5mdCTP. FIG. 5E shows a time course of B5 deamination of a single 5mC in a single-stranded oligo.

### Example 4. Ribonucleotide Deamination

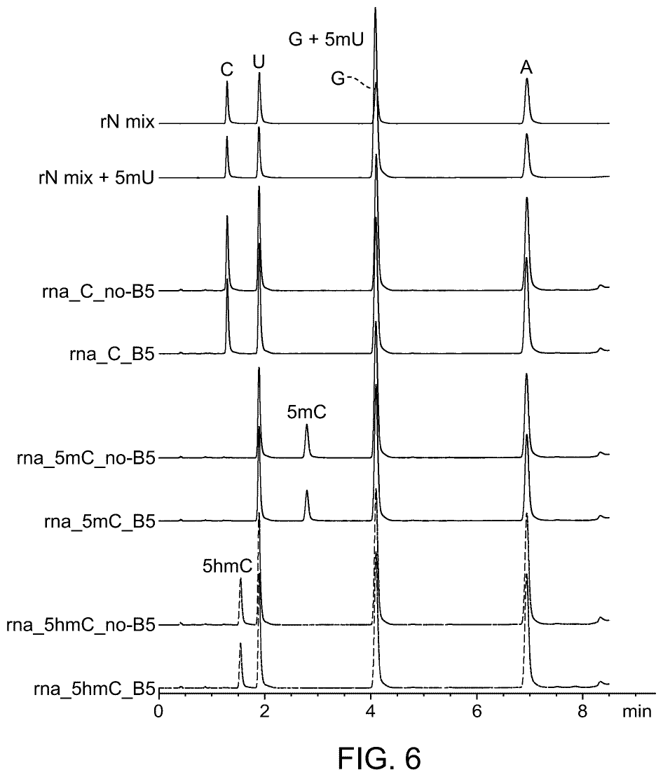

Activity of deaminase B5 on ribonucleotides was tested in 20 μL reactions containing 0.25 mM nucleotides, ^{˜}8 μM enzyme, 0.25 mM dGTP (as an internal concentration standard, and 50 mM BIS-TRIS pH 6.5 at 42° C. for 30 minutes. The reactions were subsequently treated with recombinant calf intestinal phosphatase (NEB #M0525) for one hour to convert the nucleotides followed by heat-inactivation of enzymes at 80° C. for 10 minutes. Free nucleoside mixtures containing substrates, products, and the internal concentration standard were resolved by reverse-phase liquid chromatography and detected by UV absorption as described above. FIG. 6 shows deamination of C (rna_C); 5mC (rna_5mC); 5hmC (rna_5mhC).

### Example 5. Selective Deamination of 5mC by Methylcytosine-Selective Deaminases in Oligonucleotides

This example describes the capability of four methylcytosine-selective deaminases disclosed herein to selectively deaminate 5mC, using a EcoRI cleavage as a readout in a capillary electrophoresis (CE) method.

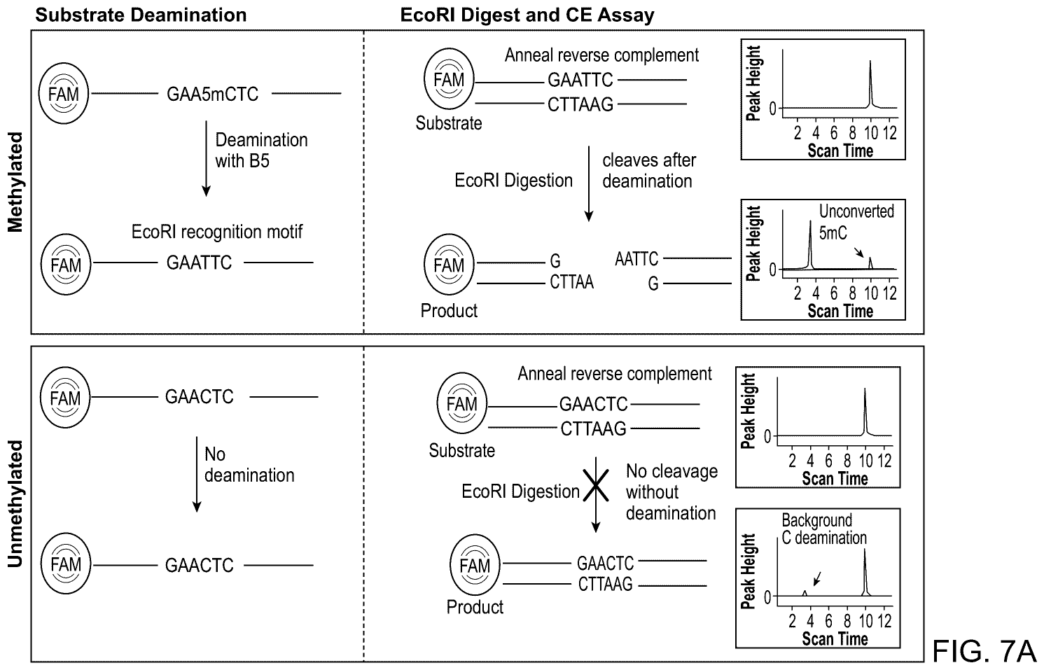

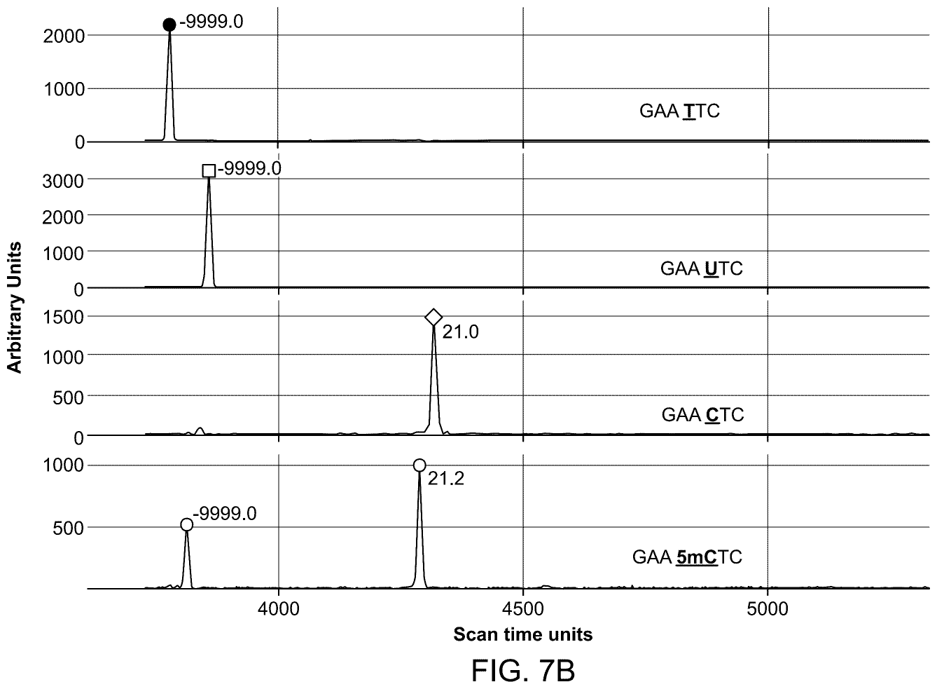

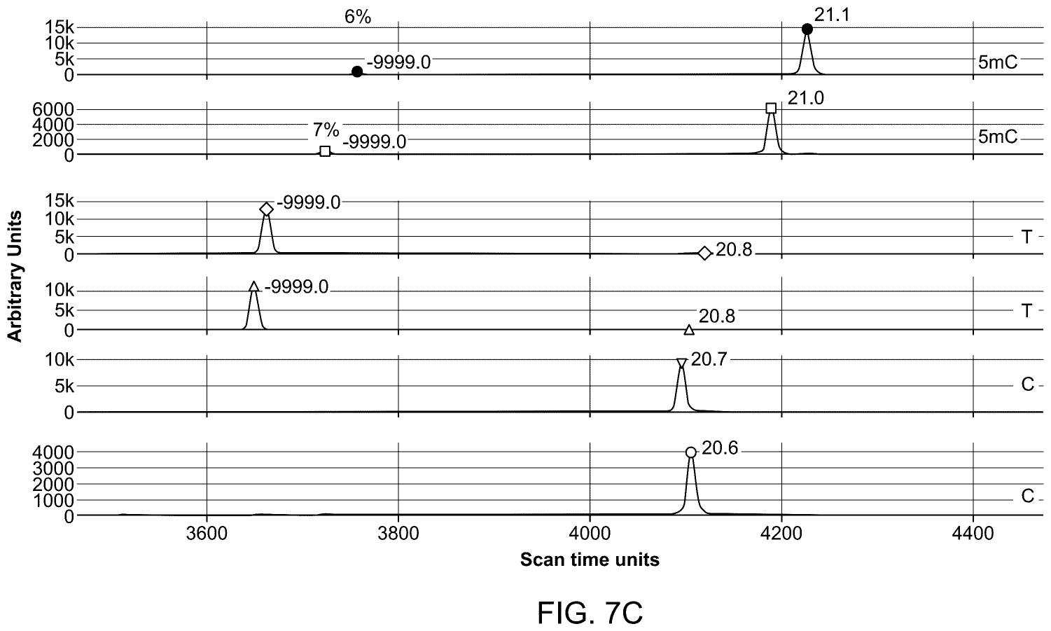

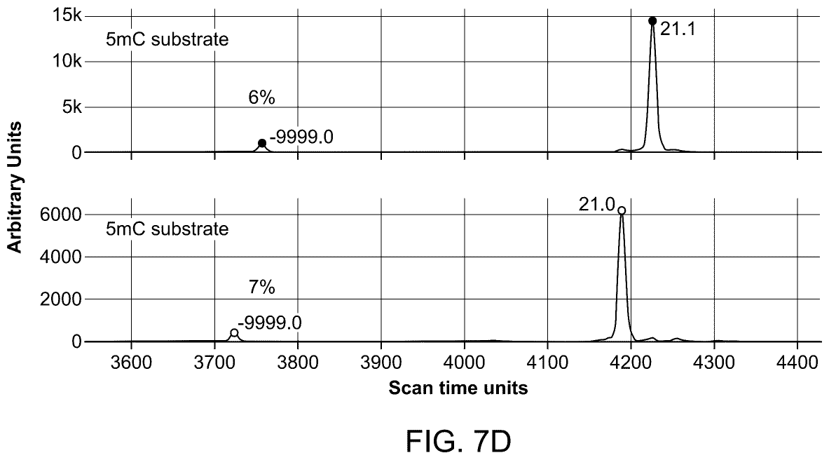

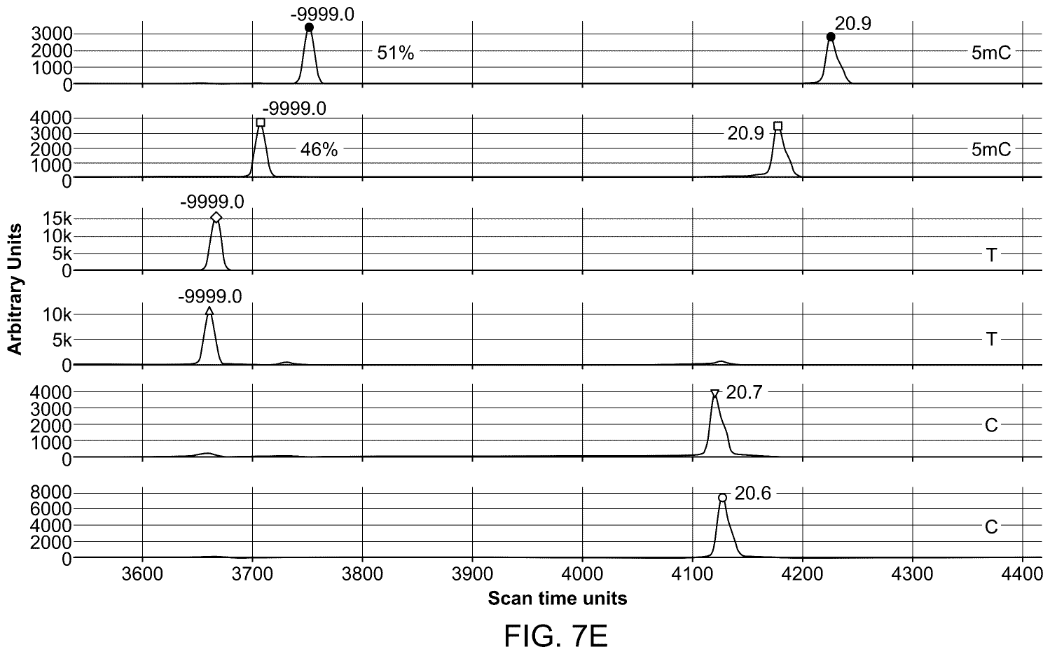

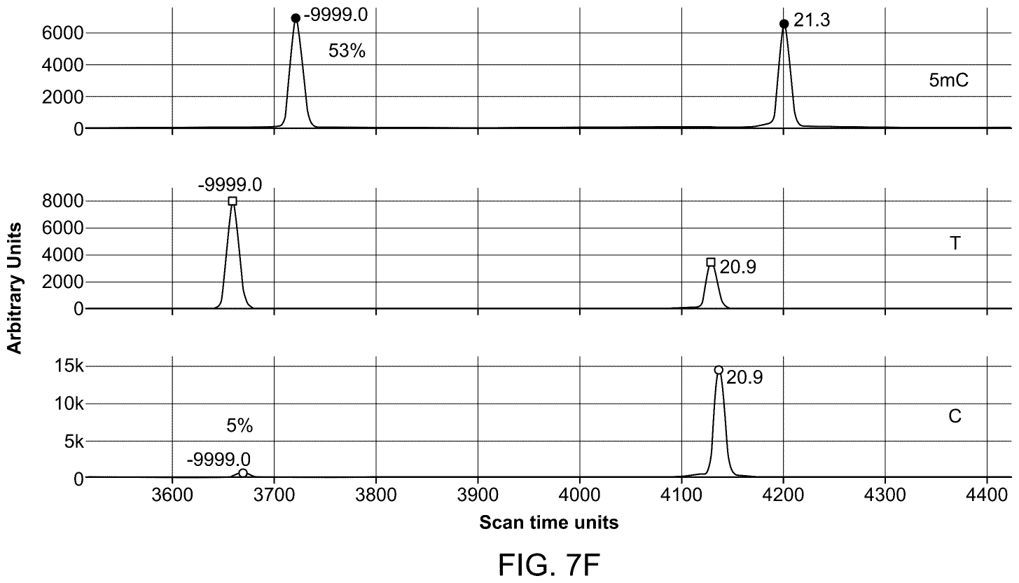

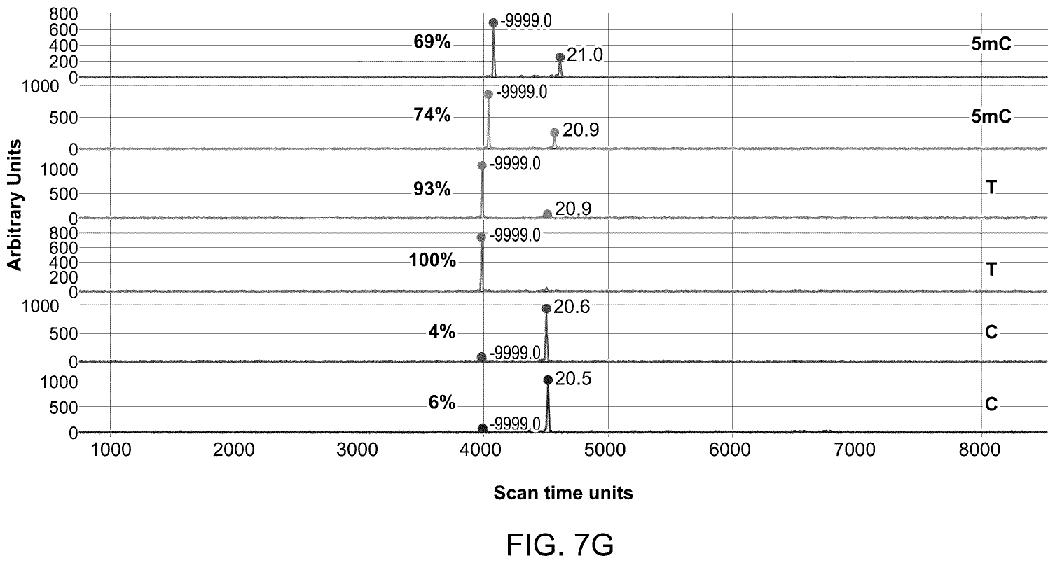

As depicted in FIG. 7A, oligonucleotides labeled with a fluorescent tag (FAM) were synthesized to either contain an EcoRI cleavage site (GAATTC or GAA(dU)TC) or non-EcoRI cleavable site containing 5mC or C, which could be restored as an EcoRI cleavage site if 5mC is converted to T by a deaminase or if C is converted to U by a deaminase (GAA(5mC)TC or GAA(C)TC)(see complete oligo sequences in Table 2 below).

The B5 deaminase selectively deaminated 5mC in this oligonucleotide context. The four oligonucleotides shown in Table 2 were added to the purified B5 deaminase (^{˜}7.5 μM B5 (lot1), 250 nM oligo (30:1), BIS-TRIS pH 6.5 buffer, 42° C. 2-3 hr). The reverse complement oligo (GCGTAAGCCCATGAATTCTGCATCGGTGTAT) (SEQ ID NO:43)(2-fold concentration) was subsequently annealed to the reaction (Heat to 95° C. and cool to room temperature In EcoRI buffer) and buffer exchanged to remove Zn ions before performing EcoRI digestion (condition following manufacturer protocol, 1H digestion 100 nM oligo concentration). The mixture was subsequently treated with Proteinase K and diluted to 10 nM for compatibility with the capillary electrophoresis system. The double-stranded oligos were treated with EcoRI (condition following manufacturer protocol, 1H digestion 100 nM oligo concentration). Digestion by EcoRI indicates that 5mC was converted to T by B5 deaminase. Control oligo 2 containing GAATTC was completely digested (FIG. 7B) demonstrating the expected complete activity of EcoRI enzyme on control oligos containing both T and dU. The digestion also indicated that B5 deaminase did not deaminate unmodified cytosine in GAATTC context. Oligos containing GAACTC showed no or insignificant digestion (FIG. 7B) showing no or little activity of B5 deaminase on C. Oligos containing GAA(5mC)TC show 36% digestion indicating a % deamination of 5mC by B5 deaminase of 36% under these experimental conditions. Peaks labelled −9999 correspond to fully cleaved oligonucleotides and peaks labelled 21.0/21.2 (retention time) correspond to uncleaved oligonucleotides. See also FIG. 7G, which shows that B5 selectively deaminated 5mC in oligonucleotide context. For the data generated in FIG. 7G, B5 was incubated in activity buffer containing 100 μM ZnCl2 for 2-3 hrs. The Zn was filtered using a Zymo plate filter (D4061).

The E10 deaminase also selectively deaminated 5mC in this oligonucleotide context. Oligonucleotides 1, 2 and 3 were added to the purified E10 deaminase, in duplicate (^{˜}7.5 μM E10 (lot0), 250 nM oligo (30:1), BIS-TRIS pH 6.5 buffer, 42° C., incubated overnight). The reverse complement oligo (GCGTAAGCCCATGAATTCTGCATCGGTGTAT) (SEQ ID NO:43) (2-fold concentration) was subsequently annealed to the reaction (heat to 95° C. and cool to room temperature In EcoRI buffer) before performing EcoRI digestion (condition following manufacturer protocol, 1H digestion 100 nM oligo concentration). The mixture was subsequently treated with Proteinase K and diluted to 1 nM for compatibility with the capillary electrophoresis system. As above, digestion by EcoRI indicates that 5mC was converted to T by the deaminase. Control oligo 2 containing GAATTC were 97% and 96% digested (FIGS. 7C and 7D) demonstrating the expected activity of EcoRI enzyme on control oligos containing T. The digestion also indicated that E10 deaminase did not deaminate unmodified cytosine in GAATTC context Oligonucleotides containing GAACTC show no or insignificant digestion (FIG. 7C) showing no or little activity of E10 deaminase on C. Oligos containing GAA(5mC)TC show 6-7% digestion indicating 6-7% deamination of 5mC by E10 deaminase under these experimental conditions.

Additionally, the Bursaria chlorella virus 1 (PBCV-1) PBCV-1 deaminase selectively deaminated 5mC in this oligonucleotide context. This enzyme is known to deaminate nucleotides; its capability in deaminating 5mC in oligonucleotide context was previously unrecognized (see, e.g., Zhang Y, Maley F, Maley G F, Duncan G, Dunigan D D, Van Etten J L. Chloroviruses encode a bifunctional dCMP-dCTP deaminase that produces two key intermediates in dTTP formation. J Virol. 2007 July; 81(14):7662-71. doi: 10.1128/JVI. 00186-07. Epub 2007 May 2. PMID: 17475641; PMCID: PMC1933376).

Three oligonucleotides shown in Table 2—the 5mC, T, and C substrates—were added to the purified PBCV-1 deaminase, in duplicate (^{˜}7.5 μM PBCV-1 (lot0), 250 nM oligo (30:1), BIS-TRIS pH 6.5 buffer, 42° C., incubated overnight). The reverse complement oligo (GCGTAAGCCCATGAATTCTGCATCGGTGTAT) (SEQ ID NO:43) (2-fold concentration) was subsequently annealed to the reaction (heat to 95° C. and cool to room temperature in EcoRI buffer) before performing EcoRI digestion (condition following manufacturer protocol, 1H digestion 100 nM oligo concentration). The mixture was subsequently treated with Proteinase K and diluted to 1 nM for compatibility with the capillary electrophoresis system. As above, digestion by EcoRI indicates that 5mC was converted to T by PBCV-1 deaminase. Control oligo 2 containing GAATTC were completely digested (FIG. 7E) demonstrating the expected activity of EcoRI enzyme on control oligos containing T. The digestion also indicated that PBCV-1 did not deaminate unmodified cytosine in GAATTC context. Oligos containing GAACTC show no or insignificant digestion (FIG. 7E) showing no or little activity of PBCV-1 CDA on C in the context of a polynucleotide. Oligos containing GAA(5mC)TC show 46-51% digestion indicating 46-51% deamination of 5mC by PBCV-1 (FIG. 7E).

Further, the C73 deaminase selectively deaminated 5mC in this oligonucleotide context. Three oligonucleotides shown in Table 2—the 5mC, T, and C substrates—were added to the purified C73 deaminase, in duplicate (^{˜}7.5 μM C73 (lot0), 250 nM oligo (30:1), BIS-TRIS pH 6.5 buffer, 42° C., incubated overnight). The reverse complement oligo (GCGTAAGCCCATGAATTCTGCATCGGTGTAT) (SEQ ID NO:43) (2-fold concentration) was subsequently annealed to the reaction (heat to 95° C. and cool to room temperature In EcoRI buffer) before performing EcoRI digestion (condition following manufacturer protocol, 1H digestion 100 nM oligo concentration). The mixture was subsequently treated with Proteinase K and diluted to 1 nM for compatibility with the capillary electrophoresis system. As above, digestion by EcoRI indicates that 5mC was converted to T by C73 deaminase. Control oligo 2 containing GAATTC were digested (FIG. 7F) demonstrating the expected activity of EcoRI enzyme on control oligos containing T. Oligos containing GAACTC show minimal digestion (FIG. 7F) demonstrating minimal activity of C73 deaminase on C when incubated overnight (5%). Oligos containing GAA(5mC)TC show 53% digestion indicating 53% deamination of 5mC by C73.

TABLE 2
Fam labelled oligonucleotides

| Oligo | Base | Sequence | SEQ ID NO: |
|---|---|---|---|
| 1 | 5mC | 56-FAM/ATA CAC CGA TGC AGA A/iMe-dC/T | 44 |
| | | CAT GGG CTT ACG C | |
| 2 | T | 56-FAM/ATA CAC CGA TGC AGA ATT CAT GGG | 45 |
| | | CTT ACG C | |
| 4 | C | 56-FAM/ATA CAC CGA TGC AGA ACT CAT GGG | 46 |
| | | CTT ACG C | |
| 5 | dU | 56-FAM/ATA CAC CGA TGC AGA A/ideoxyU/T CAT | 47 |
| | | GGG CTT ACG C | |

Assay results collected by CE for methylcytosine-selective deaminases are summarized in Table 3. The data shown include the highest measured activity of deaminases on 5mC and C from samples purified on a large scale from liters of expression culture on multiple columns (B5, E10, F7, H3), on a small scale from mLs of expression culture using IMAC resin only (PBCV-1), or immobilized on a solid support from <5 mL of expression culture.

TABLE 3
Summary of results of CE assay results for methylcytosine-
selective deaminases using oligo substrates

| | Assay time | | | |
|---|---|---|---|---|
| Mode | (hr) | Name | 5mC | C |
| In | 2 to 3 | B5 + Zn | 74 | 4 |
| solution | 2 to 3 | E10 | 54 | 3 |
| | Overnight | PBCV-1 | 51 | 7 |
| | 2 to 3 | F7 + Zn | 72 | 12 |
| | 2 to 3 | H3 | 60 | 2 |
| | Overnight | C73 | 53 | 5 |
| | 1 | PRW294 | 73 | <1 |
| | 1 | PRW311 | 69 | <1 |
| | 1 | PRW313 | 90 | 11 |
| | 1 | PRW314 | 90 | 13 |
| On solid | Overnight | F12 | 10 | ND |
| support | | H2 | 7 | ND |
| | | E12 | 5 | ND |
| | | B4 | 3 | ND |
| | | G10 | 4.5 | 2.6 |
| | | 206 | 50 | — |
| | | 251 | 66 | — |
| | | 252 | 12 | — |

ND is not detected;
— is not measured

### Example 6. Sequence Context of Methylcytosine-Selective Deamination by B5 Deaminase

This example describes methylcytosine-selective deaminase activity of B5 deaminase on E. coli genomic DNA using NGS sequencing to quantify the 5mC to T conversion at CCWGG sites (with C=5mC).

E. coli K12 (dcm⁺) genomic DNA was sheared using acoustic shearing and adaptors were ligated following the manufacturer recommendation (NEB, E7645S). E. coli K12 (dcm+) strain expresses the enzyme DNA-cytosine methyltransferase (dcm). This enzyme methylates the C-2 position of both strands of the sequence 5′-CCWGG-3′ (W=A or T). 5mC methylation in E. coli K12 (dcm+) is exclusively found in this context. Thus, DNA isolated from this strain contains methylcytosine (5mC) at C-2 of this sequence. For loop adaptors from a commercial library preparation kit (New England Biolabs), the ligation products were subsequently treated with USER (NEB, M5505S) following the manufacturer recommendation. 50 ng of DNA was then heat denatured (95° C.) and immediately put on ice. Deamination was performed using purified B5 deaminase at three incubation temperatures: 30°, 37° and 42° C. for one hour. Resulting DNA was purified and amplified by PCR using Q5U DNA polymerase (NEB, M0597) and paired end sequenced using an Illumina sequencer. Reads were mapped to the genome using a standard 4base mapping algorithm (BWA) and data were analyzed using the orientation aware algorithm as described in Baum C, et al., Nucleic Acids Res. 2021 Nov. 8; 49(19).). Only the first reads in the pairs were analyzed.

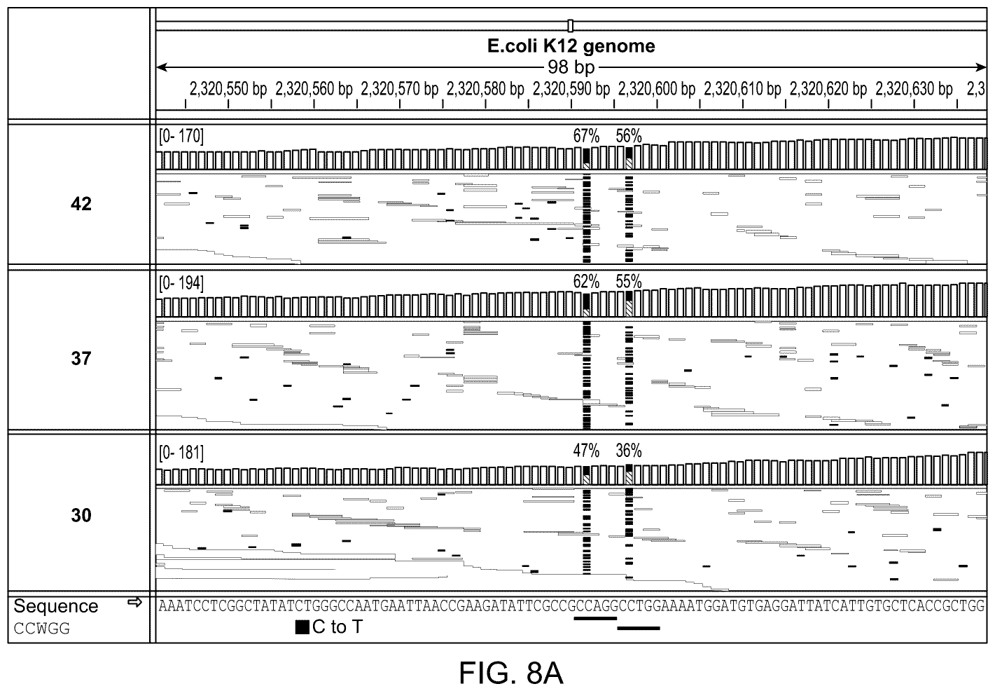

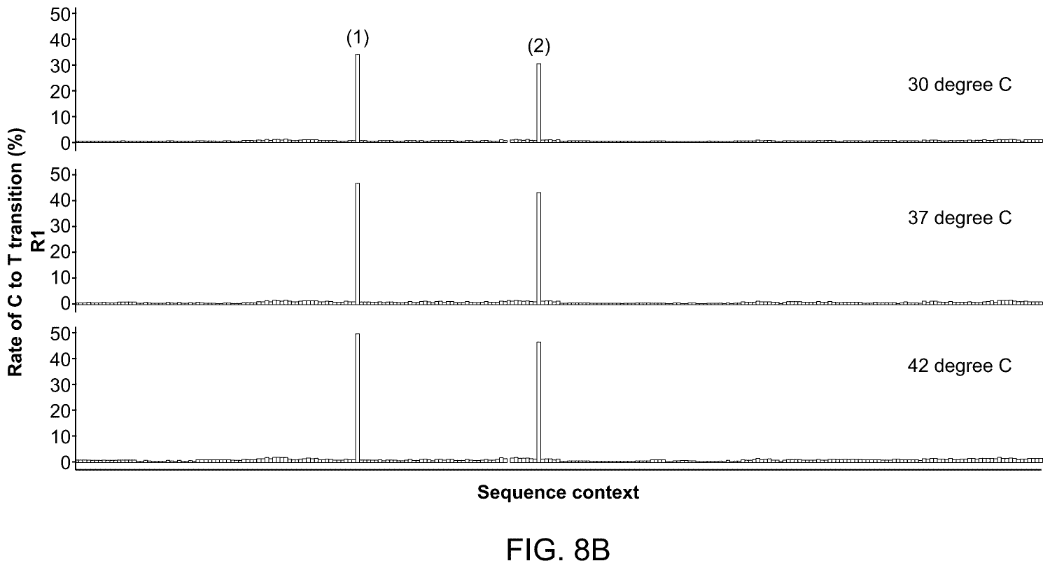

Upon deamination by the B5 deaminase, the target mC will be converted to a T. Thus, detecting a change from C to T (relative to the reference sequence) indicates the initial presence of a mC. In FIG. 8A, C to T transitions are indicated by dark blocks within the sequence representation. The C to T transition rates were quantified at two example genome loci (CCAGG and CCTGG, which are underlined at the bottom of FIG. 8A). The efficiency of C to T conversion varies from 36% to 67%, with increasing conversion rates linked to increasing incubation temperatures. The overall C to T transition rates across the genome are represented in FIG. 8B. Most of the C to T transition is observed in CCAGG (1) or CCTGG (2) context. Deaminase was tested at temperatures ranging from 30° to 70° C. and observed activity lower or equal to 55° C. (data not shown).

The results show that the majority (>95%) of the 5mC detected (reflected as C to T variants on the first read in pair) occurs at C-2 of the CCWGG sequence. This indicates that the deamination is specific to 5mC relative to C.

### Example 7. Detecting 5mC in an Epigenetic Sequencing Workflow Using Methylcytosine-Selective Deaminase

Libraries are prepared from genomic DNA following the library kit manufacturer's recommendation. Resulting DNA is treated with a methylcytosine-selective deaminase described herein. The deaminated DNA is optionally further treated with USER enzyme to chop up fragments containing uracil resulting from background deamination of unmethylated cytosine.

Libraries can be optionally amplified using standard polymerase or directly sequenced using paired-end reads. Bioinformatics analysis for detection of genetic and epigenetic information from the raw sequencing reads is performed as follows: reads are mapped to the reference genome and separated according to the first or second in pair. All reads are used to identify non-C to T or non-G to A variations using standard variation algorithm (GATK). Only the second in pair reads are used for C to T variations and only the first in pair reads are used for G to A variations. For methylation identification: the genomic positions for which variations are found (see above) are eliminated from the methylation call. Then, the first in pair reads are used for methylation calls on the forward C and the second in pair reads are used for methylation calls on the reverse strand C. Accordingly, the fraction of C to T events on the first of pair reads correspond to the methylation level.

### Example 8. Detecting 5hmC in an Epigenetic Sequencing Workflow Using a Deaminase Mixture

This example describes detecting 5hmC specifically using both a methylcytosine-selective deaminase described herein and a 5hmC specific (or 5mC/5hmC specific) deaminase together with an enzyme that protects 5hmC (e.g., carbamoyl-transferase or BGT).

Libraries are prepared from genomic DNA following the manufacturer's recommendation and resulting DNA is treated with BGT or carbamoyl-transferase or both. Subsequent treatment with a cocktail of two deaminases with complementary activity (C and 5mC) will convert C and 5mC to U and T respectively, and the 5hmC can be identified as resistant to deamination.

### Example 9. Detecting 5mC in an Epigenetic Sequencing Workflow Using Methylcytosine-Selective Deaminase and Other Enzymes

This example describes detecting 5mC using a combination of methylcytosine-selective deaminase described herein and an enzyme that protects 5hmC (e.g., carbamoyl-transferase or BGT).

Libraries are prepared from genomic DNA following the manufacturer's recommendation and resulting DNA is treated with BGT or carbamoyl-transferase or both to protect 5hmC. Resulting DNA is treated with the methylcytosine-selective deaminase. DNA can be optionally treated with USER to eliminate fragments containing uracil from deamination of unmethylated cytosine. Libraries can be optionally amplified using standard polymerase or directly sequenced using paired-end reads. Analysis can be done as described in Example 7, leading to the identification of 5mC only.

### Example 10. Detecting Different Forms of Methylcytosine Using Deaminase Combinations and a Hairpin Adaptor Workflow

A deaminase combination can be used for epigenetic sequencing of selected forms of mC, using more than one round of deamination. For example, to detect 5mC and 5hmC, the DNA sample is pretreated with BGT to protect 5hmC. Then a methylcytosine-selective deaminase described herein is used to deaminate 5mC to produce a first deaminated product. This results in conversion of 5mCs to Ts, while 5hmC is not converted. C is not substantially converted or USER treatment is employed as described above.

A “Methyl-SNP-seq” workflow can then be used (see, e.g., Yan et al, Genome Res. 2022; gr. 277080.122). A hairpin adaptor is ligated to the first deaminated product, and a free 3′ end is enzymatically generated in a double-stranded region of the hairpin adaptor. The free 3′ end is extended in a reaction mix containing a strand-displacing or nick-translating polymerase, dGTP, dATP, dTTP and 5mCTP to produce an extended strand. Cs in the extended strand will be 5mCs. The reaction product is then deaminated using bisulfite or a deaminase that is not selective for 5mC (such that 5mC is not deaminated) to produce a second deaminated product. Reads are mapped to the reference genome as described in Yan et al. The bases are identified using the schema shown in Table 4.

TABLE 4
Base calling schema for sequence reads using a methylcytosine
deaminase in a first round of deamination

| | | | Base of |
|---|---|---|---|
| | Sequenced base | Sequenced base | reference |
| Identity of base | (read 1) | (read 2) | genome |
| C | T | C | C |
| 5mC | T | T | C |
| 5hmC | C | C | C |

In another approach to detect 5mC and 5hmC, the DNA sample is pretreated with BGT to protect 5hmC. Then a deaminase that preferentially deaminases C (e.g., a double-stranded deaminase described in WO2023097226) is used to deaminate C to produce a first deaminated product. This results in conversion of Cs to Us, while 5hmC and C are not converted. A hairpin adaptor is ligated to the first deaminated product. A free 3“end is extended in a reaction mix containing a strand-displacing or nick-translating polymerase, dGTP, dATP, dTTP and CTP to produce an extended strand. The reaction product is then deaminated using a methylcytosine-selective deaminase to produce a second deaminated product. Reads are mapped to the reference genome as described in Yan et al. The bases are identified using the schema shown in Table 5.

TABLE 5
Base calling schema of sequence reads using a methylcytosine
deaminase in a subsequent round of deamination

| | | | Base of |
|---|---|---|---|
| | Sequenced base | Sequenced base | reference |
| Identity of base | (read 1) | (read 2) | genome |
| C | T | T | C |
| 5mC | C | T | C |
| 5hmC | C | C | C |

### Example 11. Detecting Methylcytosine in the Absence of a Reference Sequence

This example describes obtaining genome assembly and methylation data in an instance for which a reference genome is either unavailable or imperfect. The libraries of the genomic DNA and sequencing procedures are carried out as described in an Example above, depending on whether the goal is to obtain data for 5mC and 5hmC, 5hmC, or 5mC, respectively. During the assembly process, an algorithm is used that considers only the second/first in pair reads when a C to T or G to A transition is specifically observed in the first/second in pair reads, respectively. Such an algorithm can be a modification of an existing assembly algorithm or the process can be done in two steps: the first step involves assembling the reads using a standard assembler such as SPADE. The second step involves mapping the reads to the new assembly and correcting the position for which a C to T or G to A transition is specifically observed in the first/second in pair reads but not the second/first in pair read respectively. This corrected de-novo assembly negates the need for a reference genome and methylation can be assessed using the same dataset following Example 7.

### Example 12. Detecting Methylcytosine in Samples with Low Allelic Fractions

This example describes identifying methylation at very low allelic fractions. Genomic DNA from two differentially methylated cell lines is mixed at various ratios, such as 0:100, 0.1:99.9, 1:99, 5:95, 20:80, and 50:50, in triplicates. Libraries and sequencing procedures are carried out as described in in the above Examples, depending on whether the goal is to obtain data for 5mC and 5hmC, 5hmC, or 5mC, respectively. To identify differentially methylated regions, a standard data analysis pipeline (e.g., edgeR) can be utilized. The analysis aims to determine the lowest allelic fraction required for the detection of differentially methylated regions.

### Example 13. Detecting Methylation in Chromatin

This example describes obtaining methylation in open chromatin in fixed/frozen tissue. A tissue is subjected to chromatin accessibility assays (DNase-seq, FAIRE-seq, NICE-seq, ATAC-seq PMID: 25473421) that isolate accessible locations of a genome. DNA is extracted and subjected to deamination as described in an Example above (to obtain 5mC/hmC, 5hmC and 5mC respectively). Methylation and open chromatin information can be identified simultaneously using NGS sequencing and standard analysis tools.

### Example 14. Detecting Methylation at a Transcription Factor Binding Site

This example describes determining methylation at transcription factor binding sites in fixed/frozen tissue/cells. The cells are subjected to chromatin immunoprecipitation (ChIP) assays with sequencing (ChIP-seq, Cut and Run, Cut and Tag, NEED-seq) following the standard protocol. DNA is extracted and subjected to deamination as described in an Example above (to obtain 5mC/hmC, 5hmC and 5mC respectively). Methylation and transcription factor binding site information can be identified simultaneously using standard analysis tools.

### Example 15. Preparation of an RNA-Seq Library for Identification of 5mC/5hmC Using Methylcytosine-Selective Deaminase

This example describes preparation of an RNA-seq library for the identification of 5mC and/or 5hmC in RNA at base resolution using a methylcytosine-selective deaminase described herein. Total RNA or polyA selected RNA is treated with a deaminase described herein. RNA sequencing is then performed using standard procedure for RNA sequencing. In parallel another RNA sequencing is performed on the same starting material with the omission of the deaminase treatment. Sequencing reads are mapped to the reference transcriptome and methylated sites (5mC and 5hmC) are identified using C to T variation present only in the treated sample.

### Example 16. Preparation of an RNA-Seq Library for Identification of 5mC Using Methylcytosine-Selective Deaminase

This example describes preparation of an RNA-seq library for the identification of 5mC in RNA at base resolution using deaminase and carbamoyl transferase. The process employs pretreatment with carbamoyl transferase to protect 5hmC. Methylated sites (5mC only) are identified using C to T variation present only in the treated sample, whereas 5hmC remains C due to the protection.

Identification of 5hmC can be performed using the comparative sequencing result from Examples 15 and 16.5hmC sites show C to T variation in Example 15 but remain C in Example 16.

### Example 17. Detecting Methylase Specificity Using Methylcytosine-Selective Deaminase

This example describes identifying methylase specificity in bacteria or microbiomes. Libraries are prepared. Assembly can be done following Example 12. Reads are mapped back to the assembled genome(s) and the methylase specificity can be identify using published algorithms (see, for example, Baum C, Lin Y C, Fomenkov A, Anton B P, Chen L, Yan B, Evans T C, Roberts R J, Tolonen A C, Ettwiller L. Rapid identification of methylase specificity (RIMS-seq) jointly identifies methylated motifs and generates shotgun sequencing of bacterial genomes. Nucleic Acids Res. 2021 Nov. 8; 49(19):e113. doi: 10.1093/nar/gkab705. PMID: 34417598; PMCID: PMC8565308). More specifically, a list of the C positions for which a large fraction of the first in paired reads are read as a T (list A) is compared to a list of randomly generated C positions in the genome (list 2). Over-represented context(s) in the list 1 compare to 2 represent the methylase specificity(es) of the respective bacteria in the microbiome.

### Example 18. Tagmentation Using Methylcytosine-Selective Deaminase

This example describes a variety of approaches for identifying cytosine methylation using tagmentation.

In one exemplary approach, genomic DNA is fragmented and tagged with an adaptor (e.g., a biotin-P7 adaptor) using Tn5 transposase. The fragments are deaminated using a methylcytosine-selective deaminase described herein followed by sequencing as described herein.

In another approach, tagmentation can be performed on nuclear lysates or during chromatin precipitation to characterize DNA methylation at transcription factor binding sites (see, e.g., Lhoumaud, P., Sethia, G., Izzo, F. et al. EpiMethylTag: simultaneous detection of ATAC-seq or ChIP-seq signals with DNA methylation. Genome Biol 20, 248 (2019).). DNA is purified, deaminated using methylcytosine-selective deaminase, optionally PCR amplified, and sequenced. The resulting sequence provides DNA methylation information (e.g., presence of 5mC) and amount of a sequence that is present is an indication of chromatin openness and/or transcription factor binding.

### Example 19. Use of Methylcytosine-Selective Deaminase in Hybridization Based Assays

This example describes the detection of 5mC in genomic DNA using a combination of a methylcytosine-selective deaminase described herein and a DNA amplification/probe hybridization method, such as loop mediated isothermal amplification (LAMP) or PCR based assay. Genomic DNA extracted from, for example blood, is treated with the methylcytosine-selective deaminase followed by an optional USER treatment to cut Us resulting from any deamination of Cs. The selected assay is then performed on treated and untreated samples using primers that are specific to the deaminated version of the genomic DNA, to detect 5mC as conversion to T in the genomic DNA sample. In addition to LAMP and PCR, any detection technology that requires hybridization of one or several specific probe(s) can be used.

### Example 20. Identification of Methylated Motifs in Bacteria Using 5mC Specific Deaminase

This example describes preparation of a sequencing library of the bacteria Neisseria meningitidis for obtaining genetic and epigenetic sequence information using NGS sequencing. This example also describes the de-novo identification of methylated primary motifs and the identification of low allelic frequency methylation at certain context similar to the primary motifs, indicative of star activity of the methylase.

The genomic DNA of Neisseria meningitidis was combined with control DNA from XP12 phage (5mC), T4gt phage (5hmC) and Lambda (C), and treated as described in Example 4 and the corresponding library was deaminated using purified B5 deaminase. The deaminated library was amplified using Q5 polymerase using the manufacturer recommendation (NEB M0544S) and the resulting amplified library was sequenced using an Illumina sequencing system. A total of 64,022,543 paired-end reads were obtained for which 99.26% of the reads mapped to either Neisseria meningitidis or the control genomes demonstrating a high mapping rate despite deamination. For each position on the genomes, the C to T allelic fraction in read 1 was calculated and 0.25% C to T was found to reduce the false positive and negative rate to 0.11% and 0.27% C to T respectively. Thus, the limit of detection when using B5 deaminase in this experiment was estimated at 0.25% C to T allelic fraction. Limit of detection can be lowered by using USER treatment (Uracil DNA glycosylase and Endonuclease VIII) (New England Biolabs, product no. M5505S). Since the conversion rate at 5mC for this experiment was estimated to be 40%, the limit of detection of methylation when using B5 deaminase in this experiment was estimated to be 0.62% methylation levels.

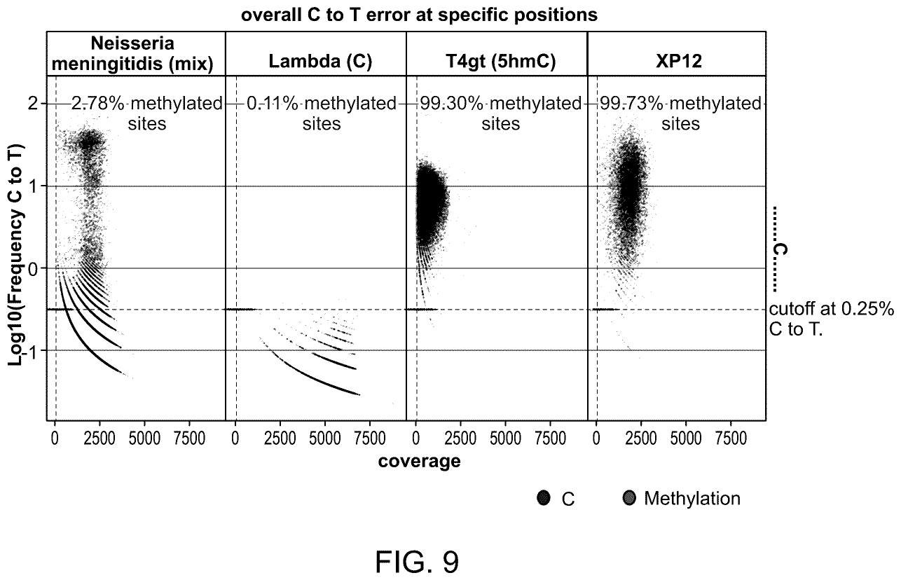

Using a cutoff of 0.25% C to T as the detection limit for methylation, it was found that 0.11, 99.3 and 99.73% of C positions are methylated in Lambda, T4gt and XP12 respectively, as indicated by FIG. 9. A total of 2.78% of C positions are methylated in Neisseria meningitidis indicating methylation at specific sites.

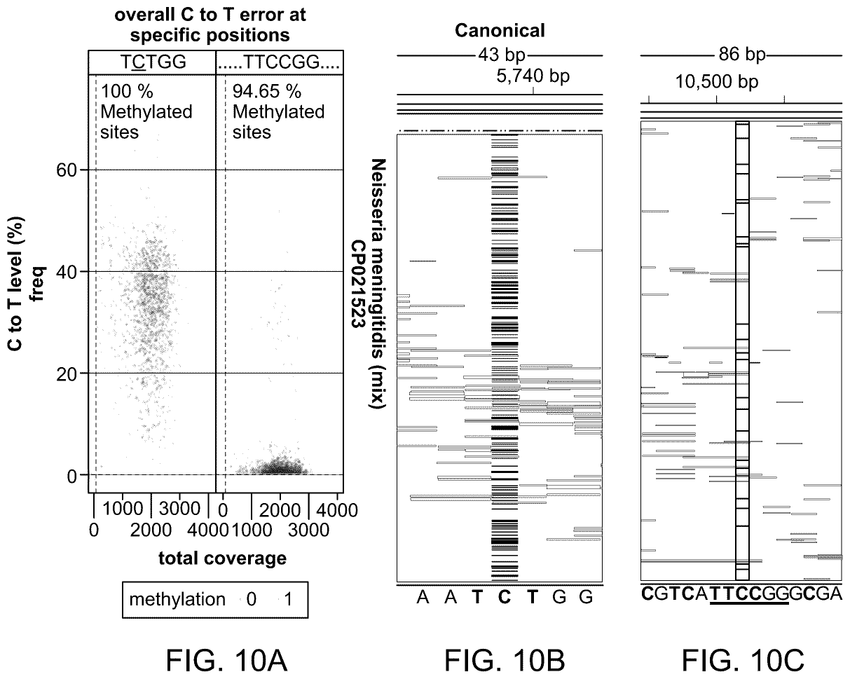

Next, the methylated motifs in Neisseria meningitidis were identified using a modified version of the Mosdi pipeline implemented here (Baum C, Lin Y C, Fomenkov A, Anton B P, Chen L, Yan B, Evans T C, Roberts R J, Tolonen A C, Ettwiller L. Rapid identification of methylase specificity (RIMS-seq) jointly identifies methylated motifs and generates shotgun sequencing of bacterial genomes. Nucleic Acids Res. 2021 Nov. 8; 49(19):e113. doi: 10.1093/nar/gkab705. PMID: 34417598; PMCID: PMC8565308). Seven motifs were found to be methylated in Neisseria meningitidis: TCTGG; CCAGA; GGNNCC; GCACGC; GCGTGC; CCTGG; CCAGG. In addition to the methylated motifs, a number of off-by-one methylated motifs were found (FIG. 10A-10C). Methylation at these sites is at low allelic fraction (<5%) and can be the result of star activity of the methylase.

FIG. 10A-10C shows allelic frequency of C to T transition in Neisseria meningitidis relative to read coverage at TCTGG and TTCCGG sites (with C being the interrogated base). The vast majority of TCTGG and TTCCGG sites are methylated but the TTCCGG sites show a lower allelic fraction that is methylated indicating a star activity site of the methylase.

### Example 21: Demonstration of Specific Detection of 5mC in 5mC/5hmC Mixed Genomes Using T4-BGT Treatment

This example describes the use of B5 deaminase for specific detection of 5mC by using BGT treatment to block deamination of 5hmC. The substrate was a mixture of T4_147 DNA (a T4 Δagt Δbgt strain; contains 5hmC fully replacing C); XP12 (contains 5mC fully replacing C) and dcm⁻ phage Lambda genomic DNA (contains C). T4-BGT treatment was performed prior to B5 deamination to convert 5hmC to 5ghmC (thereby protecting 5hmC from deamination). The BGT reaction was performed at 37C for 1 h and cleaned up with Ampure beads. The DNA was then heat denatured and deaminated with B5 deaminase at 42° C. for 1 h. PCR amplification was performed by Q5U to control for non-specific dC deamination.

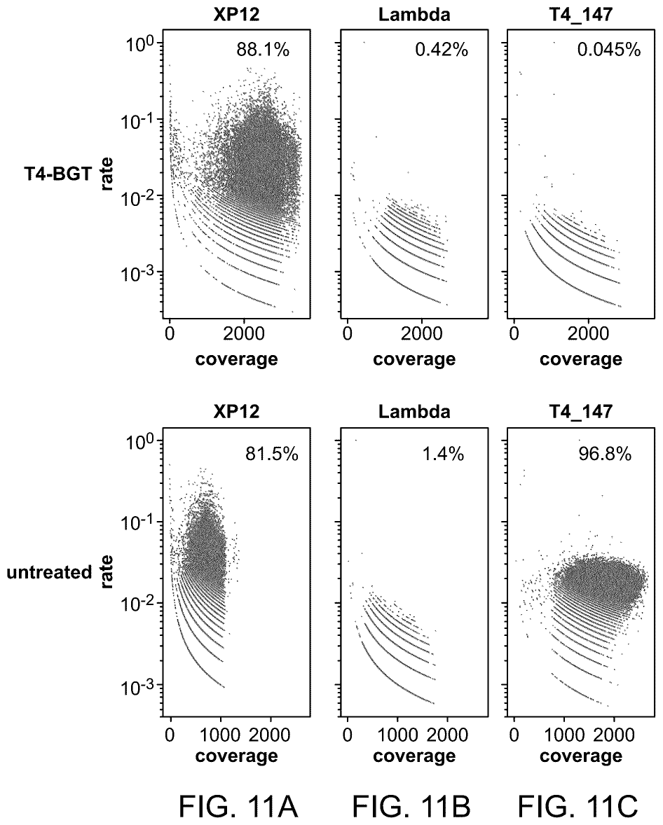

The results show that with BGT treatment, 5hmC in T4_147 Genome was protected from B5 deamination (only 0.045% of C were detected as methylated while the untreated control, methylation is 96.8%, FIG. 11A-11C). Conversely, detection of 5mC in XP12 genome was unaffected with 88.1 and 81.5% methylated sites with or without BGT treatment. This experiment demonstrates that protected (glucosylated) 5hmC is not a substrate for B5 deaminase. Thus, B5 deaminase can be used for 5mC specific detection for samples in which both 5mC and 5hmC are present by protecting 5hmC.

### Example 22: Linear Relationship Between 5mC/C Ratio and C to T Transition Rate

This example describes the design of an assay to assess whether the rate of C to T transition observed in sequencing reads can be linearly correlated with the methylation level. For this, F1 and R1 complementary were mixed at equimolar concentration to obtain a double stranded fully methylated CpG site at the highlighted position. Independently, F2 and R2 complementary oligos were mixed at equimolar concentration to obtain a double stranded fully unmethylated CpG site at the highlighted position. Next, double stranded oligo F1R1 and F2R2 were mixed adequately to obtain a 0% (no F1R1 oligo), 0.5%, 1%, 2%, 5%, 10%, 20%, 40%, 80% and 100% (no F2R2 oligo) allelic fraction of methylated sites.

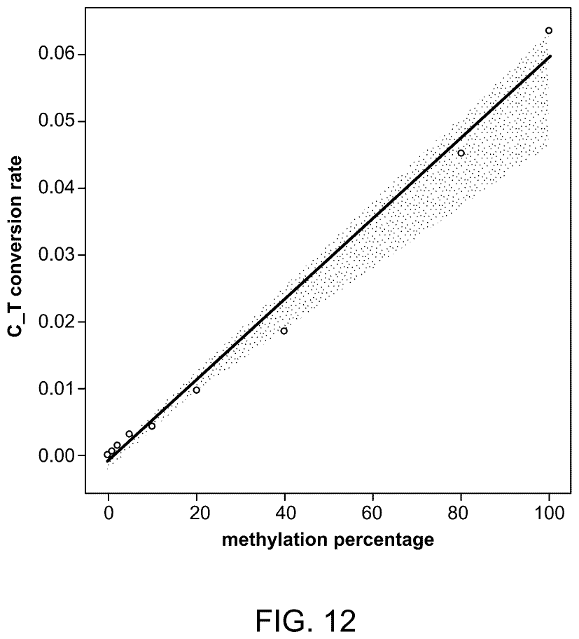

6 pmol of the mixture oligo was treated using B5 deaminase at 42C for 1 h with enzyme to substrate rate equal to 40:1 before library preparation and Illumina sequencing. Deamination signals were detected at base resolution based on the frequency of C to T variant (FIG. 12). The fraction of C to T deamination indicative of deamination shows a linear relation with methylation level demonstrating the potential of B5 deaminase to be used for high sensitivity and quantitative diagnostic purposes.

F1 (SEQ ID NO: 48):
5′GGACACAGGGGTTGCCCAGGCTACAGATC5mCGGGTTCAGGCCCAGC
CGTGGGGACTGTTCACCTCTGAGAGGGCAGCCCTGCCCCGCGGCCCTGG
CCCTGCCCTGCACCCGGCTGGGTCTCATCTGACACGC
R1 (SEQ ID NO: 49):
5′GCGTGTCAGATGAGACCCAGCCGGGTGCAGGGCAGGGCCAGGGCCGC
GGGGCAGGGCTGCCCTCTCAGAGGTGAACAGTCCCCACGGCTGGGCCTG
AACC5mCGGATCTGTAGCCTGGGCAACCCCTGTGTCC
F2 (SEQ ID NO: 50):
5′GGACACAGGGGTTGCCCAGGCTACAGATCCGGGTTCAGGCCCAGCCG
TGGGGACTGTTCACCTCTGAGAGGGCAGCCCTGCCCCGCGGCCCTGGCC
CTGCCCTGCACCCGGCTGGGTCTCATCTGACACGC
R2 (SEQ ID NO: 51):
5′GCGTGTCAGATGAGACCCAGCCGGGTGCAGGGCAGGGCCAGGGCCGC
GGGGCAGGGCTGCCCTCTCAGAGGTGAACAGTCCCCACGGCTGGGCCTG
AACCCGGATCTGTAGCCTGGGCAACCCCTGTGTCC

### Example 23: Features of Methylcytosine-Selective Deaminases

Fifty phage deaminases likely to selectively deaminate mC modifications were identified and compared with 217 phage deaminases unlikely to selectively deaminase mC. The criteria to distinguish likely from unlikely included an enzymatic screening to enrich for DNA carrying cytosine modification and computation based on an enrichment score to quantify the degree of cytosine modification of each DNA sequence as described in Yang W, Lin Y C, Johnson W, Dai N, Vaisvila R, Weigele P, Lee Y J, Corrêa I R Jr, Schildkraut I, Ettwiller L. 2021 Nov. 8; 10:e70021. doi: 10.7554/eLife. 70021. PMID: 34747693; PMCID: PMC8670742.

It was observed that 34% (17 out of 50) of deaminases characterized as likely to selectively deaminate mC contained the following amino acids (positions are stated relative to SEQ ID NO:1): S at position 28; S at position 48; Y at position 51; G at position 53; and N at position 81, whereas only 7% (17 out of 215) of deaminases characterized as unlikely to selectively deaminate mC contained these amino acids. Results of activity testing were consistent, in that the C4, F4, B5, contain this sequence motif and similarly, the E10, E8, and C73 deaminases contain (relative to SEQ ID NO:1), S at position 28; S at position 48; F at position 51; G at position 53; and N at position 81. Therefore, these amino acid positions are useful for predicting mC deaminating activity of phage deaminases.

Sequence alignments of about 82 methylcytosine-selective deaminases and suspected methylcytosine-selective deaminases yielded the following longest distances between defined amino acids of the B5 deaminase signature sequence: 33 amino acids between S and S, and 66 amino acids between G and N of the motif S-S-G-Y/F-N.

### Example 24: B5 Deaminase Activity on a Solid Support

This example describes the capability of four methylcytosine-selective deaminases disclosed herein to selectively deaminate 5mC, using EcoR1 cleavage as a readout in a capillary electrophoresis method.

About 1 nmole of B5 deaminase in a total volume of 40 μL in storage buffer (20 mM Tris, pH 7.5, 100 mM NaCl, 1 mM TCEP) was incubated at 4° C. for 1 hour with end-over-end mixing with 10 μL of NEBExpress® Ni-NTA Magnetic Beads that had been pre-equilibrated in buffer containing 20 mM sodium phosphate (pH 7.4), 300 mM NaCl, and 10 mM imidazole. Beads were washed once with 100 μL in buffer containing 20 mM sodium phosphate (pH 7.4), 300 mM NaCl, and 20 mM Imidazole. Beads were then washed twice with 100 μL of buffer containing 20 mM sodium phosphate (pH 7.4) and 300 mM NaCl. Beads were resuspended in 19 μL of activity buffer (50 mM BIS-Tris, pH 6.5), 1 μL of 5 μM 5mC substrate (250 nM oligo) was added to the resuspended beads, and the solution was incubated at 42° C. overnight with gentle agitation at 300 rpm. The reverse complement oligo (GCGTAAGCCCATGAATTCTGCATCGGTGTAT) (SEQ ID NO:43) (2-fold concentration) was subsequently annealed to the reaction (heat to 95° C. and cool to room temperature in EcoRI buffer). Ni magnetic beads were removed after the solution had cooled to room temperature, and the annealed solution was then digested with EcoRI (conditions following manufacturer protocol, 1H digestion 100 nM oligo concentration). The mixture was subsequently treated with Proteinase K and diluted to 1 nM dsDNA for compatibility with the capillary electrophoresis system.

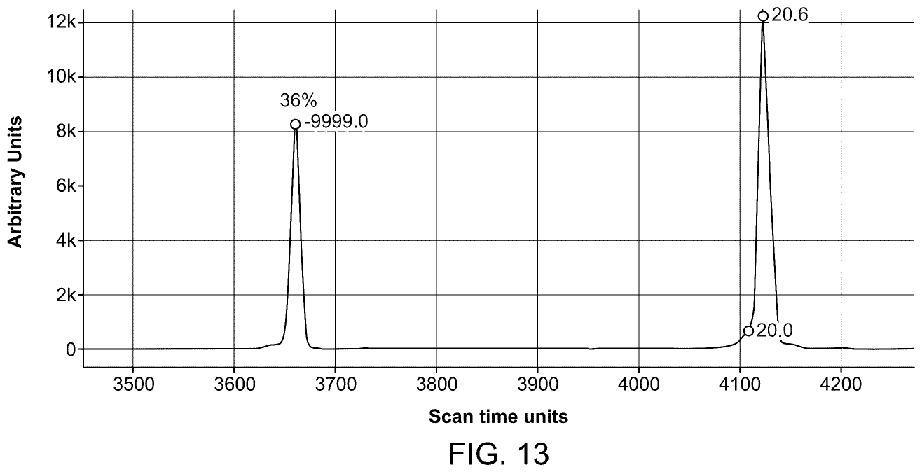

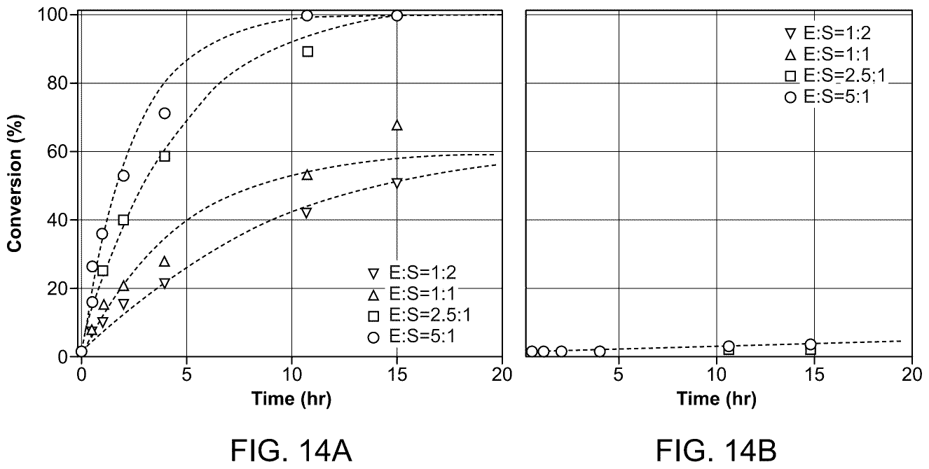

Oligos containing GAA(5mC)TC show 36% digestion indicating a % deamination of 5mC by B5 deaminase when immobilized on Ni-NTA magnetic beads of 36% (FIG. 13).

### Example 25. Genomic Neighborhoods of Phage Methylcytosine-Selective Deaminases

MetaGPA is a framework to link viral genotypes with specific phenotypes directly from high-throughput sequencing of environmental metaviromes (Yang, Weiwei, Yu-Cheng Lin, William Johnson, Nan Dai, Romualdas Vaisvila, Peter Weigele, Yan-Jiun Lee, Ivan R. Corrêa Jr, Ira Schildkraut, and Laurence Ettwiller. 2021. “A Genome-Phenome Association Study in Native Microbiomes Identifies a Mechanism for Cytosine Modification in DNA and RNA.” eLife10 (November). https://doi.org/10.7554/eLife.70021.1). MetaGPA datasets containing contig pools from viral fractions of a metagenomic library derived from a costal seawater microbiome that was either untreated or treated with restriction endonuclease known to cut canonical DNA but not C-5 cytosine modified DNA were studied. Gene neighborhood profiles of sequences annotated as phage dCMP deaminases identified in these metaGPA datasets were constructed within a maximum of +/−3 kb genomic region. Annotation was performed using hidden Markov model with Pfam domains (Pfam domain model names are shown in parentheses below). The results indicated that the top five genes that co-localize with predicted dCMP deaminases were thymidylate synthase (thymidylat_synt), MafB19 like deaminase (MafB19-deam), Phosphoribosyl-ATP pyrophosphohydrolase (PRA-PH), DNA polymerase A (DNA_pol_A) and 3′-5′ exonuclease (DNA_pol_A_exo1). 104 out of the 149 (70%) annotated dCMP deaminase genes found in modified contigs co-localized within 6 kb (+/−3 kb) of a thymidylate synthase gene. In contrast, this colocalization was rarely found (17 out of 277, 6.1%) in genomes predicted to contain canonical DNA. Multiple sequence alignments of these deaminase-associated thymidylate synthase homologs revealed amino acids in thymidylate synthase active sites that correlate with a preference for dCMP over dUMP (Liu, L., and D. V. Santi. 1992. “Mutation of Asparagine 229 to Aspartate in Thymidylate Synthase Converts the Enzyme to a Deoxycytidylate Methylase.” Biochemistry31 (22): 5100-5104).

As an example, B5 deaminase was identified within a contig assembled from the metagenomic sequence of the viral fraction in a wastewater treatment plant microbiome. This contig also encodes a thymidylate synthase, with its active site predicted in-silico and experimentally validated to utilize dCMP as its substrate and producing 5mdCMP. The closest homolog to the thymidylate synthase adjacent to B5 is from the Xanthomonas phage Xp12, which is characterized by the complete substitution of cytosine with 5-methylcytosine in its genomic DNA (Farber, M. B., and M. Ehrlich. 1980. “Bacteriophage XP-12-Induced Exonuclease Which Preferentially Hydrolyzes Nicked DNA.” Journal of Virology 33 (2): 733-38.) indicating that the phage which B5 is originated from similarly contains complete substitution of cytosine with 5-methylcytosine.

For seven of the thymidylate synthase genes found in modified contigs containing methylcytosine-selective deaminases the synthesis of 5mdCMP was confirmed for five of them, indicating that some methylcytosine-selective deaminases that are in-cis with these thymidylate synthases are relevant to the biosynthesis of modified cytidines. Accordingly, this study has identified a previously unrecognized signpost for identifying a methylcytosine-selective cytosine deaminase.

### Example 26. Deamination of Modified Nucleotide Species

The ability of B5 deaminase to deaminate 5-formylC (5fC), 5-carboxyC (5caC) and glucosylated 5hmC (5ghmC) in single-stranded DNA oligonucleotide context was tested and quantified using an LC/MS assay.

As is shown in Table 6, B5 deaminase selectively deaminates the 5mC or 5hmC on single-stranded DNA over C, whereas the deamination is low on the C5-modified 2′-deoxycytidines 5fc, 5caC and 5gmC.

TABLE 6

| Nucleotide species | Deamination conversion | |
|---|---|---|
| 5mC | 53% | |
| 5hmC | 36% | |
| 5fC | 2% | |
| 5caC | 2% | |
| 5gmC | 2% | |
| C | 2% | (average) |

### Example 27. Deamination of Double Stranded DNA

This example shows that the B5 deaminase has greater activity on single-stranded genomic DNA compared to double-stranded genomic DNA.

B5 deamination activity was tested directly on bacteriophage XP12, T4, T4gt and lambda double stranded genomic DNA and little activity was observed. When this experiment was performed using denatured XP12 genomic DNA prior to deamination, a notable activity of the B5 deaminase was observed, indicating that B5 acts preferably on single stranded DNA (ssDNA).

DNA was denatured by NaOH plus heating to generate pseudo-single-stranded DNA. For each genomic DNA experiment set, pseudo-single-stranded DNA gives the most deamination conversion on the modified nucleotides, as shown in Table 7.

TABLE 7

| | Deamination conversion | Deamination conversion |
|---|---|---|
| Nucleotide species | (no NaOH nor heat) | (NaOH and heat) |
| 5mC (XP12) | 4% | 35% |
| 5hmC (T4gt) | 9% | 27% |
| 5gmC (T4) | 3% | 6% |
| C (lambda) | 3% | 6% |

### Example 28. Epigenetic Sequencing Using Methylcytosine-Selective Deaminases

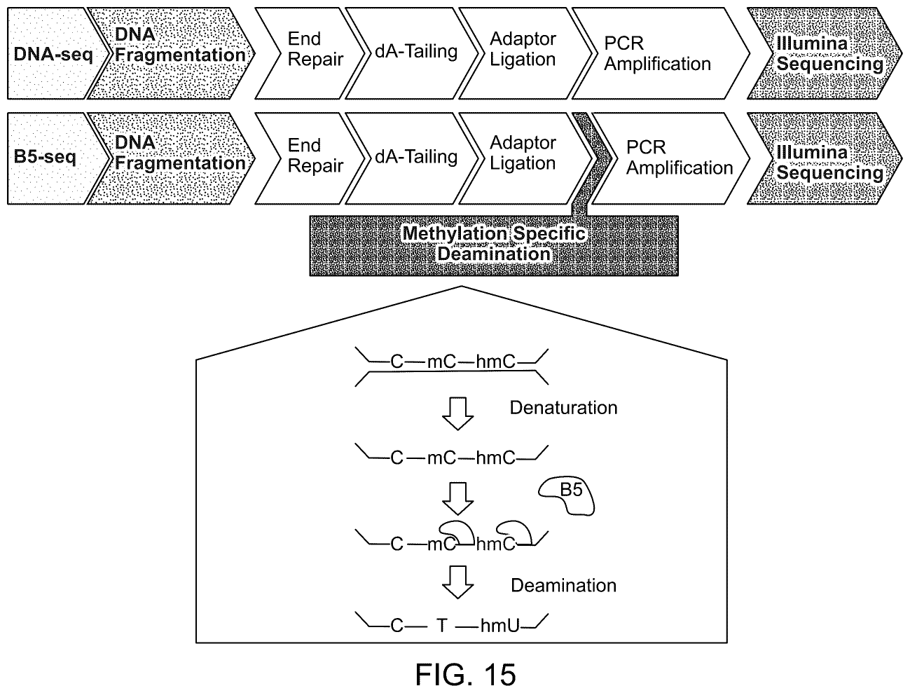

Next-generation sequencing (NGS) was used to further characterize 13 methylcytosine-selective deaminases that demonstrated 5mC selectivity in the CE assay described in Example 5. As is depicted in FIG. 15, in this approach, genomic DNA is fragmented, and a library is constructed by ligating standard Illumina adapters prior to denaturation. 5mC conversion to T is achieved by incubating the denatured DNA with the deaminases. Following amplification, the libraries are sequenced on the Illumina platform. Although the depicted workflow shows adaptor ligation prior to methylcytosine-selective deamination, the deamination step can be performed prior to adaptor ligation, in which case single-stranded ligation would typically be used after deaminating single stranded DNA.

The sample was a mixed genomic DNA pool derived from the XP12, T4gt, and Lambda bacteriophages, which represent fully 5-methylcytosine (5mC)-modified, fully 5-hydroxymethylcytosine (5hmC)-modified, and unmodified cytosine (C) genomes, respectively. Additionally, genomic DNA from E. coli K12 strain DHB4, which harbors 5mC in the CCWGG context (where W=A or T, with methylation occurring on the internal cytosine), was sequenced, providing a genome with mixed cytosine modification states. This sample is referred to as the “mix-control”. In order to capture all deamination events including deamination of C, the uracil tolerant engineered polymerase Q5U was used to amplify all deamination products (Wardle, Josephine, Peter M. J. Burgers, Isaac K. O. Cann, Kate Darley, Pauline Heslop, Erik Johansson, Li-Jung Lin, et al. 2008. “Uracil Recognition by Replicative DNA Polymerases Is Limited to the Archaea, Not Occurring with Bacteria and Eukarya.” Nucleic Acids Research 36 (3): 705-11). Paired reads were mapped to the composite genomes using BWAmeth. To evaluate possible deamination preferences, the deamination rates (% deamination) at each genome were calculated for each of the 256 NNCNN contexts (with N=A, T, C or G).

In agreement with CE assay results, the deaminases exhibited higher catalytic activity towards 5mC and 5hmC compared to cytosine. The highest deaminase activity on 5mC of the deaminases tested in this experiment was observed in the B5, E10, and F7 deaminases. While all active enzymes displayed varying degrees of activity across different sequence contexts, they did not exhibit strict context specificity but some had preferred contexts (see Example 34). A phylogenetic tree of the amino acid sequences of deaminases displaying activity in either CE or NGS assays was constructed, and, together with landmark sequences from diverse organisms ranging from viruses to humans, indicated that this activity can be found in clades together with enzymes displaying C to U deamination. This mixed distribution of 5mC specific methylcytosine-selective deaminases across multiple clades suggests mixed lineages, where no single ancestral event gave rise to the family of sequences having the 5mC specificity.

### Example 29. Methylcytosine-Specific Deaminase Sequencing

Short-read sequencing library preparation conditions were adapted for B5 deamination to develop methylation-specific cytidine deaminase sequencing or mSCD-seq (FIG. 15). 20 ng of the control gDNA-mix sample was fragmented and ligated with standard sequencing adapters and the resulting libraries were denatured at 85° C. for 10 min and treated with B5 deaminase at 42° C. for one hour prior to the final PCR amplification and sequencing using Illumina platform. Reads were mapped to reference genomes using BWA-Meth or BWA-mem. Methylation levels were called using MethylDackel, with methylation and non-methylation levels inverted. The inversion is useful because, unlike bisulfite or EM-seq which convert C to U (and thus read as T), mSCD-seq converts 5mC to T and C remains C. Once inverted, the output of MethylDackel is compatible with downstream methylation analysis pipelines.

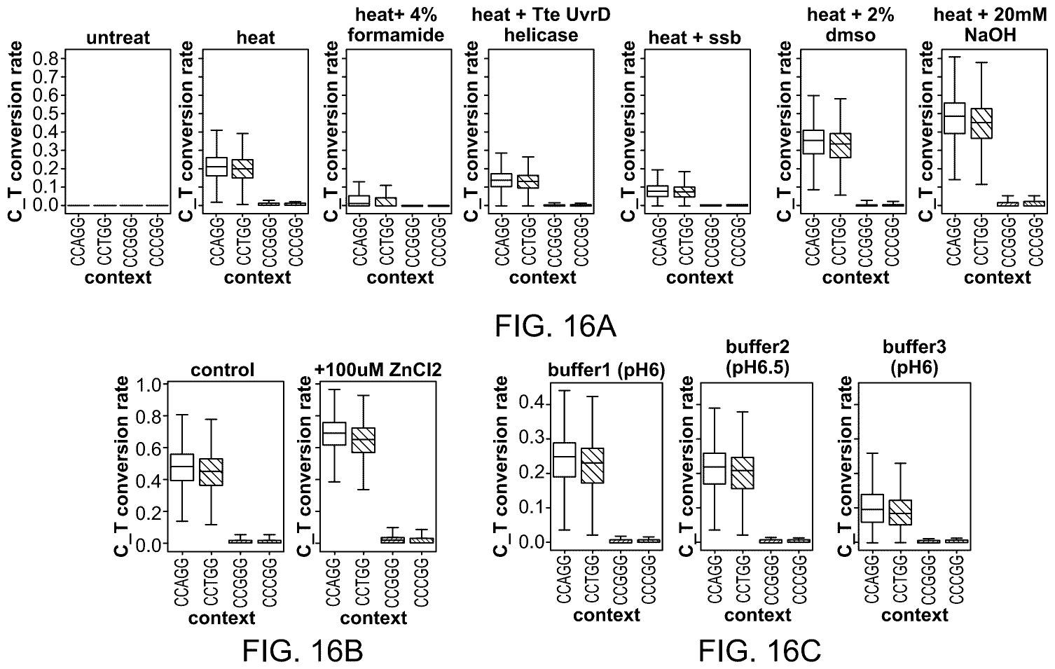

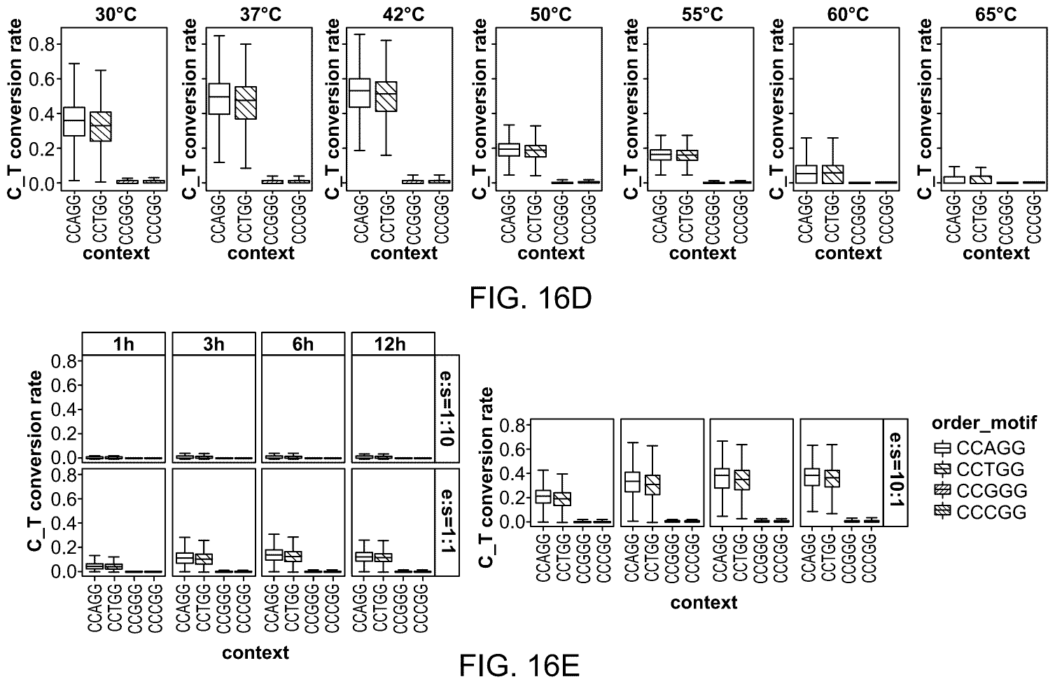

Several denaturation conditions were tested, including the addition of NaOH, DMSO, formamide, helicase and ssDNA binding protein (FIG. 16A). Deamination rates (C_T conversion rate) of every mC in DHB4 genome CmCNGG context are shown. The combination of heat and 0.02 M NaOH demonstrated higher performance. The addition of Zn²⁺ enhanced the activity of B5; 100 μM ZnCl₂ was added to the deamination reaction (FIG. 16B). Deamination rates (C_T conversion rate) of every mC in DHB4 genome CmCNGG context are shown in FIG. 16B. Factors such as pH, reaction buffer, reaction temperature, time of incubation and enzyme:substrate ratio were also examined (FIGS. 16C-E). Effect of pH buffer in reaction study involved Buffer 1: Bistris pH6+1% non-ionic surfactant; buffer 2: Bistris pH6.5; buffer 3: Bistris pH6+100 mM NaCl+10 mM MgCl2. Deamination rates (C_T conversion rate) of every mC in DHB4 genome CmCNGG context are shown in FIG. 16C. Effect of temperature was assessed. Deamination rates (C_T conversion rate) of every mC in DHB4 genome CmCNGG context are shown in FIG. 16D. Effect of incubation duration and enzyme: substrate ratio in reaction was assessed. Deamination rates (C_T conversion rate) of every mC in DHB4 genome CmCNGG context are shown in FIG. 16E.

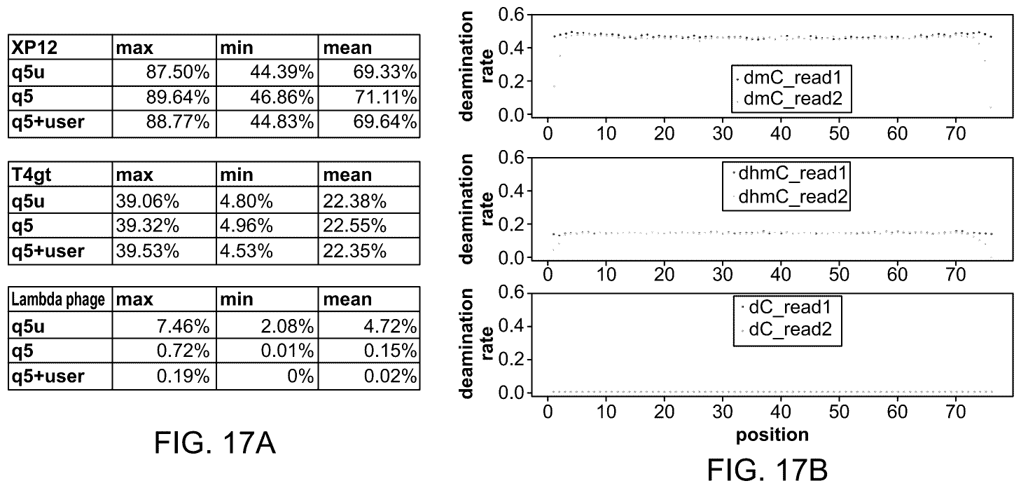

Deamination levels were higher in 5mC than 5hmC, which is consistent with prior LC/MS observations on ssDNA oligos. Using XP12 data, the deamination rate ranged from 44.4% to 87.5% with a mean value of 69.3% depending on the NNCNN sequence context. Using T4gt bacteriophage genomic DNA, deamination of 5hmC ranged from 4.8% to 39.1% with a mean value of 22.4% respectively (FIG. 17A). Deamination was even across the sequencing reads (FIG. 17B).

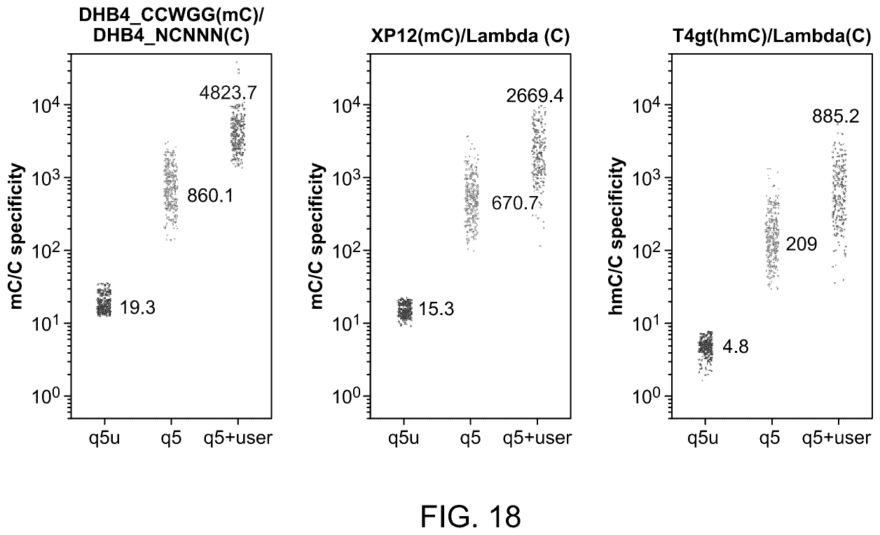

On E. coli genomic DNA, B5 selectively deaminated mC with an average C to T conversion of 84.7% in CCWGG motif while non-specific deamination at C was 3.7%. The specificity ratio of deaminating mC to C was 19.3 fold (FIG. 18A). This difference increases to an average 860 fold when the sample is amplified with Q5 instead of Q5U and to an average 4823 fold when the sample is treated with USER prior to amplification with Q5. The same analysis was repeated using the phage XP12 genomic DNA and the ratio of deaminating mC to C was 15.3 fold, 670.7 fold and 2669.4 fold when the sample was amplified by Q5U, Q5 and USER-Q5 respectively (FIG. 18B). The same analysis was repeated using the phage T4gt genomic DNA and the ratio of deaminating 5hmC to C was 4.8 fold, 209 fold and 885.2 fold when the sample was amplified by Q5U, Q5 and USER-Q5 respectively (FIG. 18C).

In addition to 5mC, prokaryotes often contain 4mC and 6 mA at specific sites in their genomic DNA (Blow, Matthew J., Tyson A. Clark, Chris G. Daum, Adam M. Deutschbauer, Alexey Fomenkov, Roxanne Fries, Jeff Froula, et al. 2016. “The Epigenomic Landscape of Prokaryotes.” PLoS Genetics 12 (2): e1005854). To assess the behavior of B5 deaminase on those commonly found prokaryotic DNA modifications, mSCD-seq was performed on control spiked-in DNA together with genomic DNA from Natrinema pallidum BOL6-1, which is known to express both 6 mA and 4mC methyltransferases producing 4mCTAG and C6mATTC, GTAYT4mCG and CAGYA6mAC (DasSarma, Priya, Brian P. Anton, Satyajit L. DasSarma, Fabiana L. Martinez, Daniel Guzman, Richard J. Roberts, and Shiladitya DasSarma. 2019. Genome Sequences and Methylation Patterns of Natrinema Versiforme BOL5-4 and Natrinema Pallidum BOL6-1, Two Extremely Halophilic Archaea from a Bolivian Salt Mine. Microbiology Resource Announcements8 (33). https://doi.org/10.1128/MRA.00810-19). It was observed that deamination levels at 4mC-methylated CTAG and GTAYTCG contexts are nearly at the level of sequencing error, being at least ten times lower than in unmethylated contexts. In contrast, unmethylated CTAG and GTAYTCG contexts exhibit deamination levels comparable to other contexts within the lambda genome. Taken together, these results indicate that B5 deaminase does not deaminate cytosines containing a methyl group at position 4. To investigate whether B5 is capable of deaminating 6 mA the rate of A-to-G transitions in 6 mA-modified contexts was examined. The observed A-to-G transition frequency (^{˜}0.00003) was consistent with standard sequencing error rates, indicating that B5 lacks deaminase activity toward adenine or its derivative, 6 mA. Overall, when applied to prokaryotic genomic DNA—where 6 mA, 4mC, and 5mC are the predominant modifications—mSCD-seq selectively identifies 5mC modifications, as neither 4mC nor 6 mA are substrates for deamination by B5.

Deamination of C, 5mC, and 5hmC produces U, T and 5-hydroxymethyluracil (hmU) respectively. To selectively reduce the background deamination of C, DNA polymerases containing a uracil recognition pocket, such as those found in archaeal family B DNA polymerases (Connolly, Bernard A. 2009. Recognition of Deaminated Bases by Archaeal Family-B DNA Polymerases. Biochemical Society Transactions 37 (Pt 1): 65-68.), can be used. Alternatively, or in combination, dU can be removed prior to amplification using combination of Uracil DNA Glycosylase (UDG)(Lindahl, T., S. Ljungquist, W. Siegert, B. Nyberg, and B. Sperens. 1977. DNA N-Glycosidases: Properties of Uracil-DNA Glycosidase from Escherichia Coli. The Journal of Biological Chemistry 252 (10): 3286-94.) and DNA glycosylase-lyase Endonuclease VIII, also known as USER treatment Bitinaite, Jurate, Michelle Rubino, Kamini Hingorani Varma, Ira Schildkraut, Romualdas Vaisvila, and Rita Vaiskunaite. 2007. USER Friendly DNA Engineering and Cloning Method by Uracil Excision. Nucleic Acids Research 35 (6): 1992-2002.).

To demonstrate the elimination of dU resulting from C deamination, mSCD-seq was performed on the mix-control sample library that was either amplified with Q5U or Q5 with or without prior treatment with USER. A loss of material in the library was observed in Q5 and Q5+USER amplification compared to Q5U. Quality control metrics also show bias when dU is eliminated from the library using USER/Q5. Notably, base composition in Q5 and Q5+USER datasets show bias for AT rich regions and insert sizes tend to be smaller than the Q5U dataset. This discrepancy arises because the USER-Q5 step eliminates preferentially fragments that are longer and are GC-rich.

An average of 19 fold difference between deamination of 5mC compared to C for sample amplified using Q5U was observed. This difference increases to an average 860 fold when the sample is amplified with Q5 instead of Q5U and to an average 4823 fold when the sample is treated with USER prior to amplification with Q5. Similarly, amplification using Q5U, Q5 and USER+Q5 leads to an average 5 fold, 210 fold and 885 fold in apparent deamination levels of 5hmC compared to C respectively. These results indicate that the selectivity for 5mC/5hmC can be significantly enhanced by applying a USER treatment to the deamination reaction. When combining post-deamination USER treatment with Q5 polymerase, the absolute C-to-T transition rate at unmethylated cytosine reaches values as low as 0.2%, which closely approaches, if not matches, the baseline error rate of Illumina sequencing. With these conditions, mSCD-seq has specificity for detecting 5mC/5hmC. This specificity allows detection of residual methylation levels in purported unmethylated lambda control at CCWGG sites. Unmethylated lambda genomic DNA—a commonly employed control for unmethylated DNA in methylome sequencing—is typically derived by propagation of the phage in a dcm-E. coli host; a mutant strain bearing the dcm-6 allele, which contains a nonsense mutation in the dcm gene. Thus, dcm-6 strains can retain residual dcm methyltransferase activity. To demonstrate the presence of residual methylation at CCWGG sites in dcm-6 strains, mSCD-seq was performed on E. coli strains containing either wild-type dcm (dcm+), a nonsense mutation in the dcm gene (dcm-6), or a complete deletion of the dcm gene (Δdcm). Results revealed strong and weak methylation at CCWGG for the dcm+ and dcm-6 strains, respectively, and no detectable methylation in the Adcm strain. Equivalent results were obtained using Tet-assisted PacBio sequencing (Clark, Tyson A., Xingyu Lu, Khai Luong, Qing Dai, Matthew Boitano, Stephen W. Turner, Chuan He, and Jonas Korlach. 2013. “Enhanced 5-Methylcytosine Detection in Single-Molecule, Real-Time Sequencing via Tet1 Oxidation.” BMC Biology11 (January):4.). BMCBiology11 (January):4.).

These experiments demonstrate the sensitivity of 5mC detection using mSCD-seq in combination with USER treatment prior to amplification with Q5. Notably, detection of residual methylation in the purported methylation-free lambda genomic DNA (i.e. lambda DNA produced in an E. coli host carrying the dcm-6 amber mutant allele of dcm) emphasizes sensitivity in identifying even minute amounts of methylation. USER+Q5 treatment was used for subsequent experiments unless the intrinsic cytosine deamination rate is required, in which case, amplification using Q5U was performed.

Based on the present studies, the mSCD-seq method is compatible with standard DNA library preparation protocols, incorporating only a one-hour (or less) deamination reaction step prior to the final PCR enrichment. Unlike other methylation detection techniques that necessitate specialized adaptors (e.g., bisulfite-seq and EM-seq), mSCD-seq libraries can be prepared using conventional Y-shaped or loop-shaped adaptors.

mSCD-seq selectively converts around 80% of 5mC. At this activity level, the background C to U conversion is minimal (around 1-4%) leaving the majority of unmethylated cytosines unchanged. Importantly, deamination of 5mC results in a T, which is a canonical base in DNA. This distinction between T and U allows mSCD libraries to be treated with USER, eliminating the remaining background deamination of the C while leaving intact the product of deamination of 5mC. mSCD-seq can 5mC with detection of methylation as low as 0.01%. Accordingly, mSCD-seq is well-suited for disease diagnostic assay, particularly in scenarios such as early cancer detection, where samples often exhibit low tumor purity. mSCD-seq can be used in combination with USER treatment for the identification of non-CpG methylation in the brain and residual methylation in circulating DNA (see below). mSCD-seq provides a positive readout of methylation, unlike BS or EM-seq, where the background C must be fully converted to uncover methylation. Any remaining non-conversion in BS or EM-seq can lead to confusion, as it can be erroneously interpreted as methylation.

mSCD-seq selectively converts 5mC (and 5hmC), leaving the majority of unmethylated cytosines unchanged. Given that 5mC represents only about 1-2% of cytosines in the human genome, deamination using B5 predominantly preserves the original DNA sequence. As a result, the converted DNA behaves much like regular DNA, allowing for PCR amplification, target enrichment, and other standard molecular biology techniques that are not feasible with traditional BS or EM-seq. Furthermore, sequencing B5-treated genomic DNA enhances the ability to accurately map reads to the human genome and facilitates variant calling alongside methylation analysis. This is an important feature of B5 specifically for multi-omics applications for which many types of information are layered on top of one another. mSCD-seq deamination results in a canonical T, with a low amount of cytosines deaminated to uracil (approximately 1-4%). As a result, amplification can be conducted using standard polymerases. B5 treatment is generally compatible with standard molecular biology protocols and subsequent enzymatic treatments that can be hindered or blocked by uracil, such as DNA or RNA polymerases, restriction enzymes, and genome editing enzymes. B5 also is expected to have a stronger genome editing capacity because the deaminated 5mC is a T.

Integrating UMIs into the mSCD-seq protocol can further mitigate the impact of sequencing errors by enabling consensus-based error correction. As a result, mSCD-seq coupled with UMIs is expected to identify minute levels of residual methylation, pushing the detection threshold even lower. This combination makes mSCD-seq a powerful tool for epigenetic applications that require ultra-sensitive detection of DNA methylation.

### Example 30. Protecting 5hmC in NGS Sequencing

Treatment of 5hmC with T4 phage β-glucosyltransferase (BGT) has been previously used for the protection of the β-glucosyl-5-hydroxymethylcytosine against deamination by APOBEC. BGT specifically transfers a glucose moiety to the 5-hydroxymethylcytosine (5-hmC) residues in double-stranded DNA, creating a beta-glucosyl-5-hydroxymethylcytosine (5-GhmC) that is resistant to deamination by APOBEC.

Experiments were performed to investigate whether BGT could fully protect 5hmC from B5 deamination. Genomic DNA from lambda (C) Xp12 (5mC) and T4gt (5hmC) was treated with or without BGT prior to deamination. The resulting deaminated libraries were amplified with Q5U to capture all deamination events and paired-end sequenced to 5 million reads. Reads were mapped to the reference genomes using BWA-Meth and analyzed for imbalance of C to T transition in Read1 compared to read 2. Such imbalance has been previously shown to represent deamination rather than variants or stochastic sequencing errors compared to the reference sequence (Chen, Lixin, Pingfang Liu, Thomas C. Evans Jr, and Laurence M. Ettwiller. 2017. “DNA Damage Is a Pervasive Cause of Sequencing Errors, Directly Confounding Variant Identification.” Science355 (6326): 752-56). and is a particularly sensitive measure for low level deamination.

In the untreated sample, T4gt genomic DNA which has fully replaced C with 5hmC, exhibited a notable deamination level after B5 treatment, consistent with B5 activity on 5hmC (FIG. 11A-11-C). In contrast, BGT treatment prior to B5 treatment almost completely abolished deamination in T4gt genomic DNA indicating that the BGT treatment has prevented B5 from deamination. Deamination levels for both Xp12 and Lambda is not affected by BGT treatment . . . .

This finding aligns with our earlier results using oligonucleotide substrates and confirms that beta-glucosyl-5-hydroxymethylcytosine is not a substrate of B5 deaminase. From the LC-MS assay, it was demonstrated that 5fC and 5caC are not substrates of B5. Thus, BGT treatment prior to mSCD-seq deamination would specifically reveal 5mC in genomes (such as the human genome) that contain a mixture of C, 5mC, 5hmC, 5fC, and 5caC.

### Example 31. Human Methylome Sequencing

mSCD-seq was performed on 100 ng of human NA12878 genomic DNA, for which numerous published reference methylation datasets are available, generated using methodologies such as EM-seq, bisulfite sequencing, and Nanopore sequencing. Human genomic DNA was spiked with standard DNA to control for C and 5mC conversion such as XP12, T4gt and pUC19 plasmid methylated in CpG contexts. Resulting libraries were either amplified by Q5U or USER treated followed by Q5 amplification and sequenced. Because deamination is restricted to 5mC sites, reads derived from mSCD-seq have mostly retained the same DNA sequence and can be mapped using standard mapping algorithms. Thus, reads were mapped to a composite genome containing hg38 reference genome and the controls using either BWA-mem (Li, Yan-Hua, Hai-Feng Hou, Zhi Geng, Heng Zhang, Zhun She, and Yu-Hui Dong. 2022. “Structural Basis of a Multi-Functional Deaminase in Chlorovirus PBCV-1.” Archives of Biochemistry and Biophysics 727 (September):109339.) or BWA-meth (Pedersen B. S., Eyring K., De S., Yang I. V., Schwartz D. A. Fast and accurate alignment of long bisulfite-seq reads. Arxiv. 2014:1-2.).

The strong correlation between methylation levels assessed using reads mapped with either BWA-MEM or BWA-Meth (R=0.95, p<2.2e-16) indicates that mSCD-seq-derived reads can be reliably aligned to the human genome using both mapping algorithms. The average methylation levels obtained from mSCD-seq were compared with those from EM-seq and Nanopore sequencing. The results demonstrated a strong correlation between mSCD-seq and EM-seq (R=0.91, p<2.2e-16), as well as between mSCD-seq and Nanopore sequencing (R=0.88, p<2.2e-16).

The excess of all transition and transversion types in non-CpG contexts in the first paired-end read were compared to the second paired-end read. These values were then compared with sequencing outcomes obtained through PCR-free DNA-seq (ftp://ftp.sra.ebi.ac.uk/vol1/fastq/ERR194/ERR194147/ERR194147_2.fastq.gz). The findings indicated that, across the majority of substitution classes, the profile post-B5 treatment closely resembled that of DNA-seq.

Mapping of mSCD-seq reads does not require three-letter encoding unless fully modified genomes like XP12 are being investigated. Data analysis was conducted using common BS-seq alignments and extraction tools bismark and methyldackel (with bwa-mem or bwa-meth as the mapper) <ref>. Comparisons between these tools showed strong correlation of up to 99.5%, demonstrating the feasibility and reliability of mSCD-seq data analysis using universal methylation analytical tools.

mSCD-seq method was tested for its ability to identify non-CpG methylation in human brain. As background, non-CpG methylation is carried out by the de novo methyltransferases DNMT3a and DNMT3b can accumulate in post-mitotic cells. In neurons, DNMT3a is the main de-novo methyltransferase and is believed to be responsible for the non-CpG methylation preferably in CAC context (Lee, Jong-Hun, Sung-Joon Park, and Kenta Nakai. 2017. “Differential Landscape of Non-CpG Methylation in Embryonic Stem Cells and Neurons Caused by DNMT3s.” Scientific Reports 7 (1): 11295. Lister, Ryan, Eran A. Mukamel, Joseph R. Nery, Mark Urich, Clare A. Puddifoot, Nicholas D. Johnson, Jacinta Lucero, et al. 2013. “Global epigenomic reconfiguration during mammalian brain development.” Science 341 (6146): 1237905. Mallona, Izaskun, loana Mariuca Ilie, Ino Dominiek Karemaker, Stefan Butz, Massimiliano Manzo, Amedeo Caflisch, and Tuncay Baubec. 2021. “Flanking Sequence Preference Modulates de Novo DNA Methylation in the Mouse Genome.” Nucleic Acids Research 49 (1): 145-57.). These results were obtained using bisulfite sequencing for which low frequency methylation such as the one found in non-CpG context can only reliably be called when the conversion is observed at statistically significantly higher levels than those that might be attributed to the artifacts caused by incomplete bisulfite conversion.

In this study, mSCD-seq in combination with USER treatment and Q5 amplification on 100 ng of total human brain. 460 million reads were mapped to the reference human genome and analyzed as described above. A motif logo that is consistent with Dnmt3A sequence preference with the two most non-CpG contexts to be methylated being TTACACC and TTCCACC.

Another feature of brain genomic DNA is high levels of 5hmC in a CpG context (Globisch, Daniel, Martin Münzel, Markus Müller, Stylianos Michalakis, Mirko Wagner, Susanne Koch, Tobias Bruckl, Martin Biel, and Thomas Carell. 2010. “Tissue Distribution of 5-Hydroxymethylcytosine and Search for Active Demethylation Intermediates.” Edited by Anna Kristina Croft. PloS One 5 (12): e15367.). As described above, BGT-mSCD-seq identifies 5mC and the comparison with the mSCD-seq should reveal the amount of 5hmC. BGT-mSCD-seq was performed in combination with USER treatment and Q5 amplification to 100 ng of the same starting material. BGT treatment is expected to result in the complete protection of 5hmC revealing 5mC. The level of 5mC obtained by BGT-mSCD-seq was compared with the level of 5hmC obtained using NEBNext® Enzymatic 5hmC-seq. Very few methylated regions have no 5hmC detected and most of the methylated regions have around 25% of 5hmC in line with the reported amount of 5hmC found in brain. Few unmethylated regions have detectable 5hmC consistent with the fact that 5hmC is the oxidative product of 5mC.

To observe residual methylation, NA12878 genomic DNA was mixed with WT-DKO DNA at a 99:1 ratio respectively. Hypomethylated CpG islands in NA12878 were selected and ranked according to the methylation levels in WT-DKO (0-30%, 30-50%, 50-80% and >80%). A gradual increase in methylation levels in the mixed sample to the target 1% in CpG islands where the WT-DKO levels are >80%.

### Example 32. Cell-Free DNA Methylation

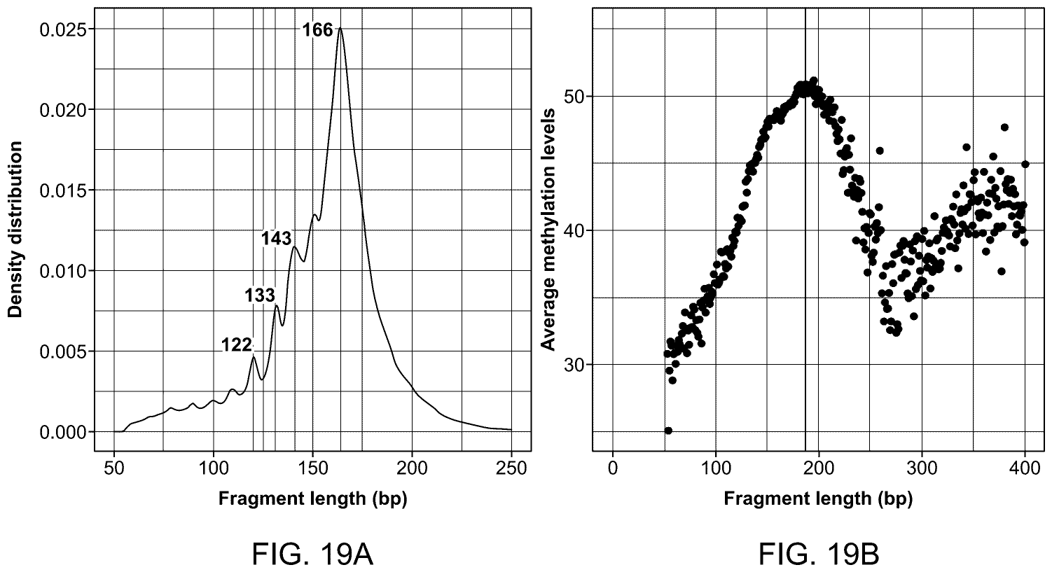

mSCD-seq was performed using cell free DNA from a healthy donor and the converted library shows the expected fragment distribution sizes. Furthermore, the methylation levels correlates with the fragment sizes as expected. cfDNA fragment length peaks at around 166 base pairs (bp), which corresponds to the combined length of DNA wrapped around a nucleosome (147 bp) plus a 20 bp linker fragment. These fragments result from nuclease degradation and their methylation patterns provide information about the tissue origins. Epigenetic marks such as DNA methylation have been used for the detection of fetal DNA, cancer or organ transplant. Besides methylation, another important epigenetic information contained in cfDNA is nucleosome occupancy. Using enzymatic conversion, recent studies have combined both epigenetic marks in a single experiment (Erger, Florian, Deborah Nörling, Domenica Borchert, Esther Leenen, Sandra Habbig, Michael S. Wiesener, Malte P. Bartram, et al. 2020. “cfNOMe-A Single Assay for Comprehensive Epigenetic Analyses of Cell-Free DNA.” Genome Medicine 12 (1): 54. Siejka-Zielińska, Paulina, Jingfei Cheng, Felix Jackson, Yibin Liu, Zahir Soonawalla, Srikanth Reddy, Michael Silva, et al. 2021. “Cell-Free DNA TAPS Provides Multimodal Information for Early Cancer Detection.” Science Advances 7 (36): eabh0534) including TAPS-seq on cf-DNA. To obtain methylation on a read level, Biscuit (Zhou, Wanding, Benjamin K. Johnson, Jacob Morrison, Ian Beddows, James Eapen, Efrat Katsman, Ayush Semwal, et al. 2024. “BISCUIT: An Efficient, Standards-Compliant Tool Suite for Simultaneous Genetic and Epigenetic Inference in Bulk and Single-Cell Studies.” Nucleic Acids Research 52 (6): e32.) was used with the following parameters: biscuit cinread -p “QNAME, QPAIR, STRAND, BSSTRAND, MAPQ, QBEG, QEND, CHRM, CRPOS, CGRPOS, CQPOS, CRBASE, CCTXT, CQBASE, CRETENTION”-g chr1 -t cg on the mapped reads. mSCD-seq was performed in replicate on cfDNA from a healthy donor, and the converted library exhibited the expected fragment size distribution (FIG. 19A). Furthermore, methylation levels were found to correlate with fragment sizes (FIG. 19B), consistent with previous findings. The average deamination at CpG sites was approximately 60%, displaying a bimodal distribution indicative of fully methylated or unmethylated states. Replicates showed high reproducibility, with a Pearson correlation coefficient of 0.968.

### Example 33. Rapid Detection of Nucleotide and Nucleobase Selectivity of Deaminases by Monitoring Ammonia Production

A method for detecting deaminase activity by monitoring ammonia production was developed/In preparation for detecting ammonia released during deamination in solution, ammonium chloride standards were prepared in B5 activity buffer (50 mM Bis-Tris, pH 6) in a total volume of 20 μL. Deaminase (2 μM) reactions with mononucleotides (500 μM) were prepared in a total volume of 20 μL in B5 activity buffer and incubated at 37° C. Deaminases used in this section were purified by FPLC on a large scale or with free IMAC resin on a small scale and dialyzed into standard storage buffer after elution with imidazole buffer. To adjust the pH to be within optimal range for ammonia detection, 0.5 μL of 1 M NaOH added to each reaction. Commercially available ammonia detection kits were screened, and a kit from Abcam performed the best (ab83360). To the pH adjusted reactions, 30 μL Ammonia Assay Buffer was then added to each sample (total volume 50 μL). The kit was then used according to the manufacturer's instructions. Color change from clear to pink in 30-60 minutes was detected on a plate reader or scored qualitatively where specified.

A colorimetric assay for ammonia was used to test whether deaminases that were eluted from MAC resin into buffers containing high concentrations of imidazole (ex. 250 mM or above) could be used directly for activity screening without the need for buffer exchange. To test assay tolerance to imidazole, ammonia detection reactions were run in final concentrations of 50 mM or 100 mM imidazole (representing the addition of 4 or 8 μL of eluted protein added to a deaminase activity assay where the final volume is 20 μL. Table 8 illustrates the quantifiable amounts of ammonia released by deamination reactions on dCTP, d5mCTP, and/or dCMP, which is greater in the presence of enzyme compared to substrates alone. Each sample was run in duplicate. Consistent with LC-MS data presented herein and the literature, deamination of dCMP by the T4 deaminase is preferred and demonstrated by an ammonia detection assay. Similarly, the preference of B5 for 5mdCTP as a nucleotide substrate, over dCTP, can be detected through monitoring ammonia release in the presence or absence of imidazole.

TABLE 8
50 mM Imidazole

| Sample | | Absorbance |
|---|---|---|
| | Ammonia concentration (μM) | |
| Blank | 0 | 0.117 |
| Std1 | 20 | 0.242 |
| Std2 | 40 | 0.303 |
| Std3 | 60 | 0.413 |
| Std4 | 80 | 0.4779 |
| Std5 | 100 | 0.525 |
| | Calculated ammonia concentration (μM) | |
| dCTP/dCMP | 25 | 0.2443 |
| d5mCTP | 29 | 0.261 |
| B5 only | 34 | 0.282 |
| B5 + dCTP | 41 | 0.312 |
| B5 + d5mCTP | 48 | 0.340 |
| T4 only | 28 | 0.259 |
| T4 + dCTP | 44 | 0.321 |
| T4 + dCMP | 90 | 0.511 |

100 mM Imidazole

| Sample | | Absorbance |
|---|---|---|
| | Ammonia concentration (μM) | |
| Blank | 0 | 0.122 |
| Std1 | 20 | 0.251 |
| Std2 | 40 | 0.340 |
| Std3 | 60 | 0.435 |
| Std4 | 80 | 0.505 |
| Std5 | 100 | 0.565 |
| | Calculated ammonia concentration (μM) | |
| dCTP/dCMP | 13 | 0.208 |
| d5mCTP | 16 | 0.221 |
| B5 only | 20 | 0.238 |
| B5 + dCTP | 25 | 0.260 |
| B5 + d5mCTP | 29 | 0.277 |
| T4 only | 11 | 0.199 |
| T4 + dCTP | 19 | 0.235 |
| T4 + dCMP | 44 | 0.3416 |

Selected methylcytosine-selective deaminases were screened for activity and selectivity using the Modified Ammonia Assay. B5 and T4 deaminases were used as controls and homolog activity was qualitatively assessed by visualizing a color change (clear to pink). Activity and selectivity were scored accordingly. No activity detected is marked by a (−) and positive activity is denoted with (+, ++, or +) Results are shown in Table 9.

TABLE 9
5mC selective deaminase activity assessed
using a colorimetric reaction

| Homolog | Activity on C | Selectivity on 5mdCTP |
|---|---|---|
| B5 | + | +++ |
| 201 | ++ | ++ |
| 210 | + | − |
| 216 | − | − |
| 219 | + | + |
| 250 | + | +++ |
| 251 | + | +++ |
| 252 | + | +++ |

### Example 34. Preferred and Least Preferred Sequence Contexts in XP12 Genomic DNA Using B5 Deaminase

MSCD-seq was performed using multiple methylcytosine-selective deaminases. The two least and two most preferred substrates for the G12, B5, E10, F7, and H3 deaminases, along with their respective deamination efficiencies (percent deamination (%)) at 5mC, were identified through short-read sequencing of XP12 genomic DNA following deaminase treatment and are shown in Table 10. The underscored cytosine (C) represents the position assessed for conversion during the deamination process. Results such as this can be used to select methylcytosine-selective deaminases for particular purposes, e.g., to target a specific 5mC and/or 5hmC in a DNA substrate, to obtain a deaminase mixture with broad context, to obtain a deaminase mixture with a context, to obtain a deaminase mixture selective for a particular type of DNA substrate (e.g., to distinguish between DNA from different species in the same sample, or for more efficient deamination of a particular type of sample). As a non-limiting example, G12 and B5 can be combined to obtain a deaminase mixture with a broad context, given that G12 has preference for CpG while B5 has preference for AT rich region.

TABLE 10
Deamination sequence context for exemplary methylcytosine-selective deaminases

| | | | 2nd to | | | | | |
|---|---|---|---|---|---|---|---|---|
| | least | | least | | most | | 2nd most | |
| | preferred | | preferred | | preferred | | preferred | |
| Enzyme | motif | % | motif | % | motif | % | motif | % |
| G12 | GCCCC | 0.90 | GCCTC | 0.94 | TACGG | 12.71 | AACGG | 11.40 |
| B5 | GGCCC | 22.51 | GGCGC | 22.69 | ATCAA | 74.58 | ATCAT | 73.46 |
| E10 | GGCGC | 11.58 | GGCCC | 12.03 | ATCAA | 40.91 | ATCAT | 40.90 |
| F7 | GGCCG | 5.51 | CGCCG | 5.81 | GTCAG | 21.99 | TTCAA | 21.93 |
| H3 | GGCCC | 1.28 | GGCGC | 1.33 | AACTT | 11.26 | AACTG | 10.95 |

### Example 35. Structural Study of Methylcytosine-Selective Deaminase Family Member

A model of m5dCMP bound to B5 deaminase was generated first by computationally folding the B5 amino-acid sequence using AlphaFold2 followed by alignment with a crystallographic structure of the dCMP bound PBCV-1 deaminase (PDB ID: 7fh4) using the align command in PyMol. PCBV-1 chains were deleted leaving behind dCMP bound in a pseudo-docked conformation in the B5 active site. Lastly, the dCMP structure was computationally replaced with 5mdCMP (PDB ID: 5CM) using the approach just described. The resulting model is shown in FIG. 20, with residues in a methylcytosine-selective deaminase signature sequence S-S-Y-G-N highlighted. Amino acid numbering is with respect to SEQ ID NO:1 with a methionine added to the N-terminus The residues constituting the S-S-G-Y-N signature sequence described in Example 23 populate the surface of an allosteric regulatory site known in other homologues to bind the enzyme product or pathway related metabolites to modulate enzyme activity (Hou, H. F., Liang, Y. H., Li, L. F., Su, X. D., & Dong, Y. H. (2008). Crystal structures of Streptococcus mutans 2′-deoxycytidylate deaminase and its complex with substrate analog and allosteric regulator dCTP·Mg2+. Journal of molecular biology, 377(1), 220-231). This site can be found by structural alignment between computationally predicted fold of a cytidine deaminase with an experimentally derived structure of a cytidine deaminase containing a nucleotide bound at this regulatory site (e.g. pdb: 7fh4). Therefore, those skilled in the art could model a candidate methylcytosine-selective deaminase using AlphaFold 2 or similar software followed by alignment with the crystallographic structure of the dCMP bound PBCV-1 deaminase (PDB ID: 7fh4) using the align command in PyMol or similar approach, to further ascertain that an enzyme containing the defined methylcytosine-selective signature sequence is likely a B5 deaminase family member.

TABLE 9
Sequence Listing

| SEQ ID NO. | Name | Amino Acid sequence |
|---|---|---|
| SEQ ID NO: 1 | B5 | NEQPTKRTQEEWDSFFLRFARDVAGMSKDPDRQVGAVVVTPDRRQLSLGY |
| | | NGFPTEVEDLPSLLADRDFKLANMVHAEVNCLRQAPFPLEGCTLYVTRFPCDR |
| | | CAERIVASGVRRVVAPAPDFGHARWGSSWLQAAGSMESAGLAITLYKGE |
| SEQ ID NO: 2 | C4 | SQKQKLKQAFMSVAYDMASVSCCERGNHACVLVSRSLRIVSVGYNGPAARL |
| | | HNGCARPLESGNCGCVHAEANAVLQPRIDHPFYAFVTAAPCEYCAQLLVNA |
| | | GVRTVVYDVVTDSGHDGLKVLTNAGVGNFQFNQFSGDWLGLGS |
| SEQ ID NO: 3 | D5 | EVFKAFTQQLATLSKCTERQVAAIITDKNLTQVYSVGINGGPKGLADCMCVID |
| | | GKYGCIHAEINALIKDKSSDEGKVMFVTLAPCKQCAAAIINAPKGFSTVYYFED |
| | | WKEDSGIKLLTSAGITVAKI |
| SEQ ID NO: 4 | E8 | ACLVSTWSKDPQRGVGALLVSPDMRQVSWGFNGFPQGIADDPALLHDSQA |
| | | KLERMVHAELNVILNSRRDVSGWTLYVTQHPCNHCALAIIQAGISMVVCPEP |
| | | KPITSKWHVSHQIAAGLLEEAGVLVRRP |
| SEQ ID NO: 5 | E10 | HERPIKRTQEEWDSFFLRFARDVASMSKDPDRQVGAVVVTPDRRQLSLGFN |
| | | GFPPGVEDLPSLLADRDFKLANMVHAEENCLRQAPFPLEGCALYVTRFPCHH |
| | | CAEKIVAARVRRVVAPAPDFGHGRWGQSWLQSAESMESAGLAITLYKGE |
| SEQ ID NO: 6 | F4 | KRPSLEDTMMDIAGALALRSTCKKLCVGAVITDKRGRILGSGYNGVPTGMTH |
| | | CIDVPCAGAQAPKGADLCEAVHAEANALLNCYDIDKATHIYVTHVPCLRCMK |
| | | TLLNTPIKTIVYYHAANAELPALRIWASANRICTQI |
| SEQ ID NO: 7 | H10 | EVFKAFTQQLATLSKCTERQVAAIITDKNLTQVYSVGINGGPKGLADCMCVID |
| | | GKYGCIHAEINALIKDKSSDEGKVMFVTLAPCKQCAAAIINAPKGFSTVYYFED |
| | | WKEDSGIKLLTSAGITVAKI |
| SEQ ID NO: 8 | H60 | SSGLVPRGSHMALAVFISDWSKDPSSKVGAVISEGNRIVSVGYNGFPRGIVDD |
| | | VNRLNDRQTRLKYTIHAELNAILQARETVRGYYLFTTHYPCSSCAQAIVQAGIS |
| | | RVYYRYSLGRWNESEAAQIFKEAEVLVKQVDIRRSDDLGG |
| | | VSAYTRVGWE |
| SEQ ID NO: 9 | B5 fusion | MGSSHHHHHHTMNEQPTKRTQEEWDSFFLRFARDVAGMSKDPDRQVGA |
| | | VVVTPDRRQLSLGYNGFPTEVEDLPSLLADRDFKLANMVHAEVNCLRQAPFP |
| | | LEGCTLYVTRFPCDRCAERIVASGVRRVVAPAPDFGHARWGSSWLQAAGSM |
| | | ESAGLA |
| | | ITLYKGEIL |
| SEQ ID NO: 10 | PBCV1 | SSGLVPRGSHMSKAEKFYSLACYHAQLFSKDPNTKVAALVIDNNNNIASVGY |
| | | NGLPRGFEETSDRWEKPMKYNYVVHAQANAIATAARNGVRLDGCSIITTLFP |
| | | CKECSKLIIQSGIRKVITSKPCKDSSWLESFSFSNEMFDECGIEVEYL |
| SEQ ID NO: 11 | C73 | SSGLVPRGSHMVSKWAKRFIQMAELVGSWSKDPSTQVGAVITKNNRIVSVG |
| | | FNGYPHGISDSAEVDERETKYLKTLHAEENAILFAKRDLDGCEIWVTHFPCPNC |
| | | AAKIIQTGISVVHCPQQTEDFLSRWGEKISLSQDMFEQAGVHVDWLPLEQLD |
| SEQ ID NO: 12 | H2 | ARPSIDQWAMYIARAVSTRASCLRRKVGCVLLNNDKRIDGTGYNGAASGLRH |
| | | CDELKSCPSDFCQTGEGRDDCMAIHAEQNALLQCSDVSEINTCYVTRSPCTVC |
| | | VKLLLNTGCKRIVYQELSSHDGAAILWLKAGRQWLSLDSAEMQAMEMLRR |
| SEQ ID NO: 13 | H3 | ESAAQTSHAACPTSLTLSTNVGNISNRDTGKKQMEKTNWDKRFMRLAIEARS |
| | | WTKGLGAGIDRGVGACVVSPTHRQFSLGYAGFPRGIDDTADRLLSPEFRANH |
| | | TVHAELNAILNASMSLEGWTIFSTECPCADCSNAIVQAGIKRVVAPSPNRNSN |
| | | WYGSQLEGAETMREAGVTIERVTLAETGVVRVGASPQERAECGCNTPDVCA |
| | | SNGCQGKKRRR |
| SEQ ID NO: 14 | F7 | IDQSAWDARGMQLADDVASWSKDPDEGVGCVLASSDYRQFAFGVNGLPS |
| | | GFPHDIVMDKQTKLKYSLHAEDNAIANAATDLRGWTAYVTKPPCLRCALALH |
| | | RAGVKRVVFPELSPASSWYHAQKEAVQFMEANGLEVYLWA |
| SEQ ID NO: 15 | E10.2 | VIIMSHTKWEGRFYTLANTVADWSKDPHKKCGVVIISPDKKEISYGYNGFPTGI |
| | | ADDERLEQKETKNELMLHAEMNAILNAKKDLTGWTLYCSQSICTQCALAIIQA |
| | | NISRVVMPRIQLHSTWYLSCTNAVKLFTEANVIIHYINENEYYV* |
| SEQ ID NO: 16 | E12 | ELMKKKIVSKTWSQRFMMMAKTISNNSKDPSTKVGSVAINPNNKQILALGY |
| | | NGFPKGILDDDRLLDKNTKYSLMVHSEMNTIFNAALNGISLNGAYLYVYGLPT |
| | | CSECAKGIIQVGIKKIFYVNTNPSKIWDEHWEKSKKMYDEVKIKYEELHEKDISI |
| SEQ ID NO: 17 | F12 | VVHKWRTRRTRGAPAHCRSPVEGVSDRRRGWTHMRVAHEYAMLSHSRKA |
| | | KVGAVIVKDDRVVSVGYNGTVSGADNTCEYTADGQYLTYESVVHAEMNAIA |
| | | FAASSGSPTNGCTMYLTLSPCFNCAKLIVQSGIVEVVYDRRWHKGGDSIAFLN |
| | | DNGVLTRAQKDLPT |
| SEQ ID NO: 18 | 206 | QNLWDSRFMELAHLIGSWSKDDKRKVGCVIVSQDNRILSTGYNGFPRQLDG |
| | | LSVIPAELKNVYTIHAEVNCMANLKAQLTEGVTVYVTYPPCLECSKMLKANIPII |
| | | RLVTSSLDAYSASKWHASMLEAKAFLTDSGVEYVCADSGYTS |
| SEQ ID NO: 19 | 207 | TKRPDFDDIYMELAVNLAKKSHCIKRHVGAVLTKDTRIISIGYNGPPEKTHNCD |
| | | EEWPEEGCPRDSKGSCSLALHAEENAILFAVKNGTEVKGATIYVTLSPCISCARL |
| | | LFASGVKKVIYLHSYAEYKGLAVDEGNAFLEKFGVTVEKFKGDFTLLNEEK |
| SEQ ID NO: 20 | 209 | SRPAFDDIFMELAVNLARRSHCIKRHVGAVLTKDTRIISIGYNGPPAGTHNCDE |
| | | EWPQTGCPRDSKGGCSLAIHAEQNAILYAVKNHTSVEGSTLYVTLSPCLACAR |
| | | MIFTMGIKRVVYLHSYAEHKGLPDDEGITFLERFGVQTEKYKGQLAVVSPLI |
| SEQ ID NO: 21 | 211 | MNELNQNIRITLEELFMAIALIVKSRSICKVNQVGVVIAPKDLRNIYSFGYNGPV |
| | | HKESHDVCNGISGNCGCLHAEINALLKVQVKDSEKIMFTTTSPCLYCAKAIIQS |
| | | GFSVIYYFEPYRDLSPIQYLKKFNIEVFQLKTDFLTKFANTLKL |
| SEQ ID NO: 22 | 215 | DNKLLITSPEKSNKIDARTISNPELIFGIVGPIGVDIDNVVASLSKSLQGVGYKSTL |
| | | IHLTKYINDSRISFDIDESSYFKRYKSLIQYGNEFRRIANSASALAGIGIAKIRERRS |
| | | ELTGVADQPALGTAYIIRQFKRAEEIDLMRSVYGRKFILISVFGSPIDRRRILIEKI |
| | | RSYDSSPKSDAQCETEAIELIDIDHNQRDDDNGQRLSDVFHLGDVFVDGIDKD |
| | | KAEGTIDRFIRAFFGDTSVSPTKDEYGLYIATAAALRSVDLSRQVGAAIFSKRGE |
| | | VITLGCNEVPKANGGTYWSDDDNQAPQRDIELGRDPNQERKNEIIYDLLDR |
| | | MFDEGLLSEKLSSVEDGQQRVDALLQNKKIKDAQLLDIIEFGRIIHAEMSAISD |
| | | AARLGRETSGAILYCTTFPCHLCAKHIVAAGMDRVVFLEPYPKSYAQKLHADSI |
| | | TFDPTDESKVLFQPFMGISPRRYRDIFEKKKRKDAAGKAIRWYNKDPVPLLED |
| | | KSGAYIENEEGALIVALKDIFQASSSSKDASADAIEG |
| SEQ ID NO: 23 | 223 | RPSWDKIWIEFARNIAKRSYDPRFQVGSVIVTEDNCQVLAVGYNGNYAGGPN |
| | | KVESEIPGESGFIHAEINALIKLDYNNPKKKKLYLTLAPCRMCAKAIVNAGIAEVI |
| | | YEEPYRDMSGVQLLQDCGIKVRAF |
| SEQ ID NO: 24 | 226 | VRPNVKEYMINIAFATSERATCVRRKVGCVLTDINNHILSTGYNGVPKGVTHCI |
| | | DRPCIAASSSSGTNLEGCKAIHAEVNAVAHCHDIQSVYAAYCTTLPCIHCIKLLL |
| | | ATNCQVIYYNQEYPHSLAKELWIDSGRELEQI |
| SEQ ID NO: 25 | 245 | KHDLRYMKLAKEVSTWSKDPSTKIGVVVIGTKGQVLSQGFNGFPRGIEDSDER |
| | | LNNREVKYEYVVHGEMNAIFNATYNGVSLDGATMYVYNLPICSECAKGIIQV |
| | | GIKRVVVLEADRGVRMRWIDSSKLSTDMFKESGVELTCLHTQL |
| SEQ ID NO: 26 | 246 | SSVGRPPWDEIWMGFAHSIARRSYDPRYQVGAVIVTKENTQVLAVGYNGNY |
| | | SGGPNEVESIEPGNSGMIHAEINALLKCDYNIRGKKIMYVTLSPCKMCAKAMI |
| | | NGGIKEVVYDEEYRDLSGVELLMSAGVAVRRYTT |
| SEQ ID NO: 27 | 251 | TEGGFQKWDKRFMRLACEARTWVKGPDLGVGACVVSPDNRGFSLGYSGLP |
| | | RGMKDTNDRITETEFKDHHMVHAELNAILNASRSVVGWTLYATTHPCSHCA |
| | | GAIIQAGIKRVVAPMPPLHSDSRWAPSWAHARMAFKEAGVIFFPYEGDLPC |
| | | D |
| SEQ ID NO: 28 | 252 | KTTQAEWDRRFLRLALEVRSWVKGSDLGVGAVIVDADRRPVSFGYSGLPRG |
| | | MCDDTPLLTDPEFKDRHMVHAELNAILNASGPLRNCTIYVTKAPCAQCCSAII |
| | | QSGITRVVAPHPDDGRWKANQAAGRMALREAGVQWQGTKSLEVEGL |
| SEQ ID NO: 29 | 011 | PKTLKSKDDLVRSVMLLARTWAYSRSKDPKTKVGAAIYTIAGNVHLGYNGFPK |
| | | NVPDWHDVWHELDPHINKHDLVIHAEANAIRKALKSGDSLGGAIMCVTLFP |
| | | CIGCIKQCASVGLTQIIYADTKSKTPEAMLIADALGITLTPYTPGGLADHA |
| SEQ ID NO: 30 | 014 | VKKIMNNFEWLTDYRLKKISPDTLLLMMKQAKNARGSSYSPYSKFKVGCSILS |
| | | EGGLVTCGTNFENQLYSKTVHAEVNAIGQMITMRERLLKCVFIASSNGVKPCV |
| | | DCRKVISEFCDQDIPIVLINTTTDQVKIHTIYELAPTFFKLGQLKLL |
| SEQ ID NO: 31 | 018 | QSTRDSKERYFLKIAALVATRSTCPRRSVGCVIVNKHNHIKATGYNGVPRGYA |
| | | HCIDTHCGGQNAASSKGLSSCMATHAEQNALLQCDDTMDIHTIYVTTSPCVT |
| | | CSKLIANTSCKRVVYSEEYADRTGIEMLNKLAIGVYYERIHD |
| SEQ ID NO: 32 | 036 | VHPHCTGVQVMLVLIEGTDFMGKTHLAKQFQERFGLGYTHLSKPDEDFDYCS |
| | | GYLAMMKDHHVFDRFHLGTLVYGSDLGCYKHTFDAIKQSKVNKSLKLRNALLI |
| | | LMYESNEDGFRTRLAGNDRAEMYNKDQIVAANESFLSQAITMRAFAELPNVF |
| | | LYDVSQGWLEFDELLDRIKLETHWRPEIESVHRLEAQIQQSCQYRHVVAVAFD |
| | | EYGQRIDHANNRTREGEICGKLSDTGPCGCVHAEDWLLSRLYVGRQSARIRTI |
| | | ALSCSPCSKCAQLIVDNGHVEFVVYGELYERDTRGVEILNEAGIKTLQA |
| SEQ ID NO: 33 | 052 | NKFDRKYLRMAKIWSENSHSKRLKVGALIVKDGSIISDGYNGTPSGFDNTCEN |
| | | DNNETLPEVLHAEANAITKIAKSHNSSEGATIYVLVSPCLSCSKLIIQAGIKRVVY |
| | | NTKYRLQDGIKLLEKAGVVIDEIEDL |
| SEQ ID NO: 34 | 054 | CRRAEKLLTFARFAKELSLLATCKRNGVGCVICDPLFTHVYSLGYNGWPRGFD |
| | | HDRCVKTNDPCGCVHAEINACLKLDTSRSDLICIVTEVPCIRCSQAIIQTHRIQR |
| | | VVVYQPTYPTTRGPDLLKEAGLQYDQM |
| SEQ ID NO: 35 | 059 | EPSKKHDLYMRIADAVALSSHCKRSKVGCIIVKNDNIIGIGYNGTPSGFENDCE |
| | | CNNTTKEEVLHAESNAITKCAKSTISSDGSIMYCTLMPCFNCAKLIIQSGIKQVI |
| | | YKDDYRNKSGIKLLTKANIEVNRYE |
| SEQ ID NO: 36 | 060 | YRMYTQLQALRRLSKCADKQVACIITDVEDNILSVGVNRVFNCVDCTKTGSKV |
| | | GCLVSHAEEAALANLARTYEAKRAYLTLYPCENCQRVLDPVVDEIIVFGKQHK |
| | | NCVIDPNKIILVPDLSKELWGTNGEQKQLSVITSELAELIKVITDFFYRRNERATK |
| | | IEEVLDEIVDVELMTAVLLDLISARYDESVLFELAKIKEAKLGKIKRRLADGTIKRD |
| | | SPYS |
| SEQ ID NO: 37 | PRW294 | GSNERPIKRTQEEWDRFFLRFARDVAGMSKDPDRQVGAVVVTPDRRQLSLG |
| | | YNGFPPGVEDLPSLLADRDFKLANMVHAEENCLRQAPFPLEGCTLYVTRFPC |
| | | HHCAEKIVAAGVRRVVAPAPDFGHARWGQSWLQSAGLMESAGIAITLYKGE |
| SEQ ID NO: 38 | PRW311 | TQDKWDRRFMRLAKEVATWSKDPDRGVGAVIVSPDRRQVSWGYNGFPRGI |
| | | ADDPERLNDKELKNQLMVHAELNAILNASRDVTGWTLYVTKHPCSHCASAII |
| | | QAGITRVVCPEPDPADSRWHESHQLARKLLEEAGVEVTHFEPDECA |
| SEQ ID NO: 39 | PRW313 | DQSKWDARFMQLADDVASWSKDPDKGVGAVLVSPDKRQFSFGYNGLPSGF |
| | | PDDVVMDKETKNRYTLHAEENAIANAAQDLRGWTLYVTKAPCLRCALAIHRA |
| | | GITRVVFPELDPSSSWYASQREAVEFLEAAGVEVARFKPCA |
| SEQ ID NO: 40 | PRW314 | TMDKWDRRFMRLALEVRSWVKGPDRGVGAVIVSPDRRQVSFGYGLPRGM |
| | | ADDEERLTDPEFKDRHMVHAELNAILNASRSVRGWTLYCTKAPCAHCCSAIIQ |
| | | AGITRVVSPSPDDSRWHRSQLEGREALREAGVEVERMEQEEAA |
| SEQ ID NO: 41 | 41 | TGTCCGATAGACT(5mC)TACGCA |
| SEQ ID NO: 42 | 42 | TGTCCGATAGACT(5hmC)TACGCA |
| SEQ ID NO: 43 | 43 | GCGTAAGCCCATGAATTCTGCATCGGTGTAT |
| SEQ ID NO: 44 | 44 | 56-FAM/ATA CAC CGA TGC AGA A/iMe-dC/T CAT GGG CTT ACG C |
| SEQ ID NO: 45 | 45 | 56-FAM/ATA CAC CGA TGC AGA ATT CAT GGG CTT ACG C |
| SEQ ID NO: 46 | 46 | 56-FAM/ATA CAC CGA TGC AGA ACT CAT GGG CTT ACG C |
| SEQ ID NO: 47 | 47 | 56-FAM/ATA CAC CGA TGC AGA A/ideoxyU/T CAT GGG CTT ACG C |
| SEQ ID NO: 48 | 48 | GGACACAGGGGTTGCCCAGGCTACAGATC5mCGGGTTCAGGCCCAGCCG |
| | | TGGGGACTGTTCACCTCTGAGAGGGCAGCCCTGCCCCGCGGCCCTGGCCC |
| | | TGCCCTGCACCCGGCTGGGTCTCATCTGACACGC |
| SEQ ID NO: 49 | 49 | GCGTGTCAGATGAGACCCAGCCGGGTGCAGGGCAGGGCCAGGGCCGCG |
| | | GGGCAGGGCTGCCCTCTCAGAGGTGAACAGTCCCCACGGCTGGGCCTGA |
| | | ACC5mCGGATCTGTAGCCTGGGCAACCCCTGTGTCC |
| SEQ ID NO: 50 | 50 | GGACACAGGGGTTGCCCAGGCTACAGATCCGGGTTCAGGCCCAGCCGTG |
| | | GGGACTGTTCACCTCTGAGAGGGCAGCCCTGCCCCGCGGCCCTGGCCCTG |
| | | CCCTGCACCCGGCTGGGTCTCATCTGACACGC |
| SEQ ID NO: 51 | 51 | GCGTGTCAGATGAGACCCAGCCGGGTGCAGGGCAGGGCCAGGGCCGCG |
| | | GGGCAGGGCTGCCCTCTCAGAGGTGAACAGTCCCCACGGCTGGGCCTGA |
| | | ACCCGGATCTGTAGCCTGGGCAACCCCTGTGTCC |
| SEQ ID NO: 52 | NP_ | MDSLLMNRRKFLYQFKNVRWAKGRRETYLCYVVKRRDSATSFSLDFGYLRNK |
| | 065712.1 | NGCHVELLFLRYISDWDLDPGRCYRVTWFTSWSPCYDCARHVADFLRGNPN |
| | homo | LSLRIFTARLYFCEDRKAEPEGLRRLHRAGVQIAIMTFKDYFYCWNTFVENHER |
| | sapiens | TFKAWEGLHENSVRLSRQLRRILLPLYEVDDLRDAFRTLGL |
| SEQ ID NO: 53 | G12 | NEKRKKTYDLAYMAMARIWAKQSYANKLKVGCLIVKDQLIISDGFNGVPSGL |
| | | PNRCEGPDGHTLDTVIHAEANAITKLARMGSSSEGATAYITAAPCMSCSKLLI |
| | | QAGIKRVVFNNPWKHSDGLELLKKVGVLVEQI |
| SEQ ID NO: 54 | 250 | NEQPIKRTQEEWDSFFLRFARDVAGMSKDPDRQVGAVVVTPDRRQLSLGYN |
| | | GFPPDVEDLPSLLADRDFKLANMVHAEVNCLRQAPFPLEGCTLYVTRFPCDR |
| | | CAERIVAAGVRRVVAPAPDFGHARWGSSWLQAAGSMESVGLAITLYKGE |
| SEQ ID NO: 55 | G10 | SLKERKWDTRWLKIAKEVSSWSKDPSKQIGAIAVRDNRMLAQGYNGLPRGL |
| | | PDTDQFLLGDIKYDIIIHAEENVIINAALNGVSLAGSTVYIVGLPPCSKCSGKLVN |
| | | AGVVRIIYVNEHDDKKWKDSFELSKIILEHAGVVCDKF |
| SEQ ID NO: 56 | | SXXXXXXXXXXXXXXXXXXXXSXXFXGXXXXXXXXXXXXXXXXXXXXXXXXXXX |
| | | X |
| SEQ ID NO: 57 | | SXXXXXXXXXXXXXXXXXXXXXXXXGXXXXXXXXXXXXXXXXXXXXXXXXXXX |
| | | X |
| SEQ ID NO: 58 | | SXXXXXXXXXXXXXXXXXXXXXXXXXXXXXXXXXSXXYXGXXXXXXXXXXXXXX |
| | | XXXXXXXXXXXXXXXXXXXXXXXXXXXXXXXXXXXXXXXXXXXXXXXXXXXXN |
| SEQ ID NO: 59 | | SXXXXXXXXXXXXXXXXXXXXXXXXXXXXXXXXXSXXFXGXXXXXXXXXXXXXX |
| | | XXXXXXXXXXXXXXXXXXXXXXXXXXXXXXXXXXXXXXXXXXXXXXXXXXXXN |

What is claimed is:

1. A sequencing method, comprising:
(a) contacting a DNA substrate comprising cytosine (C) and at least one methylcytosine nucleobase selected from 5-methylcytosine (5mC) and 5-hydroxymethylcytosine (5hmC) or comprising C and both 5mC and 5hmC, with a methylcytosine-selective deaminase to produce a deamination product,
wherein the methylcytosine-selective deaminase (i) is capable of deaminating 5mC to thymidine (T) and/or 5hmC to hydroxymethyluridine (hmU) and (ii) preferentially deaminates the at least one methylcytosine nucleobase relative to cytosine (C); and
(b) sequencing the deamination product, or amplifying the deamination product to produce an amplification product, and sequencing the amplification product, in each case, to produce sequence reads,
wherein positions of Cs and position of the at least one methylcytosine nucleobase in the DNA substrate is determined based on the sequence reads.

2. The method of claim 1, wherein the DNA substrate does not comprise DNA that has been pretreated to protect any nucleobase from deamination.

3. The method of claim 2, wherein the DNA substrate does not comprise DNA that has been pretreated to protect 5mC from deamination.

4. The method of claim 3, wherein the DNA substrate does not comprise DNA that has been pretreated with a TET methylcytosine dioxygenase.

5. The method of claim 1, wherein the DNA substrate does not comprise DNA that has been pretreated to protect C from deamination.

6. The method of claim 1, wherein the methylcytosine-selective deaminase preferentially deaminates 5mC relative to 5hmC and C, and the position of at least one 5mC in the DNA substrate is determined.

7. The method of claim 1, wherein the DNA substrate comprises DNA that has been pre-treated to protect one or more methylcytosine nucleobases from deamination.

8. The method of claim 7, wherein the pretreatment comprises incubation with an enzyme.

9. The method of claim 8, wherein the enzyme protects 5hmC from deamination.

10. The method of claim 9, wherein the positions of at least one 5mC, at least one 5hmC, and C are determined.

11. The method of claim 9, wherein the enzyme is selected from beta-glucosyltransferase (BGT), carbamoyl transferase, glycotransferase selective for 5hmC, and a combination thereof.

12. The method of claim 11, wherein the enzyme is BGT and the methylcytosine-selective deaminase does not deaminate glucosylated hmC.

13. The method of claim 8, wherein the enzyme protects C from unwanted background deamination by the methylcytosine-selective deaminase.

14. The method of claim 13, wherein the enzyme is selected from 4mC methylase and N4 methyltransferase.

15. The method of claim 8, wherein the enzyme protects 5mC from deamination.

16. The method of claim 15, wherein the enzyme is TET methylcytosine dioxygenase and the methylcytosine-selective deaminase does not deaminate 5-formylC (5fC) or 5-carboxyC (5caC).

17. The method of claim 8, wherein the pretreatment comprises incubation with a chemical reagent.

18. The method of claim 17, wherein the chemical reagent is pyridine borate.

19. The method of claim 1, wherein the methylcytosine-selective deaminase of (a) is a mixture of two or more methylcytosine-selective deaminases.

20. The method of claim 1, wherein the methylcytosine-selective deaminase of (a) does not deaminate C above background level.

21. The method of claim 1, wherein the methylcytosine-selective deaminase is selective for a methylcytosine nucleobase present within a defined sequence context, wherein the methylcytosine-selective deaminase does not deaminate the methylcytosine nucleobase lacking the defined sequence context above background level.

22. The method of claim 21, wherein the methylcytosine-selective deaminase comprises more than one methylcytosine-selective deaminase, each selective for 5mC and/or 5hmC present within different sequence contexts, wherein the methylcytosine selective deaminases do not deaminate the 5mC and/or 5hmC lacking the sequence context above background level.

23. The method of claim 1, wherein prior to (b) the DNA substrate is contacted with one or more deaminating agents having a different specificity from the methylcytosine-selective deaminase of (a).

24. The method of claim 23, wherein the deaminating agent is selected from an enzyme, a chemical reagent, and both.

25. The method of claim 1, wherein the deamination product is not amplified.

26. The method of claim 25, wherein the deamination product is added directly to a sequencing machine.

27. The method of claim 1, wherein the deamination product is amplified to produce an amplification product.

28. The method of claim 27, wherein amplification comprises polymerase chain reaction, optionally using Q5U polymerase, or a polymerase having a uracil-binding pocketQ5U polymerase, which is optionally Q5.

29. The method of claim 1, wherein before (b), the deamination product is contacted with a substance that generates a single nucleotide gap at the location of a uracil (U).

30. The method of claim 29, wherein the substance is selected from USER enzyme.

31. The method of claim 1, wherein before (b), the deamination product is contacted with a substance that generates an abasic site at the location of a U and/or an 5hmU.

32. The method of claim 31, wherein the substance is selected from uracil DNA glycosylase, single-strand-selective monofunctional uracil DNA glycosylase 1, thymine-DNA glycosylase and methyl-CpG binding domain protein 4.

33. The method of claim 1, wherein the DNA substrate further comprises an adaptor.

34. The method of claim 33, wherein the adaptor further comprises a sequencing primer binding site.

35. The method of claim 33, wherein the adaptor further comprises a barcode and/or unique molecular identifier (UMI).

36. The method of claim 1, wherein the DNA substrate comprises DNA selected from genomic DNA, ancient DNA, forensic DNA, environmental DNA, human DNA, free circulating DNA, synthetic DNA, plant DNA, and tumor DNA.

37. The method of claim 1, wherein the DNA substrate further comprises RNA.

38. The method of claim 1, wherein the methylcytosine-selective deaminase is a viral deaminase or non-natural modification thereof.

39. The method of claim 38, wherein the viral deaminase is a bacteriophage-encoded deaminase or modification thereof.

40. The method of claim 1, wherein the methylcytosine-selective deaminase comprises a B5 deaminase signature sequence.

41. The method of claim 1, wherein the methylcytosine-selective deaminase comprises an amino acid sequence that is at least 90% identical to any of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55.

42. The method of claim 1, wherein the methylcytosine-selective deaminase comprises an amino acid sequence selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54, and 55.

43. The method of claim 42, wherein the methylcytosine-selective deaminase comprises an amino acid selected from SEQ ID NOS:1, 5 and 14.

44. The method of claim 1, further comprising preparing the DNA substrate prior to (a) in a method comprising:
(a) ligating a hairpin adaptor to a double-stranded genomic DNA sample to produce a ligation product;
(b) enzymatically generating a free 3′ end in a double-stranded region of the hairpin adaptor in the ligation product;
(a) extending the free 3′ end in a reaction mix that comprises a strand-displacing or nick-translating polymerase, dGTP, dATP, dTTP and modified dCTP, wherein the modified dCTP is deamination resistant to produce and extended product;
to thereby produce a single-stranded DNA substrate.

45. A method for selective deamination of a DNA substrate, comprising:
contacting a DNA substrate comprising cytosine (C) and at least one methylcytosine nucleobase selected from 5-methylcytosine and 5-hydroxymethylcytosine, with a methylcytosine-selective deaminase, to produce a deamination product,
wherein the methylcytosine-selective deaminase (i) is capable of deaminating 5mC to thymidine (T) and/or 5hmC to hydroxymethyluridine (hmU), (ii) preferentially deaminates 5mC and/or 5hmC relative to cytosine (C), and (iii) and is characterized by having at least one feature selected from: (a) the methylcytosine-selective deaminase comprises a B5 deaminase signature sequence; and (b) the methylcytosine-selective deaminase is encoded by a viral gene located in a viral genome in proximity to a thymidylate synthase gene, and (c) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55.

46. A method for nucleobase editing, comprising:
contacting a fusion protein comprising a methylation-selective deaminase capable of deaminating 5mC to thymidine (T) fused to a DNA binding domain, with a target sequence comprising cytosine (C) and one or more 5-methylcytosine (5mC), to produce an edited target sequence comprising at least one T generated by deaminating 5mC,
wherein the methylation-selective deaminase preferentially deaminates 5mC relative to C, and is characterized by having at least one of the features selected from: (i) the methylcytosine-selective deaminase comprises B5 deaminase signature sequence and (ii) the methylcytosine-selective deaminase is encoded by a viral gene located in a viral genome in proximity to a thymidylate synthase gene, and (iii) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 53, 54 and 55.

47. The method of claim 46, wherein the methylcytosine-selective deaminase is selective for 5mC present within a sequence context, wherein the methylcytosine-selective deaminase does not deaminate 5mC lacking the sequence context above background level.

48. The method of claim 46, wherein the DNA binding domain is selected from a Cas9 domain, a Cas12 domain, a transcription activator-like effector nuclease (TALEN domain), a zinc finger (ZF) domain, a transcription activator-like effector (TALE) domain, and a methyl binding domain (MBD) domain.

49. A kit for methylation sequencing, comprising:
(a) a methylcytosine-selective deaminase that (i) is capable of deaminating 5-methyl cytosine (5mC) to thymidine (T) and/or 5-hydroxylmethyl cytosine (5hmC) to hydroxymethyluridine (hmU) and (ii) preferentially deaminates 5mC and/or 5hmC relative to cytosine (C); and
(b) one or more items selected from a primer; adaptor, polymerase; reverse transcriptase; ligase; protecting agent for selected nucleobase and/or modified nucleobase; another deaminase and/or deaminating treatment; reference DNA, methylated reference DNA; reference sequence; reaction buffer.

50. The kit of claim 49, wherein the methylcytosine-selective deaminase is characterized by having at least one of the features selected from (i) the methylcytosine-selective deaminase comprises a B5 deaminase signature sequence; and (ii) the methylcytosine-selective deaminase is encoded by a viral gene located in a viral genome in proximity to a thymidylate synthase gene, and (iii) the methylcytosine-selective deaminase comprises an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 53, 54 and 55.

51. A methylcytosine-selective deaminase, comprising:
a polypeptide that is capable of deaminating 5-methyl cytosine (5mC) to thymidine (T) and/or 5-hydroxylmethyl cytosine (5hmC) to hydroxymethyluridine (hmU); and
preferentially deaminates 5mC and/or 5hmC relative to cytosine (C);
wherein the polypeptide is selected from:
(i) a non-natural fusion protein comprising a methylcytosine-deaminase domain comprising a B5 deaminase signature sequence;
(ii) a non-natural fusion protein comprising a methylcytosine-selective deaminase domain encoded by a viral gene, wherein the viral gene is located in a viral genome in proximity to a thymidylate synthase gene;
(iii) a non-natural fusion protein comprising an amino acid sequence that is at least 90% identical to any of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 53 and 54; and
(iv) a polypeptide comprising an amino acid sequence selected from SEQ ID NOS: 37, 38, 39, and 40.

52. The deaminase of claim 51, wherein the polypeptide is a non-natural fusion protein comprising a methylcytosine-selective signature sequence, and the methylcytosine-selective deaminase portion of the fusion protein comprises is encoded by a viral gene.

53. A composition, comprising a methylcytosine deaminase of claim 51 and a component selected from a solid support, a buffer, and a reaction mixture.

54. A kit for deaminating a DNA substrate, comprising a deaminase of claim 51 and one or more items selected from a reaction buffer, primer; adaptor, polymerase; reverse transcriptase; ligase; protecting agent for selected nucleobase and/or modified nucleobase; another deaminase and/or deaminating treatment; reference DNA, methylated reference DNA; reference sequence; reaction buffer.


SEQUENCE LISTING (59 sequences)

SEQ ID NO: 1 - protein, 152 aa, synthetic construct
NEQPTKRTQEEWDSFFLRFARDVAGMSKDPDRQVGAVVVTPDRRQLSLGYNGFPTEVEDLPSLLADRDFKLANMVHAEVNCLRQAPFPLEGCTLYVTRFPCDRCAERIVASGVRRVVAPAPDFGHARWGSSWLQAAGSMESAGLAITLYKGE

SEQ ID NO: 2 - protein, 145 aa, synthetic construct
SQKQKLKQAFMSVAYDMASVSCCERGNHACVLVSRSLRIVSVGYNGPAARLHNGCARPLESGNCGCVHAEANAVLQPRIDHPFYAFVTAAPCEYCAQLLVNAGVRTVVYDVVTDSGHDGLKVLTNAGVGNFQFNQFSGDWLGLGS

SEQ ID NO: 3 - protein, 127 aa, synthetic construct
EVFKAFTQQLATLSKCTERQVAAIITDKNLTQVYSVGINGGPKGLADCMCVIDGKYGCIHAEINALIKDKSSDEGKVMFVTLAPCKQCAAAIINAPKGFSTVYYFEDWKEDSGIKLLTSAGITVAKI

SEQ ID NO: 4 - protein, 130 aa, synthetic construct
ACLVSTWSKDPQRGVGALLVSPDMRQVSWGFNGFPQGIADDPALLHDSQAKLERMVHAELNVILNSRRDVSGWTLYVTQHPCNHCALAIIQAGISMVVCPEPKPITSKWHVSHQIAAGLLEEAGVLVRRP

SEQ ID NO: 5 - protein, 152 aa, synthetic construct
HERPIKRTQEEWDSFFLRFARDVASMSKDPDRQVGAVVVTPDRRQLSLGFNGFPPGVEDLPSLLADRDFKLANMVHAEENCLRQAPFPLEGCALYVTRFPCHHCAEKIVAARVRRVVAPAPDFGHGRWGQSWLQSAESMESAGLAITLYKGE

SEQ ID NO: 6 - protein, 140 aa, synthetic construct
KRPSLEDTMMDIAGALALRSTCKKLCVGAVITDKRGRILGSGYNGVPTGMTHCIDVPCAGAQAPKGADLCEAVHAEANALLNCYDIDKATHIYVTHVPCLRCMKTLLNTPIKTIVYYHAANAELPALRIWASANRICTQI

SEQ ID NO: 7 - protein, 127 aa, synthetic construct; identical to SEQ ID NO: 3

SEQ ID NO: 8 - protein, 157 aa, synthetic construct
SSGLVPRGSHMALAVFISDWSKDPSSKVGAVISEGNRIVSVGYNGFPRGIVDDVNRLNDRQTRLKYTIHAELNAILQARETVRGYYLFTTHYPCSSCAQAIVQAGISRVYYRYSLGRWNESEAAQIFKEAEVLVKQVDIRRSDDLGGVSAYTRVGWE

SEQ ID NO: 9 - protein, 165 aa, synthetic construct
GSSHHHHHHTMNEQPTKRTQEEWDSFFLRFARDVAGMSKDPDRQVGAVVVTPDRRQLSLGYNGFPTEVEDLPSLLADRDFKLANMVHAEVNCLRQAPFPLEGCTLYVTRFPCDRCAERIVASGVRRVVAPAPDFGHARWGSSWLQAAGSMESAGLAITLYKGEIL

SEQ ID NO: 10 - protein, 152 aa, synthetic construct
SSGLVPRGSHMSKAEKFYSLACYHAQLFSKDPNTKVAALVIDNNNNIASVGYNGLPRGFEETSDRWEKPMKYNYVVHAQANAIATAARNGVRLDGCSIITTLFPCKECSKLIIQSGIRKVITSKPCKDSSWLESFSFSNEMFDECGIEVEYL

SEQ ID NO: 11 - protein, 157 aa, synthetic construct
SSGLVPRGSHMVSKWAKRFIQMAELVGSWSKDPSTQVGAVITKNNRIVSVGFNGYPHGISDSAEVDERETKYLKTLHAEENAILFAKRDLDGCEIWVTHFPCPNCAAKIIQTGISVVHCPQQTEDFLSRWGEKISLSQDMFEQAGVHVDWLPLEQLD

SEQ ID NO: 12 - protein, 155 aa, synthetic construct
ARPSIDQWAMYIARAVSTRASCLRRKVGCVLLNNDKRIDGTGYNGAASGLRHCDELKSCPSDFCQTGEGRDDCMAIHAEQNALLQCSDVSEINTCYVTRSPCTVCVKLLLNTGCKRIVYQELSSHDGAAILWLKAGRQWLSLDSAEMQAMEMLRR

SEQ ID NO: 13 - protein, 219 aa, synthetic construct
ESAAQTSHAACPTSLTLSTNVGNISNRDTGKKQMEKTNWDKRFMRLAIEARSWTKGLGAGIDRGVGACVVSPTHRQFSLGYAGFPRGIDDTADRLLSPEFRANHTVHAELNAILNASMSLEGWTIFSTECPCADCSNAIVQAGIKRVVAPSPNRNSNWYGSQLEGAETMREAGVTIERVTLAETGVVRVGASPQERAECGCNTPDVCASNGCQGKKRRR

SEQ ID NO: 14 - protein, 141 aa, synthetic construct
IDQSAWDARGMQLADDVASWSKDPDEGVGCVLASSDYRQFAFGVNGLPSGFPHDIVMDKQTKLKYSLHAEDNAIANAATDLRGWTAYVTKPPCLRCALALHRAGVKRVVFPELSPASSWYHAQKEAVQFMEANGLEVYLWA

SEQ ID NO: 15 - protein, 151 aa, synthetic construct
VIIMSHTKWEGRFYTLANTVADWSKDPHKKCGVVIISPDKKEISYGYNGFPTGIADDERLEQKETKNELMLHAEMNAILNAKKDLTGWTLYCSQSICTQCALAIIQANISRVVMPRIQLHSTWYLSCTNAVKLFTEANVIIHYINENEYYV

SEQ ID NO: 16 - protein, 159 aa, synthetic construct
ELMKKKIVSKTWSQRFMMMAKTISNNSKDPSTKVGSVAINPNNKQILALGYNGFPKGILDDDRLLDKNTKYSLMVHSEMNTIFNAALNGISLNGAYLYVYGLPTCSECAKGIIQVGIKKIFYVNTNPSKIWDEHWEKSKKMYDEVKIKYEELHEKDISI

SEQ ID NO: 17 - protein, 167 aa, synthetic construct
VVHKWRTRRTRGAPAHCRSPVEGVSDRRRGWTHMRVAHEYAMLSHSRKAKVGAVIVKDDRVVSVGYNGTVSGADNTCEYTADGQYLTYESVVHAEMNAIAFAASSGSPTNGCTMYLTLSPCFNCAKLIVQSGIVEVVYDRRWHKGGDSIAFLNDNGVLTRAQKDLPT

SEQ ID NO: 18 - protein, 148 aa, synthetic construct
QNLWDSRFMELAHLIGSWSKDDKRKVGCVIVSQDNRILSTGYNGFPRQLDGLSVIPAELKNVYTIHAEVNCMANLKAQLTEGVTVYVTYPPCLECSKMLKANIPIIRLVTSSLDAYSASKWHASMLEAKAFLTDSGVEYVCADSGYTS

SEQ ID NO: 19 - protein, 160 aa, synthetic construct
TKRPDFDDIYMELAVNLAKKSHCIKRHVGAVLTKDTRIISIGYNGPPEKTHNCDEEWPEEGCPRDSKGSCSLALHAEENAILFAVKNGTEVKGATIYVTLSPCISCARLLFASGVKKVIYLHSYAEYKGLAVDEGNAFLEKFGVTVEKFKGDFTLLNEEK

SEQ ID NO: 20 - protein, 159 aa, synthetic construct
SRPAFDDIFMELAVNLARRSHCIKRHVGAVLTKDTRIISIGYNGPPAGTHNCDEEWPQTGCPRDSKGGCSLAIHAEQNAILYAVKNHTSVEGSTLYVTLSPCLACARMIFTMGIKRVVYLHSYAEHKGLPDDEGITFLERFGVQTEKYKGQLAVVSPLI

SEQ ID NO: 21 - protein, 153 aa, synthetic construct
MNELNQNIRITLEELFMAIALIVKSRSICKVNQVGVVIAPKDLRNIYSFGYNGPVHKESHDVCNGISGNCGCLHAEINALLKVQVKDSEKIMFTTTSPCLYCAKAIIQSGFSVIYYFEPYRDLSPIQYLKKFNIEVFQLKTDFLTKFANTLKL

SEQ ID NO: 22 - protein, 529 aa, synthetic construct
DNKLLITSPEKSNKIDARTISNPELIFGIVGPIGVDIDNVVASLSKSLQGVGYKSTLIHLTKYINDSRISFDIDESSYFKRYKSLIQYGNEFRRIANSASALAGIGIAKIRERRSELTGVADQPALGTAYIIRQFKRAEEIDLMRSVYGRKFILISVFGSPIDRRRILIEKIRSYDSSPKSDAQCETEAIELIDIDHNQRDDDNGQRLSDVFHLGDVFVDGIDKDKAEGTIDRFIRAFFGDTSVSPTKDEYGLYIATAAALRSVDLSRQVGAAIFSKRGEVITLGCNEVPKANGGTYWSDDDNQAPQRDIELGRDPNQERKNEIIYDLLDRMFDEGLLSEKLSSVEDGQQRVDALLQNKKIKDAQLLDIIEFGRIIHAEMSAISDAARLGRETSGAILYCTTFPCHLCAKHIVAAGMDRVVFLEPYPKSYAQKLHADSITFDPTDESKVLFQPFMGISPRRYRDIFEKKKRKDAAGKAIRWYNKDPVPLLEDKSGAYIENEEGALIVALKDIFQASSSSKDASADAIEG

SEQ ID NO: 23 - protein, 132 aa, synthetic construct
RPSWDKIWIEFARNIAKRSYDPRFQVGSVIVTEDNCQVLAVGYNGNYAGGPNKVESEIPGESGFIHAEINALIKLDYNNPKKKKLYLTLAPCRMCAKAIVNAGIAEVIYEEPYRDMSGVQLLQDCGIKVRAF

SEQ ID NO: 24 - protein, 141 aa, synthetic construct
VRPNVKEYMINIAFATSERATCVRRKVGCVLTDINNHILSTGYNGVPKGVTHCIDRPCIAASSSSGTNLEGCKAIHAEVNAVAHCHDIQSVYAAYCTTLPCIHCIKLLLATNCQVIYYNQEYPHSLAKELWIDSGRELEQI

SEQ ID NO: 25 - protein, 148 aa, synthetic construct
KHDLRYMKLAKEVSTWSKDPSTKIGVVVIGTKGQVLSQGFNGFPRGIEDSDERLNNREVKYEYVVHGEMNAIFNATYNGVSLDGATMYVYNLPICSECAKGIIQVGIKRVVVLEADRGVRMRWIDSSKLSTDMFKESGVELTCLHTQL

SEQ ID NO: 26 - protein, 138 aa, synthetic construct
SSVGRPPWDEIWMGFAHSIARRSYDPRYQVGAVIVTKENTQVLAVGYNGNYSGGPNEVESIEPGNSGMIHAEINALLKCDYNIRGKKIMYVTLSPCKMCAKAMINGGIKEVVYDEEYRDLSGVELLMSAGVAVRRYTT

SEQ ID NO: 27 - protein, 153 aa, synthetic construct
TEGGFQKWDKRFMRLACEARTWVKGPDLGVGACVVSPDNRGFSLGYSGLPRGMKDTNDRITETEFKDHHMVHAELNAILNASRSVVGWTLYATTHPCSHCAGAIIQAGIKRVVAPMPPLHSDSRWAPSWAHARMAFKEAGVIFFPYEGDLPCD

SEQ ID NO: 28 - protein, 150 aa, synthetic construct
KTTQAEWDRRFLRLALEVRSWVKGSDLGVGAVIVDADRRPVSFGYSGLPRGMCDDTPLLTDPEFKDRHMVHAELNAILNASGPLRNCTIYVTKAPCAQCCSAIIQSGITRVVAPHPDDGRWKANQAAGRMALREAGVQWQGTKSLEVEGL

SEQ ID NO: 29 - protein, 155 aa, synthetic construct
PKTLKSKDDLVRSVMLLARTWAYSRSKDPKTKVGAAIYTIAGNVHLGYNGFPKNVPDWHDVWHELDPHINKHDLVIHAEANAIRKALKSGDSLGGAIMCVTLFPCIGCIKQCASVGLTQIIYADTKSKTPEAMLIADALGITLTPYTPGGLADHA

SEQ ID NO: 30 - protein, 153 aa, synthetic construct
VKKIMNNFEWLTDYRLKKISPDTLLLMMKQAKNARGSSYSPYSKFKVGCSILSEGGLVTCGTNFENQLYSKTVHAEVNAIGQMITMRERLLKCVFIASSNGVKPCVDCRKVISEFCDQDIPIVLINTTTDQVKIHTIYELAPTFFKLGQLKLL

SEQ ID NO: 31 - protein, 147 aa, synthetic construct
QSTRDSKERYFLKIAALVATRSTCPRRSVGCVIVNKHNHIKATGYNGVPRGYAHCIDTHCGGQNAASSKGLSSCMATHAEQNALLQCDDTMDIHTIYVTTSPCVTCSKLIANTSCKRVVYSEEYADRTGIEMLNKLAIGVYYERIHD

SEQ ID NO: 32 - protein, 312 aa, synthetic construct
VHPHCTGVQVMLVLIEGTDFMGKTHLAKQFQERFGLGYTHLSKPDEDFDYCSGYLAMMKDHHVFDRFHLGTLVYGSDLGCYKHTFDAIKQSKVNKSLKLRNALLILMYESNEDGFRTRLAGNDRAEMYNKDQIVAANESFLSQAITMRAFAELPNVFLYDVSQGWLEFDELLDRIKLETHWRPEIESVHRLEAQIQQSCQYRHVVAVAFDEYGQRIDHANNRTREGEICGKLSDTGPCGCVHAEDWLLSRLYVGRQSARIRTIALSCSPCSKCAQLIVDNGHVEFVVYGELYERDTRGVEILNEAGIKTLQA

SEQ ID NO: 33 - protein, 135 aa, synthetic construct
NKFDRKYLRMAKIWSENSHSKRLKVGALIVKDGSIISDGYNGTPSGFDNTCENDNNETLPEVLHAEANAITKIAKSHNSSEGATIYVLVSPCLSCSKLIIQAGIKRVVYNTKYRLQDGIKLLEKAGVVIDEIEDL

SEQ ID NO: 34 - protein, 135 aa, synthetic construct
CRRAEKLLTFARFAKELSLLATCKRNGVGCVICDPLFTHVYSLGYNGWPRGFDHDRCVKTNDPCGCVHAEINACLKLDTSRSDLICIVTEVPCIRCSQAIIQTHRIQRVVVYQPTYPTTRGPDLLKEAGLQYDQM

SEQ ID NO: 35 - protein, 134 aa, synthetic construct
EPSKKHDLYMRIADAVALSSHCKRSKVGCIIVKNDNIIGIGYNGTPSGFENDCECNNTTKEEVLHAESNAITKCAKSTISSDGSIMYCTLMPCFNCAKLIIQSGIKQVIYKDDYRNKSGIKLLTKANIEVNRYE

SEQ ID NO: 36 - protein, 223 aa, synthetic construct
YRMYTQLQALRRLSKCADKQVACIITDVEDNILSVGVNRVFNCVDCTKTGSKVGCLVSHAEEAALANLARTYEAKRAYLTLYPCENCQRVLDPVVDEIIVFGKQHKNCVIDPNKIILVPDLSKELWGTNGEQKQLSVITSELAELIKVITDFFYRRNERATKIEEVLDEIVDVELMTAVLLDLISARYDESVLFELAKIKEAKLGKIKRRLADGTIKRDSPYS

SEQ ID NO: 37 - protein, 154 aa, synthetic construct
GSNERPIKRTQEEWDRFFLRFARDVAGMSKDPDRQVGAVVVTPDRRQLSLGYNGFPPGVEDLPSLLADRDFKLANMVHAEENCLRQAPFPLEGCTLYVTRFPCHHCAEKIVAAGVRRVVAPAPDFGHARWGQSWLQSAGLMESAGIAITLYKGE

SEQ ID NO: 38 - protein, 148 aa, synthetic construct
TQDKWDRRFMRLAKEVATWSKDPDRGVGAVIVSPDRRQVSWGYNGFPRGIADDPERLNDKELKNQLMVHAELNAILNASRDVTGWTLYVTKHPCSHCASAIIQAGITRVVCPEPDPADSRWHESHQLARKLLEEAGVEVTHFEPDECA

SEQ ID NO: 39 - protein, 143 aa, synthetic construct
DQSKWDARFMQLADDVASWSKDPDKGVGAVLVSPDKRQFSFGYNGLPSGFPDDVVMDKETKNRYTLHAEENAIANAAQDLRGWTLYVTKAPCLRCALAIHRAGITRVVFPELDPSSSWYASQREAVEFLEAAGVEVARFKPCA

SEQ ID NO: 40 - protein, 146 aa, synthetic construct
TMDKWDRRFMRLALEVRSWVKGPDRGVGAVIVSPDRRQVSFGYSGLPRGMADDEERLTDPEFKDRHMVHAELNAILNASRSVRGWTLYCTKAPCAHCCSAIIQAGITRVVSPSPDDSRWHRSQLEGREALREAGVEVERMEQEEAA

SEQ ID NO: 41 - DNA, 20 bp, synthetic construct
  source 1..20: mol_type = other DNA
  variation 14: note = 5mC
tgtccgatagactctacgca

SEQ ID NO: 42 - DNA, 20 bp, synthetic construct
  source 1..20: mol_type = other DNA
  variation 14: note = 5hmC
tgtccgatagactctacgca

SEQ ID NO: 43 - DNA, 31 bp, synthetic construct
  source 1..31: mol_type = other DNA
gcgtaagcccatgaattctgcatcggtgtat

SEQ ID NO: 44 - DNA, 31 bp, synthetic construct
  source 1..31: mol_type = other DNA
  variation 1: note = 56-FAM
  variation 17: note = iMe-dC
atacaccgatgcagaactcatgggcttacgc

SEQ ID NO: 45 - DNA, 31 bp, synthetic construct
  source 1..31: mol_type = other DNA
  variation 1: note = 56-FAM
atacaccgatgcagaattcatgggcttacgc

SEQ ID NO: 46 - DNA, 31 bp, synthetic construct; identical to SEQ ID NO: 44
  source 1..31: mol_type = other DNA
  variation 1: note = 56-FAM

SEQ ID NO: 47 - DNA, 31 bp, synthetic construct
  source 1..31: mol_type = other DNA
  variation 17: note = n=ideoxyU
atacaccgatgcagaantcatgggcttacgc

SEQ ID NO: 48 - DNA, 131 bp, synthetic construct
  source 1..131: mol_type = other DNA
  variation 30: note = 5mC
ggacacaggggttgcccaggctacagatccgggttcaggcccagccgtggggactgttcacctctgagagggcagccctgccccgcggccctggccctgccctgcacccggctgggtctcatctgacacgc

SEQ ID NO: 49 - DNA, 131 bp, synthetic construct
  source 1..131: mol_type = other DNA
  variation 101: note = 5mC
gcgtgtcagatgagacccagccgggtgcagggcagggccagggccgcggggcagggctgccctctcagaggtgaacagtccccacggctgggcctgaacccggatctgtagcctgggcaacccctgtgtcc

SEQ ID NO: 50 - DNA, 131 bp, synthetic construct; identical to SEQ ID NO: 48
  source 1..131: mol_type = other DNA

SEQ ID NO: 51 - DNA, 131 bp, synthetic construct; identical to SEQ ID NO: 49
  source 1..131: mol_type = other DNA

SEQ ID NO: 52 - protein, 198 aa, Homo sapiens [sequence omitted]

SEQ ID NO: 53 - protein, 137 aa, synthetic construct
NEKRKKTYDLAYMAMARIWAKQSYANKLKVGCLIVKDQLIISDGFNGVPSGLPNRCEGPDGHTLDTVIHAEANAITKLARMGSSSEGATAYITAAPCMSCSKLLIQAGIKRVVFNNPWKHSDGLELLKKVGVLVEQI

SEQ ID NO: 54 - protein, 152 aa, synthetic construct
NEQPIKRTQEEWDSFFLRFARDVAGMSKDPDRQVGAVVVTPDRRQLSLGYNGFPPDVEDLPSLLADRDFKLANMVHAEVNCLRQAPFPLEGCTLYVTRFPCDRCAERIVAAGVRRVVAPAPDFGHARWGSSWLQAAGSMESVGLAITLYKGE

SEQ ID NO: 55 - protein, 145 aa, synthetic construct
SLKERKWDTRWLKIAKEVSSWSKDPSKQIGAIAVRDNRMLAQGYNGLPRGLPDTDQFLLGDIKYDIIIHAEENVIINAALNGVSLAGSTVYIVGLPPCSKCSGKLVNAGVVRIIYVNEHDDKKWKDSFELSKIILEHAGVVCDKF

SEQ ID NO: 56 - protein, 55 aa, synthetic construct
SXXXXXXXXXXXXXXXXXXXXSXXFXGXXXXXXXXXXXXXXXXXXXXXXXXXXXX

SEQ ID NO: 57 - protein, 55 aa, synthetic construct
SXXXXXXXXXXXXXXXXXXXXSXXYXGXXXXXXXXXXXXXXXXXXXXXXXXXXXX

SEQ ID NO: 58 - protein, 107 aa, synthetic construct
SXXXXXXXXXXXXXXXXXXXXXXXXXXXXXXXXXSXXYXGXXXXXXXXXXXXXXXXXXXXXXXXXXXXXXXXXXXXXXXXXXXXXXXXXXXXXXXXXXXXXXXXXXN

SEQ ID NO: 59 - protein, 107 aa, synthetic construct
SXXXXXXXXXXXXXXXXXXXXXXXXXXXXXXXXXSXXFXGXXXXXXXXXXXXXXXXXXXXXXXXXXXXXXXXXXXXXXXXXXXXXXXXXXXXXXXXXXXXXXXXXXN